(19) United States
(12) Patent Application Publication
(10) Pub. No.: US 2026/0263570 A1
(43) Pub. Date: Sep. 10, 2026

(54) ACTIVATORS OF COAGULATION FACTOR X AND FORMULATIONS THEREOF FOR TREATING BLEEDING DISORDERS

(71) Applicant: JIANGSU BIOJETAY BIOTECHNOLOGY CO., LTD., Wuxi (CN)

(72) Inventors: Zan ZHOU, Beijing (CN); Xiaomin WANG, Beijing (CN); Bingzhang WANG, Beijing (CN); Junyong LIU, Beijing (CN)

(73) Assignee: JIANGSU BIOJETAY BIOTECHNOLOGY CO., LTD., Wuxi (CN)

(21) Appl. No.: 18/875,540

(22) PCT Filed: Jun. 19, 2023
(86) PCT No.: PCT/CN2023/100987

§ 371 Date: Dec. 16, 2024

(30) Foreign Application Priority Data
Jun. 20, 2022 (WO) PCT/CN2022/099820

(51) Int. Cl. A61K 38/48; A61K 9/00; A61K 47/02; A61K 47/18; A61K 47/20; A61K 47/26; A61K 47/34; A61P 7/04

(52) U.S. Cl. CPC A61K 38/4886; A61K 9/0019; A61K 47/02; A61K 47/183; A61K 47/20; A61K 47/26; A61K 47/34; A61P 7/04; C12Y 304/24058

(57) ABSTRACT

Provided herein are compositions (e.g., pharmaceutical compositions) of coagulation factor X activator (FX activator, e.g., RVV-X) comprising sucrose, histidine, polysorbate 20, and mannitol, and uses of FX activators (e.g., RVV-X) or compositions (e.g., pharmaceutical compositions) thereof for treating a bleeding disorder (e.g., hemophilia) in an individual (e.g., human).

### SUBMISSION OF SEQUENCE LISTING ON ASCII TEXT FILE

The content of the following submission on ASCII text file is incorporated herein by reference in its entirety; a computer readable form (CRF) of the Sequence Listing (file name: EN_202206200179_SEQLIST, date recorded: Jun. 20, 2022, size: 9 KB).

### FIELD OF THE INVENTION

The present invention relates to compositions (e.g., pharmaceutical compositions) of coagulation factor X activator (FX activator), and uses of FX activators or compositions (e.g., pharmaceutical compositions) thereof for treating a bleeding disorder (e.g., hemophilia) in an individual (e.g., human).

### BACKGROUND OF THE INVENTION

Hemostasis is a complex physiological process that leads to the cessation of bleeding. Under normal situations, immediately follow tissue injury, platelets, plasma proteins, and blood vessels and endothelial cells collaboratively participate in hemostasis, resulting in the rapid formation of a clot. During the coagulation cascade, certain plasma proteins (or coagulation factors) are sequentially activated in a “cascade” by another previously activated coagulation factor, leading to the rapid generation of thrombin.

Coagulation factor X (FX) is a vitamin K-dependent serine protease, its active form (FXa) is the only physiological activator of prothrombin in vivo, and plays a key role in the common coagulation pathway. Coagulation factor X activator (FX activator) can specifically activate FX, fully expose its active site to generate FXa. FXa then forms a prothrombin complex with activated platelets, FVa, and calcium ions at the injury site, thereby increasing thrombin generation. Thrombin activates platelets and factors V and VIII at the injury site, and forms thrombus through the conversion of fibrinogen to fibrin, in order to achieve the purpose of hemostasis in bleeding patients. FX activator has potentially great application value in surgical wound bleeding, medical bleeding diseases, coagulation system diseases, such as hemophilia.

The earliest discovered and so far the most active FX activator is extracted from the venom of the eastern Russell's viper (Daboia russellii siamensis), abbreviated as RVV-X. RVV-X is a metalloprotease with a relative molecular weight (MW) of 92,880 Da, which is composed of a heavy chain with an MW of about 57,600 Da and two light chains with a MW of about 16,400 Da and about 19,400 Da through disulfide bonds.

Since McFarlane first discovered the factor X activator RVV-X in the venom of the eastern Russell's viper (R. G. MacFarlane, “The coagulant action of Russell's viper venom; the use of antivenom in defining its reaction with a serum factor,” Br J Haenatol. 1961; 7:496-511), FX activators have also been found in many other snake venoms. FX activators are commonly found in viper and rattlesnake venom. Agents that can activate FX have also been found in a few cobra venoms. In addition, FX activators can also be obtained by bioengineering, by expressing recombinant human FX activator in cells, and isolating and purifying the recombinant human FX activator from the cell culture media.

Biological activity of FX activators depends on the secondary and tertiary structures thereof, and thus it is particularly important to maintain its biological activity during preparation, purification, storage and administration.

Thrombin and FX activator are usually prepared together into a composition for use as a hemostatic drug, for example, see CN1520880A, CN1727002A, CN108785665A, and CN1810258A. As a macromolecular protein, if FX activator is used as the single active ingredient to prepare for FX activator pharmaceutical composition, FX activators may be prone to denaturation and not suitable for long-term storage. Human serum albumin (HSA) is a commonly used protective agent, which has been widely used in biological products as a protein stabilizer. However, HSA has been gradually eliminated due to its tendency to cause allergic reactions and the potential risk of virus contamination.

Bleeding disorders include congenital bleeding disorders and acquired bleeding disorders, such as a bleeding disorder due to a deficiency of a coagulation factor (e.g., FX), or due to the presence of acquired inhibitors (e.g., autoantibodies) to a coagulation factor, etc.

Haemophilia is a mostly inherited genetic disorder that impairs the body's ability to make blood clots to stop bleeding. Haemophilia A (HA) and haemophilia B (HB) are the two main types of haemophilia. Haemophilia A occurs due to low amounts of clotting factor VIII (FVIII), and haemophilia B occurs due to low levels of clotting factor IX (FIX). Haemophilia C (HC) is another type of haemophilia that occurs due to low levels of factor XI (FXI). Parahaemophilia occurs due to low levels of factor V (FV). Acquired haemophilia can be associated with cancers, autoimmune disorders, or pregnancy.

Bleeding episodes (BEs) in HA and HB patients are usually treated with factor replacement therapy; however, 20%/—30% of patients with HA, and 5% with HB, will develop inhibitors to exogenous FVIII or FIX. HA and HB with inhibitors (e.g., autoantibodies) are difficult to control. In these patients, hemostasis may not be achievable with replacement of the deficient factor (depending upon the inhibitor titer) and thus may require administration of bypassing agents. Although bypass treatment agents, such as activated thrombin complex concentrates (aPCC, e.g., Feiba®), emicizumab-kxwh (e.g., HEMLIBRA®), and activated recombinant human coagulation Factor VII (rFVIIa, e.g., NovoSeven® RT), have been widely used as hemostatic treatment agents, there are still shortcomings and limitations. For example, aPCC is not available in China, trace amounts of FVIII in aPCC may induce an anamnestic response to FVIII; and rFVIIa and emicizumab are not widely used because of high price.

The disclosures of all publications, patents, patent applications and published patent applications referred to herein are hereby incorporated herein by reference in their entirety.

### BRIEF SUMMARY OF THE INVENTION

One aspect of the present application provides a pharmaceutical composition comprising i) a coagulation factor X activator (FX activator; e.g., RVV-X) in an amount of from about 0.1 U/mL to about 200 U/mL; ii) a stabilizer in an amount of from about 2 mg/ml to about 100 mg/ml; iii) a buffering agent in an amount of from about 0.1 mg/ml to about 50 mg/ml; iv) a surfactant in an amount of from about 0.001% (w/v) to about 0.1% (w/v); and v) a tonicity agent in an amount of from about 1 mg/ml to about 100 mg/ml; wherein the pharmaceutical composition has a pH of from about 6.0 to about 8.0.

In some embodiments according to any one of the pharmaceutical compositions described above, the FX activator is RVV-X. In some embodiments, the RVV-X comprises: a) i) a heavy chain comprising the sequence of SEQ ID NO: 1, or a sequence with at least about 80% identity to SEQ ID NO: 1; ii) a light chain 1 comprising the sequence of SEQ ID NO: 2, or a sequence with at least about 80% identity to SEQ ID NO: 2; and iii) a light chain 2 comprising the sequence of SEQ ID NO: 3, or a sequence with at least about 80% identity to SEQ ID NO: 3;) i) a heavy chain comprising the sequence of SEQ ID NO: 1, or a sequence with at least about 80% identity to SEQ ID NO: 1; ii) a light chain 1 comprising the sequence of SEQ ID NO: 5, or a sequence with at least about 80% identity to SEQ ID NO: 5; and iii) a light chain 2 comprising the sequence of SEQ ID NO: 3, or a sequence with at least about 80% identity to SEQ ID NO: 3; or c) a mixture of a) and b). In some embodiments, the RVV-X is isolated from Daboia russellii siamensis venom. In some embodiments, the purity of the RVV-X in the pharmaceutical composition is at least about 95% (e.g., at least about any of 96%, 97%, 98%, 99%, or 100%).

In some embodiments according to any one of the pharmaceutical compositions described above, the FX activator (e.g., RVV-X) is in an amount of from about 1 U/mL to about 100 U/mL, such as from about 5 U/mL to about 100 U/mL, from about 5 U/mL to about 50 U/mL, or about 10 U/mL.

In some embodiments according to any one of the pharmaceutical compositions described above, the stabilizer comprises one or both of sucrose and trehalose. In some embodiments, the stabilizer is sucrose. In some embodiments, the stabilizer (e.g., sucrose) is in an amount of from about 2 mg/ml to about 60 mg/ml, such as from about 15 mg/ml to about 60 mg/ml, from about 30 mg/ml to about 50 mg/ml, or about 30 mg/ml.

In some embodiments according to any one of the pharmaceutical compositions described above, the buffering agent comprises one or both of histidine and arginine. In some embodiments, the buffering agent is histidine. In some embodiments, the buffering agent (e.g., histidine) is in an amount of from about 2 mg/ml to about 20 mg/ml, such as from about 2 mg/ml to about 15 mg/ml, from about 3 mg/ml to about 5 mg/ml, or about 3 mg/ml.

In some embodiments according to any one of the pharmaceutical compositions described above, the surfactant comprises one or both of polysorbate and poloxamer. In some embodiments, the surfactant is selected from one or more of polysorbate 20, polysorbate 80, and poloxamer 188. In some embodiments, the surfactant is polysorbate 20. In some embodiments, the surfactant (e.g., polysorbate 20) is in an amount of from about 0.005% (w/v) to about 0.05% (w/v), such as from about 0.01% (w/v) to about 0.05% (w/v), from about 0.01% (w/v) to about 0.03% (w/v), or about 0.02% (w/v).

In some embodiments according to any one of the pharmaceutical compositions described above, the tonicity agent is mannitol. In some embodiments, the tonicity agent is in an amount of from about 10 mg/ml to about 60 mg/ml, such as from about 30 mg/ml to about 60 mg/ml, or about 40 mg/ml.

In some embodiments according to any one of the pharmaceutical compositions described above, the pharmaceutical composition has a pH of from about 6.3 to about 7.3, such as from about 6.8 to about 7.0, or about 6.85.

In some embodiments according to any one of the pharmaceutical compositions described above, the pharmaceutical composition further comprises an antioxidant, such as methionine. In some embodiments, the antioxidant is in an amount of from about 0.01 mg/ml to about 1 mg/ml, such as from about 0.05 mg/ml to about 1 mg/ml.

In some embodiments according to any one of the pharmaceutical compositions described above, the pharmaceutical composition further comprises a calcium salt, such as calcium chloride. In some embodiments, the calcium salt is in an amount of from about 0.1 mg/ml to about 10 mg/ml.

In some embodiments according to any one of the pharmaceutical compositions described above, the pharmaceutical composition comprises: i) RVV-X in an amount of about 10 U/mL; ii) sucrose in an amount of about 30 mg/ml; iii) histidine in an amount of about 3 mg/ml; iv) polysorbate 20 in an amount of about 0.02% (w/v); and v) mannitol in an amount of about 40 mg/ml; wherein the pharmaceutical composition has a pH of about 6.85.

In some embodiments according to any one of the pharmaceutical compositions described above, the pharmaceutical composition is lyophilized. In some embodiments, the pharmaceutical composition is sterile. In some embodiments, the pharmaceutical composition is stable i) at 25° C. for at least about 4 hours after reconstitution; ii) at 2-8° C. for at least about 3 months, such as after reconstitution; and/or iii) at 25° C. under accelerated stability condition for at least about 3 months after reconstitution. In some embodiments, the pharmaceutical composition after reconstitution comprises less than about 5% of insoluble particles with a diameter of more than 10 μm after storage at 25° C. under accelerated stability condition for about 6 months.

Another aspect of the present application provides a method of treating a bleeding disorder (e.g., hemophilia, such as hemophilia A or hemophilia B) in an individual (e.g., human), comprising administering to the individual an effective amount of any one of the pharmaceutical compositions described above.

Another aspect of the present application provides a method of treating a bleeding disorder (e.g., hemophilia, such as hemophilia A or hemophilia B) in an individual (e.g., human), comprising administering to the individual an effective amount of an FX activator (e.g., RVV-X), wherein the FX activator is administered in a dose of from about 0.01 U/kg to about 0.48 U/kg. In some embodiments, the FX activator is administered in a dose of from about 0.08 U/kg to about 0.48 U/kg, such as from about 0.01 U/kg to about 0.16 U/kg, from about 0.08 U/kg to about 0.16 U/kg, from about 0.1 U/kg to about 0.48 U/kg, or from about 0.1 U/kg to about 0.16 U/kg. In some preferred embodiments, the FX activator is administered in a dose of about 0.08 U/kg, 0.1 U/kg, 0.12 U/kg, 0.14 U/kg or 0.16 U/kg. In some embodiments, the FX activator is administered once. In some embodiments, the FX activator is administered for a maximum of 6 doses. In some embodiments, the FX activator is administered for 4 doses. In some embodiments, the FX activator is administered every 4 hours (q4h) to every 8 hours (q8h), such as q4h. In some embodiments, the FX activator is administered once in a dose of from about 0.01 U/kg to about 0.48 U/kg. In some embodiments, the FX activator is administered in a dose of about 0.16 U/kg q4 h for a maximum of 6 doses. In some embodiments, the FX activator is administered in a dose of about 0.16 U/kg q4 h for 4 doses. In some preferred embodiments, the FX activator is administered in a dose of about 0.1 U/kg q4 h for a maximum of 6 doses. In some preferred embodiments, the FX activator is administered in a dose of about 0.1 U/kg q4 h for 4 doses.

In some embodiments according to any one of the methods described above, the FX activator is RVV-X. In some embodiments, the RVV-X comprises: a) i) a heavy chain comprising the sequence of SEQ ID NO: 1, or a sequence with at least about 80% identity to SEQ ID NO: 1; ii) a light chain 1 comprising the sequence of SEQ ID NO: 2, or a sequence with at least about 80% identity to SEQ ID NO: 2; and iii) a light chain 2 comprising the sequence of SEQ ID NO: 3, or a sequence with at least about 80% identity to SEQ ID NO: 3; b) i) a heavy chain comprising the sequence of SEQ ID NO: 1, or a sequence with at least about 80% identity to SEQ ID NO: 1; ii) a light chain 1 comprising the sequence of SEQ ID NO: 5, or a sequence with at least about 80% identity to SEQ ID NO: 5; and iii) a light chain 2 comprising the sequence of SEQ ID NO: 3, or a sequence with at least about 80% identity to SEQ ID NO: 3; or c) a mixture of a) and b). In some embodiments, the RVV-X is isolated from Daboia russellii siamensis venom. In some embodiments, the purity of the RVV-X is at least about 95% (e.g., at least about any of 96%, 97%, 98%, 99%, or 100%).

In some embodiments according to any one of the methods described above, wherein the FX activator is contained in a pharmaceutical composition. In some embodiments, the concentration of the FX activator in the pharmaceutical composition is from about 0.1 U/mL to about 200 U/mL, such as from about 1 U/mL to about 100 U/mL, from about 5 U/mL to about 100 U/mL, from about 5 U/mL to about 50 U/mL, or about 10 U/mL. In some embodiments, the pharmaceutical composition further comprises one or more of a stabilizer, a buffering agent, a surfactant, and a tonicity agent. In some embodiments, the stabilizer comprises one or both of sucrose and trehalose. In some embodiments, the stabilizer is sucrose. In some embodiments, the stabilizer (e.g., sucrose) is in an amount of from about 2 mg/ml to about 100 mg/ml, such as from about 2 mg/ml to about 60 mg/ml, from about 15 mg/ml to about 60 mg/ml, from about 30 mg/ml to about 50 mg/ml, or about 30 mg/ml. In some embodiments, the buffering agent comprises one or both of histidine and arginine. In some embodiments, the buffering agent is histidine. In some embodiments, the buffering agent (e.g., histidine) is in an amount of from about 0.1 mg/ml to about 50 mg/ml, such as from about 2 mg/ml to about 20 mg/ml, from about 2 mg/ml to about 15 mg/ml, from about 3 mg/ml to about 5 mg/ml, or about 3 mg/ml. In some embodiments, the surfactant comprises one or both of polysorbate and poloxamer. In some embodiments, the surfactant is selected from one or more of polysorbate 20, polysorbate 80, and poloxamer 188. In some embodiments, the surfactant is polysorbate 20. In some embodiments, the surfactant (e.g., polysorbate 20) is in an amount of from about 0.001% (w/v) to about 0.1% (w/v), such as from about 0.005% (w/v) to about 0.05% (w/v), from about 0.01% (w/v) to about 0.05% (w/v), from about 0.01% (w/v) to about 0.03% (w/v), or about 0.02% (w/v). In some embodiments, the tonicity agent is mannitol. In some embodiments, the tonicity agent is in an amount of from about 1 mg/ml to about 100 mg/ml, such as from about 10 mg/ml to about 60 mg/ml, from about 30 mg/ml to about 60 mg/ml, or about 40 mg/ml. In some embodiments, the pharmaceutical composition has a pH of from about 6.0 to about 8.0, such as from about 6.3 to about 7.3, from about 6.8 to about 7.0, or about 6.85. In some embodiments, the pharmaceutical composition further comprises an antioxidant, the antioxidant is methionine. In some embodiments, the antioxidant is in an amount of from about 0.01 mg/ml to about 1 mg/ml, such as from about 0.05 mg/ml to about 1 mg/ml. In some embodiments, the pharmaceutical composition further comprises a calcium salt. In some embodiments, the calcium salt is calcium chloride. In some embodiments, the calcium salt is in an amount of from about 0.1 mg/ml to about 10 mg/ml. In some embodiments, the pharmaceutical composition comprises: i) RVV-X in an amount of about 10 U/mL; ii) sucrose in an amount of about 30 mg/ml; iii) histidine in an amount of about 3 mg/ml; iv) polysorbate 20 in an amount of about 0.02% (w/v); and v) mannitol in an amount of about 40 mg/ml; wherein the pharmaceutical composition has a pH of about 6.85. In some embodiments, the pharmaceutical composition is lyophilized. In some embodiments, the pharmaceutical composition is sterile. In some embodiments, the pharmaceutical composition is stable i) at 25° C. for at least about 4 hours after reconstitution; ii) at 2-8° C. for at least about 3 months, such as after reconstitution; and/or iii) at 25° C. under accelerated stability condition for at least about 3 months after reconstitution. In some embodiments, the pharmaceutical composition after reconstitution comprises less than about 5% of insoluble particles with a diameter of more than 10 μm after storage at 25° C. under accelerated stability condition for about 6 months.

In some embodiments according to any one of the methods described above, the FX activator (e.g., RVV-X) is administered intravenously.

In some embodiments according to any one of the methods described above, the bleeding disorder is a congenital bleeding disorder or an acquired bleeding disorder. In some embodiments, the bleeding disorder is selected from the group consisting of a disorder due to a deficiency of a coagulation factor, a disorder due to the presence of acquired inhibitors to a coagulation factor, a hematologic disorder, a hemorrhagic disorder, Von Willebrands' disease, a disorder resulting from anticoagulant therapy with a vitamin-K antagonist, hereditary platelet disorders, vitamin K epoxide reductase Cl deficiency, gamma-carboxylase deficiency, bleeding associated with trauma, injury, or surgery, thrombosis, thrombocytopenia, stoke, coagulopathy, disseminated intravascular coagulation (DIC), Bernard Soulier syndrome, Glanzman thromblastemia, and storage pool deficiency. In some embodiments, the bleeding disorder is due to a deficiency of a coagulation factor. In some embodiments, the bleeding disorder is hemophilia, such as hemophilia A or hemophilia B. In some embodiments, the hemophilia is hemophilia with inhibitors.

In some embodiments according to any one of the methods described above, the individual is a human.

Also provided are compositions (e.g., pharmaceutical compositions), kits, and articles of manufacture comprising any of pharmaceutical compositions described herein. Methods of treating a bleeding disorder (e.g., hemophilia, such as hemophilia A or hemophilia B) in an individual (e.g., human) using any of the methods described herein or pharmaceutical compositions thereof are also provided.

### BRIEF DESCRIPTION OF THE DRAWINGS

FIGS. 1A-ID depict PK characteristics of single injection and multiple injections of RVV-X. Patients received a single intravenous administration of RVV-X in three dose cohorts of 0.16 U/kg, 0.32 U/kg and 0.48 U/kg. FIG. 1A shows the plasma RVV-X concentrations after single injection in concentration-time curve. FIG. 1B shows the plasma RVV-X concentrations after single injection in semi-logarithmic curve of drug concentration-time. FIG. 1C shows the analysis of plasma RVV-X concentrations after single injection between C. and dose. FIG. 1D shows the plasma RVV-X concentrations after multiple injections in concentration-time curve.

FIGS. 2A-2B depict pharmacodynamics (PD) characteristics of of RVV-X in single-dose part. Patients received a single intravenous administration of of RVV-X at six doses of 0.01 U/kg, 0.04 U/kg, 0.08 U/kg, 0.16 U/kg, 0.32 U/kg, and 0.48 U/kg. FIG. 2A shows the change of peak height for thrombin generation (TG). FIG. 2B shows the change of endogenous thrombin-generating potential (ETP). The change value was obtained by subtracting the baseline value from the measured value of each tested time point after RVV-X administration.

FIGS. 3A-3B depict FX plasma concentrations (FX:C) after single intravenous administration of RVV-X. Patients received a single intravenous administration of RVV-X at six doses of 0.01 U/kg, 0.04 U/kg, 0.08 U/kg, 0.16 U/kg, 0.32 U/kg, and 0.48 U/kg. FIG. 3A shows the change of FX:C. FIG. 3B shows the value of FX:C. Results are presented as mean±standard deviation.

FIG. 4 depicts relationship between RVV-X concentration and APTT in plasma samples.

FIG. 5 shows the number of insoluble particles in each lyophilized formulation in 0 month.

FIG. 6 shows the number of insoluble particles in each lyophilized formulation after 6-month of acceleration.

### DETAILED DESCRIPTION OF THE INVENTION

Thrombin and FX activator are usually prepared together into a composition for use as a hemostatic drug. As a macromolecular protein, if FX activator is used as the single active ingredient to prepare for FX activator pharmaceutical composition, FX activators may be prone to denaturation and not suitable for long-term storage. Further, the biological activity of FX activators depends on the secondary and tertiary structures thereof, and thus it is particularly important to maintain its biological activity during preparation, purification, storage and administration.

Bleeding episodes of hemophilia A (HA) or B (HB) patients with inhibitor (e.g., autoantibody) are difficult to control. Current bypass treatment agents have shortcomings and limitations. For example, activated thrombin complex concentrates (aPCC, e.g., Feiba®) are not widely available. Further, trace amounts of FVIII in aPCC may induce an anamnestic response to FVIII. PCC may induce immune memory response to FIX. Moreover, PCC and aPCC are derived from blood products, thus carry potential risks of viral infection and allergy. Emicizumab-kxwh (Hemlibra®) is a bispecific humanized monoclonal antibody that restores the function of missing activated FVIII by bridging activated FIX and FX to facilitate effective hemostasis. It has been approved by FDA for treating HA and HB patients with FVIII or FIX inhibitors, respectively. However, it is very pricy thus not widely used. Same pricy issue for activated recombinant factor VII (rFVIIa, e.g., NovoSeven® RT). rFVIIa acts by forming a complex with tissue factor, which activates FX to FXa via extrinsic pathway, which then converting prothrombin into thrombin to achieve hemostasis.

The present invention in one aspect provides compositions (e.g., pharmaceutical compositions) of FX activators (e.g., RVV-X), such as RVV-X lead formulation, which are stable and biologically active after long-term storage, under accelerated or stress condition, and/or after reconstitution. The present invention in another aspect provides uses of FX activators or compositions thereof, such as any of the FX activator pharmaceutical compositions described herein, for treating a bleeding disorder (e.g., hemophilia, such as HA or HB with inhibitor) in an individual (e.g., human), which demonstrate superior therapeutic effects (e.g., high specificity, high activity, low dosage, fast onset), PK/PD profiles, and safety profiles in vivo. Such characteristics are comparable to or even better than FDA approved rFVIIa (NovoSeven® RT).

The FX activators (e.g., RVV-X, such as isolated and purified from the venom of Daboia nissellii siamensis) and pharmaceutical compositions thereof (e.g., RVV-X lead formulation) described herein, and uses thereof in treating bleeding disorders (e.g., HA or HB, with or without inhibitor) described herein, have one or more of the following superior effects: 1) they demonstrate excellent biological activity in promoting hemostasis in hemophilia A and hemophilia B patients with inhibitors (FVli inhibitor or FIX inhibitor, respectively), who are known difficult-to-treat populations; 2) they are effective in promoting hemostasis in various bleeding events, such as during surgery operation (see Examples 1-4); 3) they demonstrate comparable or even better therapeutic effects compared to FDA-approved hemostatics that are either expensive or with limited availability, for example, the therapeutic effect of RVV-X in human phase II trial (100% effective hemostasis) was found to be significantly better than that of thrombin complex concentrates (PCC; effective hemostasis rate 50%) and comparable to that of injectable recombinant human coagulation factor VIIa (e.g., NovoSeven® RT; effective hemostasis rate about 90°/), a single injection of RVV-X at low dose in KOFVIII hemophilia mice was effective in promoting hemostasis and reducing mortality, and exhibited comparable or even better therapeutic effect than NovoSeven® RT, etc.; 4) they have excellent safety profiles and are well tolerated within dose range demonstrated herein, without inducing any thromboembolic event, disseminated intravascular coagulation (DIC), serious adverse event (SAE), adverse event (AE) that leads to withdrawal during clinical trial, or Suspected Unexpected Serious Adverse Reaction (SUSAR) (see Examples 1 and 4); 5) they do not induce inhibitor (binding antibody) or neutralizing antibody against RVV-X (undesired immune responses) in vivo; 6) they have wide hemostatic applications on subjects ranging from mammal (e.g., mice) to human; 7) they exhibit excellent pharmacokinetics (PK) profiles and pharmacodynamics (PD) profiles such as fast onset of efficacy and long in vivo half-life, for example, both single- and multiple-dosing of RVV-X at low dose reached Ti₂ of 7-9 hours, C. and therapeutic indicators (e.g., thrombin generation (TG) increase, endogenous thrombin-generating potential (ETP) increase, activated partial thromboplastin time (APTT) shortening) appeared as early as 5-10 minutes after administration and lasted at least 24 hours, while the FDA-approved NovoSeven® RT only exhibits Tv₂ of 1.7-3.11 hours (US FDA NovoSeven Package Insert); 8) the long half-life and high biological activity of FX activators and pharmaceutical compositions thereof (e.g., RVV-X lead formulation) described herein allow low frequency and low dosage administration (e.g., 0.16 U/kg once every 4 hours within 4 doses, or 0.1 U/kg once every 4 hours within 6 doses, or single dose at 0.16 U/kg-0.48 U/kg) to achieve effective hemostasis, and can be administered via intravenous injection, which provides convenience and reduced cost to the patients; and 9) FX activator pharmaceutical compositions (e.g., RVV-X lead formulation) and formulation schemes described herein (e.g., lyophilized injection powder form) provide superior stability and safety profiles (e.g., compared to formulating with human serum albumin as protein stabilizer), such as stable appearance, moisture, pH, and biological activity stability, and significantly reduced aggregates and/or insoluble particles, under long-term storage condition (2-8° C.), accelerated condition (e.g., 25° C., RH 65f5%), stress condition (e.g., high temperature (40±2° C.), high humidity, or light), or after reconstitution, thereby effectively maintaining drug property, also avoiding risks of allergic reactions and/or virus contamination commonly seen when HSA is used as protein stabilizer.

Also provided are methods or making (e.g., formulation scheme) any of the FX activator pharmaceutical compositions described herein, and articles of manufacture comprising the FX activators (e.g., RVV-X) or pharmaceutical compositions thereof.

### I. Definitions

The practice of the present invention will employ, unless indicated specifically to the contrary, conventional methods of virology, immunology, microbiology, molecular biology and recombinant DNA techniques within the skills of the art, many of which are described below for the purpose of illustration. Such techniques are explained fully in the literature. See, e.g., Current Protocols in Molecular Biology or Current Protocols in Immunology, John Wiley & Sons, New York, N.Y. (2009); Ausubel el al., Short Protocols in Molecular Biology, 3rd ed., John Wiley & Sons, 1995; Sambrook and Russell, Molecular Cloning: A Laboratory Manual (3rd Edition, 2001); Maniatis et al., Molecular Cloning: A Laboratory Manual (1982); DNA Cloning: A Practical Approach, vol. I&II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984) and other like references.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of the disease. The methods of the invention contemplate any one or more of these aspects of treatment. For example, an individual is successfully “treated” if one or more symptoms associated with the disease are mitigated or eliminated, including, but are not limited to, decreasing symptoms resulting from the disease, increasing the quality of life of those suffering from the disease, decreasing the dose of other medications required to treat the disease, and/or prolonging survival of individuals.

The term “prevent,” and similar words such as “prevented,” “preventing,” “prophylaxis” etc., indicate an approach for preventing, inhibiting, or reducing the likelihood of the recurrence of, a disease or condition. It also refers to delaying the recurrence of a disease or condition or delaying the recurrence of the symptoms of a disease or condition. As used herein, “prevention” and similar words also includes reducing the intensity, effect, symptoms and/or burden of a disease or condition prior to recurrence of the disease or condition.

As used herein, “delaying” the development of a disease means to defer, hinder, slow, retard, stabilize, and/or postpone development of the disease. This delay can be of varying lengths of time, depending on the history of the disease and/or individual being treated. A method that “delays” development of a disease is a method that reduces probability of disease development in a given time frame and/or reduces the extent of the disease in a given time frame, when compared to not using the method. Such comparisons are typically based on clinical studies, using a statistically significant number of individuals.

The term “effective amount” used herein refers to an amount of an agent or a combination of agents, sufficient to treat a specified disorder, condition or disease such as ameliorate, palliate, lessen, and/or delay one or more of its symptoms. In some embodiments, an effective amount is an amount sufficient to delay disease development. In some embodiments, an effective amount is an amount sufficient to prevent or delay disease occurrence and/or recurrence. In some embodiments, an effective amount is an amount sufficient to relieve to some extent one or more of the symptoms associated with the disease. An effective amount can be administered in one or more administrations. In some embodiments, the effective amount of the drug or composition may: (i) reduce the amount and/or time of bleeding; (ii) increase coagulant activity; (iii) activate coagulation FX; (iv) increasing thrombin generation (TG) and/or endogenous thrombin-generating potential (ETP); (v) shortening activated partial thromboplastin time (APTT), prothrombin time (PT), and/or thrombin time (TT); (vi) promoting hemostasis, such as reducing bleeding time and/or amount; (vii) reducing mortality; and/or (viii) promoting wound healing: etc..

As used herein, coagulation pathway or coagulation cascade refers to the series of activation events that leads to the formation of an insoluble fibrin clot. In the coagulation cascade or pathway, an inactive protein of a serine protease (also called a zymogen) is converted to an active protease by cleavage of one or more peptide bonds, which then serves as the activating protease for the next zymogen molecule in the cascade. In the final proteolytic step of the cascade, fibrinogen is proteolytically cleaved by thrombin to fibrin, which is then crosslinked at the site of injury to form a clot.

As used herein, “hemostasis” refers to the stopping of bleeding or blood flow in an organ or body part. The term hemostasis can encompass the entire process of blood clotting to prevent blood loss following blood vessel injury to subsequent dissolution of the blood clot following tissue repair.

As used herein, “clotting” or “coagulation” refers to the formation of an insoluble fibrin clot, or the process by which the coagulation factors of the blood interact in the coagulation cascade, ultimately resulting in the formation of an insoluble fibrin clot.

As used herein, “procoagulant” refers to any substance that promotes blood coagulation.

As used herein, “anticoagulant” refers to any substance that inhibits blood coagulation.

As used herein, the term “osmolality” refers to a measure of solute concentration, defined as the number of millimole of solute (both non-ionized and ionized forms) per kg of solution. A desired level of osmolality can be achieved by addition of one or more stabilizers such as a sugar or sugar alcohol including, but not limited to, mannitol, dextrose, glucose, trehalose, and/or sucrose. Additional stabilizers that are suitable for providing osmolality are described in references such as the handbook of Pharmaceutical Excipients (Fourth Edition, Royal Pharmaceutical Society of Great Britain, Science & Practice Publishers) or Remingtons: The Science and Practice of Pharmacy (Nineteenth Edition, Mack Publishing Company).

As used herein, the terms “isosmotic” and “isotonic” are used interchangeably with the terms “substantially isosmotic,” and “substantially isotonic” and refer to formulations characterized by having an osmotic pressure that is the same as or at least substantially equivalent to the osmotic pressure of another solution, which is achieved by formulations wherein the total concentration of solutes, including both permeable and impermeable solutes, in the formulation are the same as or at least substantially equivalent to the total number of solutes in another solution. Thus, while it will be appreciated by those of skill in the art that “isosmotic” and “isotonic” formulations that are used for in vivo administration generally have an osmolality ranging from about 270 mmol/kg to about 310 mmol/kg, in the context of the high concentration, low viscosity formulations of the present embodiments, the terms “isosmotic,” “isotonic,” “substantially isosmotic,” and “substantially isotonic” are used interchangeably to refer to formulations having an osmolality ranging from about 240 mOsm/kg to about 400 mOsm/kg, or from about 270 mOsm/kg to about 370 mOsm/kg, or from about 300 mOsm/kg to about 330 mOsm/kg.

As used herein, an “individual” or a “subject” refers to an animal (e.g., mammal), including, but not limited to, human, bovine, horse, feline, canine, rodent, avian, or primate. In some embodiments, the individual is a human. In some embodiments, the individual is a mouse, rat, or monkey.

As used herein, the term “specifically binds,” “specifically recognizes,” or is “specific for” refers to measurable and reproducible interactions such as binding between a ligand and a receptor, which is determinative of the presence of the ligand in the presence of a heterogeneous population of molecules including biological molecules. For example, a ligand that specifically binds a receptor is a ligand that binds this receptor with greater affinity, avidity, more readily, and/or with greater duration than it binds other receptors. In some embodiments, the extent of binding of a ligand to an unrelated receptor is less than about 10% of the binding of the ligand to the target receptor as measured, e.g., by a radioimmunoassay (RIA). In some embodiments, a ligand that specifically binds a target receptor has an equilibrium dissociation constant (K_{d}) of ≤10⁻⁵ M, ≤10⁻⁶ M, ≤10⁻⁷ M, ≤10⁻⁸ M, ≤10⁻⁹ M, ≤10⁻¹¹ M, ≤10⁻¹¹ M, or ≤10⁻¹² M. In some embodiments, a ligand specifically binds a receptor that is conserved among the receptors from different species. In some embodiments, specific binding can include, but does not require exclusive binding. Binding specificity of a ligand can be determined experimentally by methods known in the art. Such methods comprise, but are not limited to Western blots, ELISA-, RIA-, ECL-, IRMA-, EIA-, BIACORE™-tests and peptide scans.

“Covalent bond” as used herein refers to a stable bond between two atoms sharing one or more electrons. Examples of covalent bonds include, but are not limited to, peptide bonds and disulfide bonds. As used herein, “peptide bond” refers to a covalent bond formed between a carboxyl group of an amino acid and an amine group of an adjacent amino acid. A “disulfide bond” as used herein refers to a covalent bond formed between two sulfur atoms, such as a combination of two Fc fragments by one or more disulfide bonds. One or more disulfide bonds may be formed between the two fragments by linking the thiol groups in the two fragments. In some embodiments, one or more disulfide bonds can be formed between one or more cysteines of two Fc fragments. Disulfide bonds can be formed by oxidation of two thiol groups. In some embodiments, the covalent linkage is directly linked by a covalent bond. In some embodiments, the covalent linkage is directly linked by a peptide bond or a disulfide bond.

“Percent (%) amino acid sequence identity” and “homology” with respect to a peptide or polypeptide sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

As used herein, the “C terminus” of a polypeptide refers to the last amino acid residue of the polypeptide which donates its amine group to form a peptide bond with the carboxyl group of its adjacent amino acid residue. “N terminus” of a polypeptide as used herein refers to the first amino acid of the polypeptide which donates its carboxyl group to form a peptide bond with the amine group of its adjacent amino acid residue.

An “isolated” polypeptide is one that has been identified, separated and/or recovered from a component of its production environment (e.g., natural or recombinant). Preferably, the isolated polypeptide is free of association with all other components from its production environment. Contaminant components of its production environment, such as that resulting from recombinant transfected cells, are materials that would typically interfere with research, diagnostic or therapeutic uses for the polypeptide, and may include enzymes, hormones, and other proteinaceous or non-proteinaceous solutes. In some embodiments, the polypeptide will be purified: (1) to greater than 95% by weight of polypeptides as determined by, for example, the Lowry method, and in some embodiments, to greater than 99% by weight; (2) to a degree sufficient to obtain at least 15 residues of N-terminal or internal amino acid sequence by use of a spinning cup sequenator; or (3) to homogeneity by SDS-PAGE under non-reducing or reducing conditions using Coomassie Blue or, preferably, silver stain. Isolated polypeptide includes the polypeptide in silu within recombinant cells since at least one component of the polypeptide's natural environment will not be present. Ordinarily, however, an isolated polypeptide will be prepared by at least one purification step.

An “isolated” nucleic acid molecule encoding a construct is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides described herein is in a form other than in the form or setting in which it is found in nature. Isolated nucleic acid molecules therefore are distinguished from nucleic acid encoding the polypeptides described herein existing naturally in cells. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence: or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that has the same function or biological activity as screened or selected for in the originally transformed cell are included herein.

The term “pharmaceutical formulation” of “pharmaceutical composition” refers to a preparation that is in such form as to permit the biological activity of the active ingredient to be effective, and that contains no additional components that are unacceptably toxic to a subject to which the formulation would be administered. Such formulations are sterile. A “sterile” formulation is aseptic or free from all living microorganisms and their spores.

It is understood that embodiments of the invention described herein include “consisting of” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat disease of type X means the method is used to treat disease of types other than X.

The term “about X-Y” used herein has the same meaning as “about X to about Y.”

As used herein and in the appended claims, the singular forms “a,” “or,” and “the” include plural referents unless the context clearly dictates otherwise.

### II. Pharmaceutical Compositions of Coagulation Factor X Activator

Inventors of the application discovered that when using disaccharide (e.g., sucrose and/or trehalose) as a stabilizer in combination with an amino acid buffer (e.g., arginine and/or histidine) to formulate compositions with FX activator (e.g., RVV-X) as the single active ingredient, it not only greatly improves product stability, reduces protein aggregation and insoluble particle content, but also avoids allergic reactions and potential virus contamination risks caused by using human serum albumin as the common stabilizer. During lyophilization of FX activator (e.g., RVV-X), the use of sucrose and/or trehalose as stabilizer can make the higher-order structure of FX activator more stable, prevent it from being deformed due to lyophilization, and retain the intact structure and function of FX activator in the case of low temperature freezing and dehydration, thus significantly improving the stability of FX activator.

During lyophilization of the FX activator (e.g., RVV-X) solution, the concentration of FX activator gradually increases, and the pH value of the solution may change at high protein concentrations, which may lead to the denaturation of FX activator in severe cases, resulting in FX activator inactivation. In order to ensure that the active ingredient FX activator has maximum biological activity, it is essential to control the appropriate pH range. Inventors of the application discovered that histidine and/or arginine as buffering agent not only has good buffering capacity, but also can maintain the relative stability of product pH within a certain range. Further, when such amino acid buffer (e.g., arginine and/or histidine) is used in combination with disaccharide (e.g., sucrose and/or trehalose) as a stabilizer, compared with commonly used stabilizer HSA, the formulation scheme of the present application can not only ensure the bioactivity of FX activators, but also further improve the stability of the composition.

The FX activator pharmaceutical compositions (e.g., injection powder) of the present invention have at least the following beneficial effects: 1) the FX activator pharmaceutical compositions or formulation schemes of the present invention, employ FX activator as the single active ingredient, use sucrose and/or trehalose instead of albumin (e.g., HSA) as a stabilizer, and are combined with a buffering agent of arginine and/or histidine and a surfactant, which not only significantly improve the stability of the FX activator composition product, but also avoid the potential risks caused by virus or other unknown components carried by albumin: 2) the FX activator pharmaceutical compositions or formulation schemes of the present invention, employ FX activator as the single active ingredient, thus the active ingredient and its content are clear, further, sucrose and/or trehalose has high purity and wide sources, thus carrying out long-term mass manufacture is easy, also easy to control costs and improve product quality; 3) the FX activator pharmaceutical compositions or formulation schemes of the present invention can ensure that FX activator maintains good stability in the process of preparation, transportation, and storage, the content of insoluble particles is low, and has better clinical drug safety and controllability for quality; and 4) the FX activator pharmaceutical compositions or formulation schemes of the present invention does not contain albumin and has higher stability, after reconstituted with 0.9% sodium chloride injection, it can remain stable for at least about 8 hours at room temperature.

One aspect of the present application provides pharmaceutical compositions of coagulation factor X activator (FX activator), hereinafter also referred to as “FX activator pharmaceutical composition” or “FX activator formulation.” In some embodiments, the FX activator pharmaceutical composition (e.g., lyophilized) comprises (or consists essentially of, or consists of): i) an FX activator (e.g., RVV-X) in an amount of from about 0.1 U/mL to about 200 U/mL (e.g., about 10 U/mL); ii) a stabilizer (e.g., sucrose) in an amount of from about 2 mg/ml to about 100 mg/ml (e.g., about 30 mg/ml); iii) a buffering agent (e.g., histidine) in an amount of from about 0.1 mg/ml to about 50 mg/ml (e.g., about 3 mg/ml); iv) a surfactant (e.g., polysorbate 20) in an amount of from about 0.001% (w/v) to about 0.1% (w/v) (e.g., about 0.02% (w/v)); and v) a tonicity agent (e.g., mannitol) in an amount of from about 1 mg/ml to about 100 mg/ml (e.g., about 40 mg/ml); wherein the pharmaceutical composition has a pH of from about 6.0 to about 8.0 (e.g., about 6.85). In some embodiments, the FX activator is RVV-X. In some embodiments, the RVV-X is isolated from Daboia russellii siamensis venom. In some embodiments, RVV-X comprises a) i) a heavy chain comprising the sequence of SEQ ID NO: 1, or a sequence with at least about 80% (e.g., at least about any of 85%, 88%, 90%, 95%, 96%, 97%, 98%, 99%, or more) identity to SEQ ID NO: 1; ii) a light chain 1 comprising the sequence of SEQ ID NO: 2, or a sequence with at least about 80% (e.g., at least about any of 85%, 88%, 90%, 95%, 96%, 97%, 98%, 99%, or more) identity to SEQ ID NO: 2; and iii) a light chain 2 comprising the sequence of SEQ ID NO: 3, or a sequence with at least about 80% (e.g., at least about any of 85%, 88%, 90%, 95%, 96%, 97%, 98%, 99%, or more) identity to SEQ ID NO: 3; b) i) a heavy chain comprising the sequence of SEQ ID NO: 1, or a sequence with at least about 80% (e.g., at least about any of 85%, 88%, 90°/c, 95%, 96%, 97%, 98%, 99%, or more) identity to SEQ ID NO: 1; ii) a light chain 1 comprising the sequence of SEQ ID NO: 5, or a sequence with at least about 80% (e.g., at least about any of 85%, 88%, 90%, 95%, 96%, 97%, 98%, 99%, or more) identity to SEQ ID NO: 5; and iii) a light chain 2 comprising the sequence of SEQ ID NO: 3, or a sequence with at least about 80% (e.g., at least about any of 85%, 88%, 90%, 95%, 96%, 97%, 98%, 99%, or more) identity to SEQ ID NO: 3; or c) a mixture of a) and b).

In some embodiments, there is provided an FX activator pharmaceutical composition (e.g., lyophilized) comprises (or consists essentially of, or consists of). i) an FX activator (e.g., RVV-X) in an amount of from about 1 U/mL to about 100 U/mL; ii) a stabilizer (e.g., sucrose) in an amount of from about 15 mg/ml to about 60 mg/ml; iii) a buffering agent (e.g., histidine) in an amount of from about 2 mg/ml to about 15 mg/ml; iv) a surfactant (e.g., polysorbate 20) in an amount of from about 0.005% (w/v) to about 0.05% (w/v); and v) a tonicity agent (e.g., mannitol) in an amount of from about 10 mg/ml to about 60 mg/ml; wherein the pharmaceutical composition has a pH of from about 6.0 to about 8.0. In some embodiments, the FX activator is RVV-X. In some embodiments, the RVV-X is isolated from Daboia russellii siamensis venom.

In some embodiments, there is provided an FX activator pharmaceutical composition (e.g., lyophilized) comprises (or consists essentially of, or consists of): i) an FX activator (e.g., RVV-X) in an amount of from about 5 U/mL to about 100 U/mL; ii) a stabilizer (e.g., sucrose) in an amount of from about 30 mg/ml to about 50 mg/ml; iii) a buffering agent (e.g., histidine) in an amount of from about 2 mg/ml to about 15 mg/ml; iv) a surfactant (e.g., polysorbate 20) in an amount of from about 0.01% (w/v) to about 0.05% (w/v); and v) a tonicity agent (e.g., mannitol) in an amount of from about 30 mg/ml to about 60 mg/ml; wherein the pharmaceutical composition has a pH of from about 6.3 to about 7.3. In some embodiments, the FX activator is RVV-X. In some embodiments, the RVV-X is isolated from Daboia rissellii siamensis venom.

In some embodiments, there is provided an FX activator pharmaceutical composition (e.g., lyophilized) comprises (or consists essentially of, or consists of): i) an FX activator (e.g., RVV-X) in an amount of from about 5 U/mL to about 50 U/mL; ii) a stabilizer (e.g., sucrose) in an amount of from about 30 mg/ml to about 50 mg/ml; iii) a buffering agent (e.g., histidine) in an amount of from about 3 mg/ml to about 5 mg/ml; iv) a surfactant (e.g., polysorbate 20) in an amount of from about 0.01% (w/v) to about 0.03% (w/v); and v) a tonicity agent (e.g., mannitol) in an amount of from about 30 mg/ml to about 60 mg/ml; wherein the pharmaceutical composition has a pH of from about 6.8 to about 7.0. In some embodiments, the FX activator is RVV-X. In some embodiments, the RVV-X is isolated from Daboia russellii siarensis venom.

In some embodiments, there is provided an FX activator pharmaceutical composition (e.g., lyophilized) comprises (or consists essentially of, or consists of): i) an FX activator (e.g., RVV-X) in an amount of about any of 5 U/mL, 10 U/mL, 20 U/mL, 30 U/mL, 40 U/mL, or 50 U/mL; ii) a stabilizer (e.g., sucrose) in an amount of from about 30 mg/ml to about 50 mg/ml; iii) a buffering agent (e.g., histidine) in an amount of from about 3 mg/ml to about 5 mg/ml; iv) a surfactant (e.g., polysorbate 20) in an amount of from about 0.01% (w/v) to about 0.03% (w/v); and v) a tonicity agent (e.g., mannitol) in an amount of from about 30 mg/ml to about 60 mg/ml; wherein the pharmaceutical composition has a pH of from about 6.8 to about 7.0 (e.g., about 6.85). In some embodiments, the FX activator is RVV-X. In some embodiments, the RVV-X is isolated from Daboia russellii siamensis venom.

In some embodiments, there is provided an FX activator pharmaceutical composition (e.g., lyophilized) comprises (or consists essentially of, or consists of): i) an FX activator (e.g., RVV-X) in an amount of from about 5 U/mL to about 50 U/mL; ii) a stabilizer (e.g., sucrose) in an amount of about any of 30 mg/ml, 40 mg/ml, or 50 mg/ml; iii) a buffering agent (e.g., histidine) in an amount of from about 3 mg/ml to about 5 mg/ml; iv) a surfactant (e.g., polysorbate 20) in an amount of from about 0.01% (w/v) to about 0.03% (w/v); and v) a tonicity agent (e.g., mannitol) in an amount of from about 30 mg/ml to about 60 mg/ml; wherein the pharmaceutical composition has a pH of from about 6.8 to about 7.0 (e.g., about 6.85). In some embodiments, the FX activator is RVV-X. In some embodiments, the RVV-X is isolated from Daboia russellii siamensis venom.

In some embodiments, there is provided an FX activator pharmaceutical composition (e.g., lyophilized) comprises (or consists essentially of, or consists of): i) an FX activator (e.g., RVV-X) in an amount of from about 5 U/mL to about 50 U/mL; ii) a stabilizer (e.g., sucrose) in an amount of from about 30 mg/ml to about 50 mg/ml; iii) a buffering agent (e.g., histidine) in an amount of about any of 3 mg/ml, 4 mg/ml, or 5 mg/ml; iv) a surfactant (e.g., polysorbate 20) in an amount of from about 0.01% (w/v) to about 0.03% (w/v); and v) a tonicity agent (e.g., mannitol) in an amount of from about 30 mg/ml to about 60 mg/ml; wherein the pharmaceutical composition has a pH of from about 6.8 to about 7.0 (e.g., about 6.85). In some embodiments, the FX activator is RVV-X. In some embodiments, the RVV-X is isolated from Daboia russellii siamensis venom.

In some embodiments, there is provided an FX activator pharmaceutical composition (e.g., lyophilized) comprises (or consists essentially of, or consists of): i) an FX activator (e.g., RVV-X) in an amount of from about 5 U/mL to about 50 U/mL; ii) a stabilizer (e.g., sucrose) in an amount of from about 30 mg/ml to about 50 mg/ml; iii) a buffering agent (e.g., histidine) in an amount of from about 3 mg/ml to about 5 mg/ml; iv) a surfactant (e.g., polysorbate 20) in an amount of about any of 0.01% (w/v), 0.02% (w/v), or 0.03% (w/v); and v) a tonicity agent (e.g., mannitol) in an amount of from about 30 mg/ml to about 60 mg/ml; wherein the pharmaceutical composition has a pH of from about 6.8 to about 7.0 (e.g., about 6.85). In some embodiments, the FX activator is RVV-X. In some embodiments, the RVV-X is isolated from Daboia russellii siamensis venom.

In some embodiments, there is provided an FX activator pharmaceutical composition (e.g., lyophilized) comprises (or consists essentially of, or consists of): i) an FX activator (e.g., RVV-X) in an amount of from about 5 U/mL to about 50 U/mL; ii) a stabilizer (e.g., sucrose) in an amount of from about 30 mg/ml to about 50 mg/ml; iii) a buffering agent (e.g., histidine) in an amount of from about 3 mg/ml to about 5 mg/ml; iv) a surfactant (e.g., polysorbate 20) in an amount of from about 0.01% (w/v) to about 0.03% (w/v); and v) a tonicity agent (e.g., mannitol) in an amount of about any of 30 mg/ml, 40 mg/ml, 50 mg/ml, or 60 mg/ml; wherein the pharmaceutical composition has a pH of from about 6.8 to about 7.0 (e.g., about 6.85). In some embodiments, the FX activator is RVV-X. In some embodiments, the RVV-X is isolated from Daboia russellii siamensis venom.

In some embodiments, there is provided a FX activator pharmaceutical composition (e.g., lyophilized) comprising (or consisting essentially of, or consisting of): i) RVV-X in an amount of about 10 U/mL; ii) sucrose in an amount of about 30 mg/ml; iii) histidine in an amount of about 3 mg/ml; iv) polysorbate 20 in an amount of about 0.02% (w/v); and v) mannitol in an amount of about 40 mg/ml; wherein the pharmaceutical composition has a pH of about 6.85 (hereinafter also referred to as “lead formulation” or “RVV-X lead formulation”). In some embodiments, the RVV-X is isolated from Daboia russellii siamensis venom. In some embodiments, RVV-X comprises a) i) a heavy chain comprising the sequence of SEQ ID NO: 1; ii) a light chain 1 comprising the sequence of SEQ ID NO. 2; and iii) a light chain 2 comprising the sequence of SEQ ID NO: 3; b) i) a heavy chain comprising the sequence of SEQ ID NO: 1; ii) a light chain 1 comprising the sequence of SEQ ID NO: 5; and iii) a light chain 2 comprising the sequence of SEQ ID NO: 3; or c) a mixture of a) and b).

In some embodiments, the pharmaceutical composition is a lyophilized formulation (e.g., injection powder). In some embodiments, the pharmaceutical composition is an aqueous solution.

A reconstituted formulation can be prepared by dissolving a lyophilized FX activator pharmaceutical composition in a diluent such that the protein is dispersed throughout. Exemplary pharmaceutically acceptable (safe and non-toxic for administration to a human) diluents suitable for use in the present application include, but are not limited to, sterile water, bacteriostatic water for injection (BWFI), a pH buffered solution (e.g. phosphate-buffered saline), sterile saline solution (e.g., 0.9% NaCl injection), glucose injection (e.g., 5% glucose injection), Ringer's solution or dextrose solution, or aqueous solutions of salts and/or buffers.

In some embodiments, the pharmaceutical composition is sterile. In order for the pharmaceutical compositions to be used for in vivo administration, they must be sterile. The pharmaceutical composition may be rendered sterile by filtration through sterile filtration membranes. The pharmaceutical compositions herein generally are placed into a container having a sterile access port, for example, an intravenous solution bag or vial having a stopper pierceable by a hypodermic injection needle.

The pharmaceutical compositions herein may also contain more than one active compound as necessary for the particular indication being treated, preferably those with complementary activities that do not adversely affect each other. Such molecules are suitably present in combination in amounts that are effective for the purpose intended.

In some embodiments, the pharmaceutical composition is contained in a single-use vial, such as a single-use sealed vial. In some embodiments, the pharmaceutical composition is contained in a multi-use vial. In some embodiments, the pharmaceutical composition is contained in bulk in a container. In some embodiments, the pharmaceutical composition is cryopreserved.

In some embodiments, the pharmaceutical composition is contained in a single-use vial. In some embodiments, the vial contains FX activator (e.g., RVV-X) in an amount of from about 1U to about 30U, such as any of from about 1U to about 5U, from about 1U to about 10U, from about 2U to about 18U, from about 3U to about 15U, or from about 5U to about 10U. In some embodiments, the vial contains FX activator (e.g., RVV-X) in an amount of about 5U. In some embodiments, the pharmaceutical composition (e.g., lyophilized) is reconstituted with 2 mL of 0.9% sodium chloride injection, for administration.

In some embodiments, the pharmaceutical composition comprises a homogeneous population of FX activators (e.g., RVV-X) described herein. A homogeneous population means the FX activators are exactly the same to each other. In some embodiments, at least about 70% (such as at least about any of 75%, 80%, 85%, 90%, 95%, 96%, 97%, 98%, or 99%) of the FX activators in the pharmaceutical composition are homogeneous.

In some embodiments, the pharmaceutical composition consists essentially of (or consists of) FX activators (e.g., RVV-X) described herein, and optionally a pharmaceutically acceptable carrier. In some embodiments, the pharmaceutical composition does not comprise any host cell (e.g., CHO) or non-FX activator animal (e.g., snake) protein.

### Coagulation Factor X Activator (FX Activator)

Any agent that can remove the activation peptide from the N-terminus of the FX heavy chain, hence resulting in an activated FXa form, can be used as FX activator. In some embodiments, an FX activator described herein is a proteinaceous activator, such as an enzyme with proteolytic properties that act on FX. In some embodiments, the FX activator can be non-proteinaceous activator.

In some embodiments, the FX activator is isolated from venom of snakes, such as snake species that belong to the genus Viperidae and Crotalidae as well as a few Elapid species. Also see G. Tans and J. Rosing, “Snake venom activators of factor X: an overview,” Haemostasis. 2001:31(3-6):225-33 (the content of which is incorporated herein by reference in its entirety) for any venom-derived FX activator that can be used in the present invention. In some embodiments, the FX activator is a metalloprotease. In some embodiments, the FX activator is a serine protease. In some embodiments, the FX activator is RVV-X, such as RVV-X isolated from Daboia russellii siamensis venom. In some embodiments, the FX activator can be recombinantly prepared.

In some embodiments, the FX activator is a wildtype venom-derived FX activator. In some embodiments, the FX activator is a natural variant of venom-derived FX activator. In some embodiments, the FX activator is a derivative of a venom-derived FX activator. In some embodiments, the FX activator is a functional fragment of a venom-derived FX activator. In some embodiments, the FX activator is a mutant of a venom-derived FX activator. In some embodiments, functional fragment or mutant form of venom-derived FX activator retains at least about 30% (such as at least about any of 40%, 50%, 60%, 70%, 80%, 90%, 95%, or more) activity of a wildtype venom-derived FX activator.

In some embodiments, the pharmaceutical composition (e.g., lyophilized) comprises an FX activator (e.g., RVV-X) in an amount of from about 0.1 U/mL to about 200 U/mL, such as any of from about 0.1 U/mL to about 1 U/mL, from about 1 U/mL to about 100 U/mL, from about 5 U/mL to about 100 U/mL, from about 5 U/mL to about 80 U/mL, from about 10 U/mL to about 50 U/mL, from about 1 U/mL to about 50 U/mL, from about 5 U/mL to about 50 U/mL, from about 5 U/mL to about 20 U/mL, from about 5 U/mL to about 10 U/mL, from about 1 U/mL to about 30 U/mL, or from about 5 U/mL to about 15 U/mL. In some embodiments, the pharmaceutical composition (e.g., lyophilized) comprises an FX activator (e.g., RVV-X) in an amount of about any of 0.1 U/mL, 0.5 U/mL, 1 U/mL, 5 U/mL, 10 U/mL, 20 U/mL, 30 U/mL, 40 U/mL, 50 U/mL, 60 U/mL, 70 U/mL, 80 U/mL, 90 U/mL, or 100 U/mL, such as about 10 U/mL. Also see “Bioactivity” subsection below for activity unit (U) measurement.

Coavulation Factor X (jFX) and Activated FX (Xa)

FX or FX polypeptide is a serine protease polypeptide that exhibits catalytic activity against prothrombin (i.e., prothrombogenic activity) when in active form. FX is a serine protease that is part of the coagulation pathway, and specifically is the first serine protease in the common coagulation pathway. FX is processed in cells from a precursor polypeptide to yield a polypeptide containing a propeptide region, which is eventually cleaved to generate a mature FX polypeptide lacking the signal sequence and propeptide. The secreted FX polypeptide is a two-chain polypeptide. The active FXa lacks the activation peptide. FXa is the form of FX that exhibits catalytic activity, which is increased greatly upon binding of active FX (FXa) to its cofactor Factor Va. FXa activity also is enhanced by the inclusion of Ca²⁺ and phospholipid. Mutations can be introduced that result in conformational changes of a FXa form to a zymogen-like form, that when in fully active form in the presence of FVa cofactor, exhibits catalytic activity against prothrombin. Reference to FX or FX polypeptides herein includes all forms, which include precursor, single single-chain and two-chain forms thereof, including mature forms, zymogen forms.

FX can be derived from any organism, such as mammals, including, but are not limited to, livestock animals (e.g., cows, sheep, goats, cats, dogs, donkeys, and horses), primates (e.g., human and non-human primates such as monkeys or chimpanzees), rabbits, and rodents (e.g., mice, rats, gerbils, and hamsters). FX can also be prepared by recombinant or synthetic methods. In some embodiments, FX is a recombinant FX (rFX), such as recombinant murine (rmFX) or human FX (rhFX).

In some embodiments, FX is human FX polypeptides (such as wildtype human FX), including the precursor polypeptide (488 aa), and single-chain and two-chain forms thereof, including mature forms (448 aa), zymogen forms. The human zymogen form thereof is a two-chain form containing a 139 amino acid light chain and a 306 amino acid heavy chain. The human FXa form thereof is a two-chain form containing a 139 amino acid light chain and a 254 amino acid heavy chain.

Reference to FX also includes variants thereof, such as allelic variants and species variants, variants encoded by splice variants, and other variants, including polypeptides that have at least about any of 40%, 45%, 50%, 55%, 65%, 70%, 75%, 80%, 85%, 90%, 95%, 96%, 97%, 98%, 99% or more sequence identity to the precursor polypeptide (e.g., human precursor FX) or the mature form, zymogen form. Such variants exhibit one or more FX activities including, but not limited to, FVa binding, catalytic activity, prothrombin binding, prothrombinase activity and/or coagulant activity. The activity can be reduced or increased compared to the activity of a native or wildtype FX. For example, FX polypeptide variants exhibit at least about any of 20%, 30%, 40%, 50%, 60%, 70%, 90%, 90%, 100%, 110%, 120%, 130%, 140%, 150%, 160%, 170%, 180%, 190%, 200% or more the activity of a native or wildtype FX polypeptide.

A precursor FX polypeptide refers to a non-secreted form of a FX polypeptide that contains an N-terminal signal peptide that targets the protein for secretion and a propeptide. The signal peptide is cleaved off in the endoplasmic reticulum.

A “proregion,” “propeptide,” or “pro-sequence,” refers to a region or a segment that is cleaved to produce a mature protein. A proregion is a sequence of amino acids positioned at the amino terminus of a mature biologically active polypeptide and can be as little as a few amino acids or can be a multidomain structure. For FX polypeptides, the propeptide region is generally about 9 amino acids, but can vary (e.g. longer or shorter) depending on species. For FX, the propeptide sequence functions in post-translational modification of the protein and is cleaved prior to secretion of the protein from the cell. For example, the propeptide is the recognition element for γ-carboxylation by the vitamin K-dependent carboxylase in the endoplasmic reticulum. The reaction occurs by conversion of glutamic acid residues in the Gla domain of FX to γ-carboxyglutamic acid (Gla). This modification is required for optimal Ca²⁺ mediated activation of the zymogen in the blood. For example, the Gla residues permit factor X/Xa to bind phospholipid (i.e. cell surfaces) in a calcium dependent manner, which is a requirement for assembly of the prothrombinase complex.

A propeptide form of FX is a protein that lacks the signal peptide, but retains the propeptide.

A “mature FX polypeptide” refers to a FX polypeptide that lacks a signal sequence and a propeptide sequence. The propeptide is removed by proteolytic cleavage in the trans-Golgi apparatus prior to secretion of the polypeptide. The mature FX polypeptide generally refers to a single chain form of FX prior to intrachain proteolysis to generate a two-chain polypeptide.

An activation peptide is a segment present at the N-terminus of the FX heavy chain that functions to suppress proteolytic activity by masking the catalytic machinery and thus preventing formation of the catalytic intermediate (i.e., by conformationally occluding the substrate binding site). Exemplary of an FX activation peptide is the 52 amino acid residue activation peptide at the N-terminus of the heavy chain of the mature human FX polypeptide.

“Wild-type” or “native” with reference to FX refers to a FX polypeptide encoded by a native or naturally occurring FX gene, including allelic variants, that is present in an organism, including human and other animals, in nature. Where reference is made to a wildtype or native FXa polypeptide, it is understood that this is the active or catalytically active portion of the FXa polypeptide. Reference to wild-type factor X without reference to a species is intended to encompass any species of a wild-type factor X. Included among wild-type FX polypeptides are the encoded precursor polypeptide, fragments thereof, and processed forms thereof, such as a mature form lacking the signal peptide and propeptide, as well as any pre- or post-translationally processed or modified forms thereof. Also included among native FX polypeptides are those that are post-translationally modified, including, but not limited to, modification by glycosylation, carboxylation and hydroxylation. Native FX polypeptides also include two-chain secreted forms, including the zymogen and active forms, as well as any processed forms or isoforms thereof. For example, humans express native FX.

Species variants refer to variants in polypeptides among different species, including different mammalian species, such as mouse and human. Allelic variants refer to variations in proteins among members of the same species. A splice variant refers to a variant produced by differential processing of a primary transcript of genomic DNA that results in more than one type of mRNA.

Factor X circulates in blood as an inactive precursor, called a zymogen, and requires proteolytic cleavage for activation. Zymogens possesses about 10,000-fold or less proteolytic activity when compared to the serine protease produced following activation.

An active FX (FXa) or activated FX refers to a two-chain form of a FX polypeptide, whereby the heavy chain does not contain an N-terminal activation peptide. FXa is activated by cleavage of the heavy chain to remove the activation peptide. Hence, FXa is a heterodimer that is composed of 2 chains joined by a disulfide bond. For human FX, the light chain has a molecular weight of about 16,000 daltons (139 aa), and heavy chain has a molecular weight of about 38,000 daltons (254 aa) that make up the serine protease domain. Activation of human FX occurs by cleavage of the Arg 194-Ile195 bond, which releases the activation peptide. Activation is achieved by the extrinsic Factor Xase complex (factor VIIa/TF complex) or the intrinsic Factor Xase complex (FIXa/FVIIIa complex). Activation generally requires the presence of phospholipid and calcium ions. Activation also can be achieved by Russell's viper venom (RVV-X). FXa exhibits catalytic activity, FVa binding, heparin binding, prothrombin binding, prothrombinase activity and/or coagulant activity. For purposes herein, reference to FXa refers to any FX two-chain form that lacks the activation peptide and that is capable of exhibiting FXa activities such as catalytic activity, FVa binding, heparin binding, prothrombin binding, prothrombinase activity and/or coagulant activity. Reference to FXa includes zymogen-like FXa polypeptides that, in the presence of saturating concentrations of FVa, exhibit FXa activities.

A “catalytically active portion” of a FXa polypeptide refers to an active FXa polypeptide that contains a contiguous portion of amino acids of the heavy chain that includes the catalytic triad residues, but does not include the full-sequence of the heavy chain. The catalytically active portion also can contain all or a portion of the light chain of FXa. The catalytically active portion of a FXa polypeptide exhibits at least about any of 1%, 2%, 3%, 4%, 5%, 6%, 7%, 8%, 9%, 10%, 20%, 30%, 40%, 50%, 60%, 70%, 80%, 90%, or more of the activity, such as at least about any of 120%, 130%, 140%, 150%, 200%, 300%, 400%, 5000% or more of the activity, compared to the full-length FXa. It is understood that reference herein to a modified FXa or catalytically active portion thereof means that the catalytically active portion contains the modification(s) (e.g. amino acid replacement(s)).

While FXa has the potential to serve as a therapeutic procoagulant that bypasses the deficiencies in other clotting factors in the cascade, direct use of fully functional FX as a therapeutic has proven to be impractical due to excessive activation of systemic coagulation. Another limitation of directly using FXa as a therapeutic resides in the short half-life of circulating FXa due to rapid inactivation thereof by plasma protease inhibitors, such as antithrombin (AT) III and Protein C.

### Russell's Viper Venom (RVV-X)

Russell's viper venom (RVV-X) is a venom metalloprotease of Russell's viper, or Daboia russelli, or Vipera russe/li (e.g. Daboia russeiii siamensis, Daboia russe/lii nssellii) that specifically activates factor X (Takeya et al. (1992). J. Biol. Chem., 267:14109-14117. RVV-X has a molecular weight of about 79,000 daltons and contains a disulfide bonded heavy and light chain. RVV-X does not require phospholipids for factor X activation, but does require exogenous Ca²⁺ and the presence of the amino terminal Gla domain for enhanced activation. The activity of RVV-X is inhibited in the presence of EDTA. Purified preparations of RVV-X are known and available (see, e.g., Catalog No. RVVX-2010, Haematologic Technologies, Inc.; Catalog No. ab62233, Abcam, Cambridge, Mass.), and its sequence is known (see, e.g., Takeya et al. (1992) J. Biol. Chem., 267:14109-14117 and Uniprot No. Q7LZ61, Q4PRD1 and Q4PRD2). RVV-X binds to the Gla domain of FX, and cleaves the heavy chain of FX. Metalloproteinase FX activators from other venoms may share similar catalytic mechanisms in view of their similar structures to RVV-X.

RVV-X comprise 3 polypeptide chains: a heavy chain containing the catalytic domain, and two light chains that share homology with C-type lectins and are thought to exert a regulatory function in Ca²⁺-dependent activation of FX. In some embodiments, RVV-X comprises a) i) a heavy chain (RVV-XH or a chain) comprising the sequence of SEQ ID NO: 1, or a sequence with at least about 80% (e.g., at least about any of 85%, 88%, 90%, 95%, 96%, 97%, 98%, 99%, or more) identity to SEQ ID NO: 1; ii) a light chain 1 (RVV-XL1 or γ chain) comprising the sequence of SEQ ID NO: 2, or a sequence with at least about 80% (e.g., at least about any of 85%, 88%, 90%, 95%, 96%, 97%, 98%, 99%, or more) identity to SEQ ID NO: 2; and iii) a light chain 2 (RVV-XL2 or β chain) comprising the sequence of SEQ ID NO: 3, or a sequence with at least about 80% (e.g., at least about any of 85%, 88%, 90%, 95%, 96%, 97%, 98%, 99%, or more) identity to SEQ ID NO: 3; b) i) a heavy chain comprising the sequence of SEQ ID NO: 1, or a sequence with at least about 80% (e.g., at least about any of 85%, 88%, 90%, 95%, 96%, 97%, 98%, 99%, or more) identity to SEQ ID NO: 1; ii) a light chain 1 comprising the sequence of SEQ ID NO: 5, or a sequence with at least about 80% (e.g., at least about any of 85%, 88%, 90%, 95%, 96%, 97%, 98%, 99%, or more) identity to SEQ ID NO: 5; and iii) a light chain 2 comprising the sequence of SEQ ID NO: 3, or a sequence with at least about 80% (e.g., at least about any of 85%, 88%, 90%, 95%, 96%, 97/o, 98%, 99%, or more) identity to SEQ ID NO: 3; or c) a mixture of a) and b). In some embodiments, RVV-X comprises a) i) a heavy chain comprising the sequence of SEQ ID NO: 1; ii) a light chain 1 comprising the sequence of SEQ ID NO: 2; and iii) a light chain 2 comprising the sequence of SEQ ID NO: 3; b) i) a heavy chain comprising the sequence of SEQ ID NO: 1; ii) a light chain 1 comprising the sequence of SEQ ID NO: 5; and iii) a light chain 2 comprising the sequence of SEQ ID NO: 3; or c) a mixture of a) and b). When RVV-X comprises a mixture of SEQ ID NO: 2 or 5 (or a sequence with at least about 80% identity to SEQ ID NO: 2 or 5) for the γ chain, the γ chain of SEQ ID NO: 2 (or a sequence with at least about 80% identity to SEQ ID NO: 2) and the γ chain of SEQ ID NO: 5 (or a sequence with at least about 80% identity to SEQ ID NO: 5) can be of any ratio, such as any of 99.9:0.1, 99:1, 98:2, 97:3, 96:4, 95:5, 90:10, 85:15, 80:20, 75:25, 70:30, 65:35, 60:40, 55:45, 50:50, 45:55, 40:60, 35:65, 30:70, 25:75, 20:80, 15:85, 10:90, 5:95, 1:99, 0.1:99.9, or any ratios in between. In some embodiments, RVV-X comprises (consists essentially of, or consists of) i) a heavy chain comprising the sequence of SEQ ID NO: 1; ii) a light chain 1 comprising the sequence of SEQ ID NO: 2; and iii) a light chain 2 comprising the sequence of SEQ ID NO: 3.

In some embodiments, the RVV-X is a wildtype RVV-X. In some embodiments, the RVV-X is an RVV-X natural variant. In some embodiments, the RVV-X is an analog of an RVV-X, such as an RVV-X comprising no more than about 5 amino acids (such as 4, 3, 2, or 1 aa) mutation sites. In some embodiments, the RVV-X is a derivative of an RVV-X. In some embodiments, the RVV-X is glycosylated. In some embodiments, the RVV-X is not glycosylated.

As used herein, the term “a derivative of an RVV-X” refers to a molecule having an amino acid sequence of an RVV-X or an analog of the RVV-X, but also having an additional chemical modification at one or more of the amino acid side groups, a carbon atoms, terminal amino groups, or terminal carboxyl groups. The chemical modifications include, but are not limited to, adding chemical moieties, creating new bonds, and removing chemical moieties. The modification at amino acid side groups includes, but is not limited to, an acylation of an epsilon amino group of lysine, an N-alkylation of arginine, histidine or lysine, an alkylation of carboxyl of glutamic acid or aspartic acid, and a deamination of glutamine or asparagine. The modification at terminal amino groups includes, but is not limited to, deamination, N-lower alkyl, N-di-lower alkyl, and N-acyl modifications. The modification at terminal carboxyl groups includes, but is not limited to, amide, lower alkyl acyl, dialkyl amide, and lower alkyl ester modifications. In some embodiments, the lower alkyl group is a C₁-C₄ alkyl group. In addition, one or more side groups or terminal groups may be protected by a protecting group known to a person skilled in the field of chemistry. An alpha carbon of an amino acid may be mono- or di-methylated.

In some embodiments, the RVV-X is a modified RVV-X, such as a pegylated RVV-X, or covalently modified RVV-X.

Reference to RVV-X also includes a functional fragment of a wildtype RVV-X. In some embodiments, the RVV-X is a mutant RVV-X, such as comprising one or more mutations (e.g., amino acid insertion, deletion, substitution, truncation) at one or more polypeptide chains of a wildtype RVV-X. When used as an FX activator herein, functional fragment or mutant form of wildtype RVV-X retains at least about 30% (such as at least about any of 40%, 50%, 60%, 70%, 80%, 90%, 95%, or more) activity of a wildtype RVV-X in activating FX.

Amino acid sequence variants of an FX activator (e.g., RVV-X) described herein may be prepared by introducing appropriate modifications into the nucleic acid sequence encoding the protein, or by peptide synthesis. Such modifications include, for example, deletions from, and/or insertions into and/or substitutions of residues within the amino acid sequences of the FX activator (e.g., RVV-X). Any combination of deletion, insertion, and substitution can be made to arrive at the final construct, provided that the final construct possesses the desired characteristics, e.g., retained/improved (e.g., increased at least about 1.2-fold) FX activation, retained/enhanced half-life, reduced (e.g., reduced at least about 1.2-fold) FX activation (such as to reduce excessive activation of systemic coagulation), etc.

In some embodiments, the FX activator (e.g., RVV-X) has one or more conservative amino acid substitutions. “Conservative substitution” refers to the substitution of another amino acid with the same net charge and approximately the same size and shape as the substituted amino acid. When the total number of carbon atoms and heteroatoms on their side chains differ by no more than 4, amino acids with aliphatic or substituted aliphatic amino acid side chains are roughly the same size. When the number of branches on their side chains does not differ by more than one, amino acids have roughly the same shape. Amino acids having a phenyl or substituted phenyl group on the side chain can be considered to be approximately the same in size and shape. Unless otherwise specified, natural amino acids are preferably used for conservative substitutions.

Conservative substitutions are shown in Table A. More substantial changes are provided in Table A under the heading of “exemplary substitutions,” and as further described below in reference to amino acid side chain classes. Amino acids may be grouped according to common side-chain properties: (1) hydrophobic: Norleucine, Met, Ala, Val, Leu, Ile; (2) neutral hydrophilic: Cys, Ser, Thr, Asn, Gin; (3) acidic: Asp, Glu; (4) basic: His, Lys, Arg; (5) residues that influence chain orientation: Gly, Pro; and (6) aromatic: Trp, Tyr, Phe. Non-conservative substitutions will entail exchanging a member of one of these classes for another class. Amino acid substitutions may be introduced into the protein constructs and the products screened for a desired activity mentioned above.

TABLE A
Amino acid substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn: Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

“Amino acid” is used herein in its broadest sense, including both naturally occurring amino acids and non-naturally occurring amino acids, including amino acid analogs and derivatives. The latter includes molecules that contain amino acid moieties. Those skilled in the art will realize that according to this broad definition, amino acids herein include, for example, naturally occurring L-amino acids that form proteins; D-amino acids; chemically modified amino acids, such as amino acid analogs and derivatives; naturally occurring amino acids that do not form protein, such as norleucine, β-alanine, ornithine, GABA, etc.; and chemically synthesized compounds with amino acid characteristics known in the art. The term “protein-forming” as used herein refers to amino acids that can be incorporated into peptides, polypeptides or proteins of cells through metabolic pathways.

Insertion of non-naturally occurring amino acids, including synthetic non-natural amino acids, substituted amino acids, or one or more D-amino acids, into the FX activators (e.g., RVV-X) of the present invention can have multiple benefits. D-amino acid-containing peptides and the like exhibit increased stability in vitro or in vivo compared to their counterparts containing L-amino acid. Therefore, when greater intracellular stability is desired, the construction of peptides, such as by incorporation of D-amino acids, is particularly useful. Particularly, D-peptide and the like are resistant to endogenous peptidase and protease activity, thereby improving the bioavailability of the molecule and extending the lifespan in vivo when needed. In addition, D-peptide and the like cannot be effectively processed for limited presentation by type II major histocompatibility complexes (MHC) to T helper cells, so less prone to induce humoral immune responses in the subject.

In some embodiments, the FX activator (e.g., RVV-X) is expressed recombinantly, such as in prokaryotic cells (e.g., E. coli) or in eukaryotic cells (e.g., CHO cells).

In some embodiments, the RVV-X is isolated and/or purified from Daboia russellii siamensis venom. In some embodiments, the RVV-X is isolated and/or purified as described in CN109943554B (the content of which is incorporated herein by reference in its entirety). In some embodiments, the RVV-X is sterilized and/or inactivated for virus. In some embodiments, purification of the RVV-X comprises one or more of viral inactivation, anion exchange chromatography, ultrafiltration, hydroxyapatite (HA) chromatography (HAC), and size-exclusion chromatography (SEC). In some embodiments, the purity of the RVV-X after isolation and/or purification is at least about 95% (such as at least about any of 96%, 96.5%, 97%, 97.5%, 98%, 98.5%, 99%, 99.5%, or 100%). For example, in some embodiments, the purity of the RVV-X after isolation and/or purification is at least about 98% measured by high-performance liquid chromatography (HPLC). In some embodiments, the purity of the RVV-X after isolation and/or purification is at least about 98% (e.g., 100%) measured by SDS-PAGE. In some embodiments, the bioactivity of the RVV-X after isolation and/or purification is at least about 2×10⁴ U/mg (such as at least about any of 2.5×10⁴ U/mg, 2.7×10⁴ U/mg, 2.9×10⁴ U/mg, 3×10⁴ U/mg, 4×10⁴ U/mg, 5×10⁴ U/mg, 1×10⁵ U/mg, 1×10⁶ U/mg, or more).

### Stabilizer

In some embodiments, the pharmaceutical composition (e.g., lyophilized) comprises a stabilizer (e.g., sucrose) in an amount of from about 0.1 mg/ml to about 100 mg/ml, such as any of from about 0.1 mg/ml to about 1 mg/ml, from about 1 mg/ml to about 100 mg/ml, from about 2 mg/ml to about 100 mg/ml, from about 2 mg/ml to about 60 mg/ml, from about 10 mg/ml to about 100 mg/ml, from about 1 mg/ml to about 80 mg/ml, from about 10 mg/ml to about 80 mg/ml, from about 10 mg/ml to about 70 mg/ml, from about 10 mg/ml to about 60 mg/ml, from about 1 mg/ml to about 60 mg/ml, from about 1 mg/ml to about 50 mg/ml, from about 10 mg/ml to about 50 mg/ml, from about 10 mg/ml to about 40 mg/ml, from about 10 mg/ml to about 30 mg/ml, from about 15 mg/ml to about 50 mg/ml, from about 20 mg/ml to about 60 mg/ml, from about 15 mg/ml to about 60 mg/ml, from about 30 mg/ml to about 50 mg/ml, from about 25 mg/ml to about 50 mg/ml, or from about 20 mg/ml to about 40 mg/ml. In some embodiments, the pharmaceutical composition (e.g., lyophilized) comprises a stabilizer (e.g., sucrose) in an amount of about any of 0.1 mg/ml, 0.5 mg/ml, 1 mg/ml, 5 mg/ml, 10 mg/ml, 20 mg/ml, 25 mg/ml, 30 mg/ml, 40 mg/ml, 50 mg/ml, 60 mg/ml, 70 mg/ml, 80 mg/ml, 90 mg/ml, or 100 mg/ml. In some embodiments, the pharmaceutical composition (e.g., lyophilized) comprises a stabilizer (e.g., sucrose) in an amount of about any of 25 mg/ml, 30 mg/ml, 40 mg/ml, or 50 mg/ml, such as about 30 mg/ml.

In some embodiments, the pharmaceutical composition (e.g., lyophilized) comprises a stabilizer (e.g., sucrose) in an amount of from about 0.1 mM to about 300 mM, such as any of from about 0.1 mM to about 1 mM, from about 1 mM to about 300 mM, from about 1 mM to about 200 mM, from about 1 mM to about 100 mM, from about 10 mM to about 300 mM, from about 10 mM to about 200 mM, from about 10 mM to about 100 mM, from about 50 mM to about 300 mM, from about 50 mM to about 200 mM, from about 50 mM to about 100 mM, from about 50 mM to about 90 mM, or from about 70 mM to about 90 mM. In some embodiments, the pharmaceutical composition (e.g., lyophilized) comprises a stabilizer (e.g., sucrose) in an amount of about 87.64 mM.

In some embodiments, the stabilizer comprises or is disaccharide. In some embodiments, the stabilizer is a mixture of sucrose and trehalose. In some embodiments, the stabilizer is trehalose. In some embodiments, the stabilizer is sucrose. In some embodiments, the stabilizer is in anhydrous form. In some embodiments, the stabilizer is in hydrated form, such as dihydrate form.

In some embodiments, stabilizers can protect proteins against denaturation as a result of lyophilization. In some embodiments, stabilizers are used in the lyophilization process, and the resulting lyophilates and compositions produced thereby comprise the same. In some embodiments, stabilizers enable the pharmaceutical composition to resist degradation when exposed to stress, such as elevated temperatures and/or radiation. In some embodiments, the stabilizer may enhance the ability of the pharmaceutical composition to dissolve when the pharmaceutical composition is applied to the injury or bleeding site. In some embodiments, the stabilizer enhances the usable shelf life of the pharmaceutical composition. In certain embodiments, the stabilizer provides the pharmaceutical composition with a shelf life of at least about 3 months (e.g., at least about any of 6 months, 1 year, 2 years, 3 years or longer). As used herein, the term usable shelf life means that the pharmaceutical composition does not exhibit noticeable degradation when viewed without magnification or with magnification such as a magnifying glass or microscope.

Any agent that can improve the stability and/or prolong the shelf life of the pharmaceutical formulation described herein can be used as stabilizer. Examples of stabilizers include, but are not limited to, glycine, alanine, glutamate, methionine, arginine, benzoic acid, citric, glycolic, lactic, malic, maleic acid, polyol (such as sorbitol, mannitol, and trehalose), human serum, albumin (HSA), sugar (such as glucose, fructose, galactose, maltose, lactose, and sucrose), ethylenediaminetetraacetic acid (EDTA), diethylenetriaminepentaacetic acid (DTPA), hydroxyethylenediaminetriacetic acid (HEDTA), ethylene glycol-bis-(2-aminoethyl)-N, N, N′,N′-tetraacetic acid (EGTA), nitrilotriacetic acid (NTA), metal ion stabilizing agent, and citrates.

### Buffering Agent

In some embodiments, the pharmaceutical composition (e.g., lyophilized) comprises a buffering agent (e.g., histidine) in an amount of from about 0.1 mg/ml to about 50 mg/ml, such as any of from about 0.1 mg/ml to about 1 mg/ml, from about 0.1 mg/ml to about 5 mg/ml, from about 1 mg/ml to about 50 mg/ml, from about 1 mg/ml to about 40 mg/ml, from about 1 mg/ml to about 30 mg/ml, from about 2 mg/ml to about 20 mg/ml, from about 1 mg/ml to about 10 mg/ml, from about 2 mg/ml to about 15 mg/ml, from about 1 mg/ml to about 8 mg/ml, from about 3 mg/ml to about 5 mg/ml, from about 2.5 mg/ml to about 5 mg/ml, or from about 1 mg/ml to about 5 mg/ml. In some embodiments, the pharmaceutical composition (e.g., lyophilized) comprises a buffering agent (e.g., histidine) in an amount of about any of 0.1 mg/ml, 0.5 mg/ml, 1 mg/ml, 2 mg/ml, 2.5 mg/ml, 3 mg/ml, 4 mg/ml, 5 mg/ml, 10 mg/ml, 20 mg/ml, 30 mg/ml, 40 mg/ml, or 50 mg/ml. In some embodiments, the pharmaceutical composition (e.g., lyophilized) comprises a buffering agent (e.g., histidine) in an amount of about any of 2.5 mg/ml, 3 mg/ml, 4 mg/ml, or 5 mg/ml, such as about 3 mg/ml.

In some embodiments, the pharmaceutical composition (e.g., lyophilized) comprises a buffering agent (e.g., histidine) in an amount of from about 0.5 mM to about 350 mM, such as any of from about 0.5 mM to about 1 mM, from about 1 mM to about 300 mM, from about 1 mM to about 200 mM, from about 1 mM to about 100 mM, from about 1 mM to about 50 mM, from about 10 mM to about 50 mM, from about 50 mM to about 300 mM, from about 100 mM to about 300 mM, from about 5 mM to about 40 mM, from about 5 mM to about 30 mM, from about 10 mM to about 30 mM, or from about 10 mM to about 20 mM. In some embodiments, the pharmaceutical composition (e.g., lyophilized) comprises a buffering agent (e.g., histidine) in an amount of about 19.33 mM.

Buffering agent(s) or buffer(s) in accordance with this aspect of the invention are compatible with the protein appropriate (e.g., RVV-X) to the desired end use, provide adequate buffering capacity at concentrations consistent with acceptable osmolality, are inert, stable, and have their maximum buffering capacity at or near the desired pH.

A variety of buffering agents can be used in the lyophilization solution in accordance with various aspects and embodiments of the invention in this regard. Some of the same buffers can be used to reconstitute lyophilate. Buffering agent may include, but are not limited to, citrates, citrate-phosphates, phosphates, acetates, succinates, tartrates, maleates, HEPES, Tris, Bicine, glycine, N-glycylglycine, carbonates, glycylglycine, lysine, arginine, histidine, and/or mixtures thereof. In some embodiments, the buffering agent is sodium acetate. In some embodiments, the buffering agent comprises one or both of histidine and arginine. In some embodiments, the buffering agent is histidine.

Buffers in accordance with the aspect of the invention are effective to maintain appropriate pH. The exact optimal pH will vary from protein to protein. Accordingly, different buffer systems will be more or less better than one another for different proteins. Generally, however, the preferred buffers are effective for pH in the range of 5 to 8, or 6 to 8, especially in the range of 5.5 to 7.5.

### Surfactant

In some embodiments, the pharmaceutical composition (e.g., lyophilized) comprises a surfactant (e.g., polysorbate 20) in an amount of from about 0.001% (w/v) to about 0.1% (w/v), such as any of from about 0.001% (w/v) to about 0.01% (w/v), from about 0.005% (w/v) to about 0.05% (w/v), from about 0.005% (w/v) to about 0.01% (w/v), from about 0.01% (w/v) to about 0.1% (w/v), from about 0.01% (w/v) to about 0.05% (w/v), or from about 0.01% (w/v) to about 0.03% (w/v). In some embodiments, the pharmaceutical composition (e.g., lyophilized) comprises a surfactant (e.g., polysorbate 20) in an amount (w/v) of about any of 0.001%, 0.005%, 0.01%, 0.02%, 0.03%, 0.04%, 0.05%, 0.06%, 0.07%, 0.08%, 0.09%, or 0.1%. In some embodiments, the pharmaceutical composition (e.g., lyophilized) comprises a surfactant (e.g., polysorbate 20) in an amount of about any of 0.01% (w/v), 0.02% (w/v), or 0.03% (w/v), such as about 0.02% (w/v).

Surfactant can stabilize the formulation during processing and storage by reducing interfacial interaction and prevent protein from adsorption. Surfactant can help solubilize the proteins as well as to protect the proteins against agitation-induced aggregation, which also permits the formulation to be exposed to shear surface stress without causing denaturation of the active proteins. Any known surfactant can be used herein. In some embodiments, the surfactant comprises one or both of polysorbate and poloxamer. In some embodiments, the surfactant is selected from one or more of polysorbate 20, polysorbate 80, and poloxamer 188. In some embodiments, the surfactant is polysorbate 20 (PS20).

In some embodiments, the surfactant is a non-ionic surfactant. Non-ionic surfactants include, but not limited to, polysorbates, such as polysorbate (Tween®) 20, polysorbate (Tween®) 80, polysorbate (Tween®) 40, polysorbate (Tween®) 60; polyoxamers, such as poloxamer 184 and 188; Pluronic® polyols; spans (e.g., sorbitan); ethoxylates; fatty alcohol ethoxylates; alkylphenol ethoxylates (e.g., Triton X-100); PLURONIC® polyols; fatty acid ethoxylates; lauromacrogol 400; polyoxyl 40 stearate; polyoxyethylene hydrogenated castor oil 10, 50 and 60; glycerol monostearate; sucrose fatty acid ester; methyl cellulose; carboxymethyl cellulose; and other ethylene/polypropylene block polymers. Non-ionic surfactants can be present in a range of about 0.05 mg/ml to about 1.0 mg/ml, preferably about 0.07 mg/ml to about 0.2 mg/ml.

In some embodiments, the surfactant is an ionic surfactant, such as anionic surfactant (e.g., sodium dodecyl sulfate (SDS), dioctyle sodium sulfosuccinate, dioctyl sodium sulfonate) or cationic surfactant (e.g., benzalkonium chloride, benzethonium chloride).

During the freeze-drying process of FX activator composition, surfactant can not only reduce the freezing and dehydration deformation caused by the ice-water interfacial tension during the freezing and dehydration process, but also serve as wetting agent and heavy crease agent for the active component during the rehydration process. Inventors of the present invention discovered that using at least one of polysorbate 20, polysorbate 80, and poloxamer 188 as the surfactant not only involves lower toxicity, but also can effectively inhibit the FX activator from surface adsorption and aggregation under various conditions, thus improving the stability of the composition.

### Tonicity Agent

In some embodiments, the pharmaceutical composition (e.g., lyophilized) comprises a tonicity agent (e.g., mannitol) in an amount of from about 1 mg/ml to about 100 mg/ml, such as any of from about 1 mg/ml to about 50 mg/ml, from about 10 mg/ml to about 80 mg/ml, from about 10 mg/ml to about 60 mg/ml, from about 5 mg/ml to about 50 mg/ml, from about 20 mg/ml to about 50 mg/ml, from about 30 mg/ml to about 60 mg/ml, or from about 30 mg/ml to about 50 mg/ml. In some embodiments, the pharmaceutical composition (e.g., lyophilized) comprises a tonicity agent (e.g., mannitol) in an amount of about any of 1 mg/ml, 5 mg/ml, 10 mg/ml, 20 mg/ml, 30 mg/ml, 40 mg/ml, 50 mg/ml, 60 mg/ml, 70 mg/ml, 80 mg/ml, 90 mg/ml, or 100 mg/ml. In some embodiments, the pharmaceutical composition (e.g., lyophilized) comprises a tonicity agent (e.g., mannitol) in an amount of about any of 30 mg/ml, 40 mg/ml, 50 mg/ml, or 60 mg/ml, such as about 40 mg/ml. In some embodiments, the tonicity agent is mannitol.

In some embodiments, the pharmaceutical composition (e.g., lyophilized) comprises a tonicity agent (e.g., mannitol) in an amount of from about 5 mM to about 550 mM, such as any of from about 5 mM to about 50 mM, from about 10 mM to about 500 mM, from about 50 mM to about 400 mM, from about 50 mM to about 300 mM, from about 100 mM to about 500 mM, from about 100 mM to about 400 mM, from about 100 mM to about 300 mM, from about 150 mM to about 400 mM, from about 150 mM to about 300 mM, from about 180 mM to about 250 mM, or from about 200 mM to about 220 mM. In some embodiments, the pharmaceutical composition (e.g., lyophilized) comprises a tonicity agent (e.g., mannitol) in an amount of about 219.58 mM.

The pharmaceutical composition described herein comprises one or more tonicity agents. Tonicity agents reduce local irritation caused by pharmaceutical formulations/compositions by preventing osmotic shock at the site of application. Tonicity agents can be present to adjust or maintain the tonicity of liquid in a composition. When used with large, charged biomolecules such as proteins, they can interact with the charged groups of the amino acid side chains, to lessen the potential for inter and intra-molecular interactions. Tonicity agents can be present in any amount between 0.1% to 25% by weight, preferably 1% to 5%, taking into account the relative amounts of the other ingredients. Examples of tonicity agents include, but are not limited to, a sugar alcohol or polyol (such as mannitol or sorbitol), a non-ionic surfactant (such as polysorbate 20, polysorbate 40, polysorbate 60, polysorbate 80), and a sugar (such as sucrose). Preferred tonicity agents include polyhydric sugar alcohols, preferably trihydric or higher sugar alcohols, such as glycerin, erythritol, arabitol, xylitol, sorbitol and mannitol.

pH

In some embodiments, the pharmaceutical composition has a pH of from about 6.0 to about 8.0, such as any of from about 6.3 to about 7.3, from about 6.8 to about 7.0, from about 6.0 to about 7.0, from about 7.0 to about 8.0, from about 6.5 to about 7.5, or from about 6.5 to about 7.0. In some embodiments, the pharmaceutical composition has a pH of any of about 6.0, 6.1, 6.2, 6.3, 6.4, 6.5, 6.6, 6.7, 6.8, 6.85, 6.9, 7.0, 7.1, 7.2, 7.3, 7.4, 7.5, 7.6, 7.7, 7.8, 7.9, or 8.0. In some embodiments, the pharmaceutical composition has a pH of about 6.8±0.5. In some embodiments, the pharmaceutical composition has a pH of about 6.8 or 6.85.

### Additional Components

Acceptable carriers or excipients that are nontoxic to recipients at the dosages and concentrations employed can be employed in the pharmaceutical composition, which include antioxidants including ascorbic acid, methionine, Vitamin E, sodium metabisulfite; preservatives (such as octadecyldimethylbenzyl ammonium chloride; hexamethonium chloride; benzalkonium chloride, benzethonium chloride; phenol, butyl or benzyl alcohol: alkyl parabens such as methyl or propyl paraben; catechol; resorcinol; cyclohexanol; 3-pentanol; and m-cresol); isotonicifiers (e.g. sodium chloride); metal complexes (e.g. Zn-protein complexes); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; salt-forming counterions such as sodium; and/or chelating agents such as EDTA.

Preservatives can be added to retard microbial growth, and are typically present in a range from 0.2%-1.0/o (w/v). The addition of a preservative may, for example, facilitate the production of a multi-use (multiple-dose) formulation. Suitable preservatives for use in the present application include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

### Antioxidants

In some embodiments, the pharmaceutical composition further comprises an antioxidant, such as methionine, sodium thiosulfate, catalase, platinum, Vitamin E, sodium metabisulfite, etc. In some embodiments, the antioxidant is methionine. In some embodiments, the antioxidant is in an amount of from about 0.01 mg/ml to about 1 mg/ml, such as any of from about 0.01 mg/ml to about 0.05 mg/ml, from about 0.01 mg/ml to about 0.1 mg/ml, from about 0.1 mg/ml to about 1 mg/ml, or from about 0.05 mg/ml to about 1 mg/ml. In some embodiments, the antioxidant is in an amount of about any of 0.01 mg/ml, 0.05 mg/ml, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, or 1 mg/ml.

Antioxidants can confer stability and/or resistance to oxidation or degradation induced by free radicals to protein formulations. Oxidation or degradation due to free radicals can occur due to one or more adverse conditions, such as exposure to light, including ultraviolet, visible and/or fluorescent light, exposure to free radicals generated from other sources, and/or exposure to metal ions. Any known antioxidants can be used in the pharmaceutical compositions described herein.

Antioxidant can also be selected from the group consisting of vanillic acid, gallic acid, syringic acid, protocatechuic acid, cinnamic acid, coumaric acid, caffeic acid, ferulic acid, sinapic acid, chlorogenic acid, benzoic acid, methyl paraben, ethyl paraben, propyl paraben, butyl paraben, m-cresol, benzyl alcohol, phenoxyethanol, phenol, cytosine, thiamine, pyridoxine, fumaric acid, shikimic acid, glycolic acid, p-hydroxybenzoic acid, p-aminobenzoic acid, catechin, epicatechin, furaneol, sulisobenzone, a pharmaceutically acceptable salt or ester thereof, and mixtures thereof

### Calcium Salts

In some embodiments, the pharmaceutical composition further comprises a calcium salt. In some embodiments, the calcium salt is calcium chloride. In some embodiments, the calcium salt is in an amount of from about 0.1 mg/ml to about 10 mg/ml, such as any of from about 0.1 mg/ml to about 1 mg/ml, from about 0.5 mg/ml to about 3 mg/ml, from about 1 mg/ml to about 10 mg/ml, from about 1 mg/mi to about 5 mg/ml, or from about 5 mg/ml to about 10 mg/ml. In some embodiments, the calcium salt is in an amount of about any of 0.1 mg/ml, 0.5 mg/ml, 1 mg/ml, 2 mg/ml, 3 mg/ml, 4 mg/ml, 5 mg/ml, 6 mg/ml, 7 mg/ml, 8 mg/ml, 9 mg/ml, or 10 mg/ml. In some embodiments, the calcium salt is in an amount of about any of 1 mg/ml, 3 mg/ml, or 10 mg/ml.

Any suitable calcium salt can be used herein, including but not limited to, calcium chloride, calcium phosphate, calcium acetate, calcium citrate, calcium carbonate, etc.

### Bioactivity

As used herein, “biological activity” refers to the in vivo activities of a compound or physiological responses that result upon in vivo administration of a compound, composition or other mixture. Biological activity, thus, encompasses therapeutic effects and pharmaceutical activity of such compounds, compositions and mixtures. Biological activities can be observed in in vitro systems designed to test or use such activities. Thus, for purposes herein a biological activity of a FX polypeptide encompasses the coagulant activity.

A bioassay focuses on biological activity of a test protein (e.g., FX activator) and using it as a read out. In a bioassay, the activity of a sample is tested on a sensitive sample (e.g., human plasma) or an animal model/human of test protein-relevant disease (e.g., bleeding disorder), and the results of this activity (e.g., hemostasis) are compared to a control (e.g., other hemostatic agent). Other aspects of biological activity of FX activators include: (i) activating FX and/or promoting FXa generation; (ii) increasing thrombin generation (TG) and/or endogenous thrombin-generating potential (ETP); (iii) shortening activated partial thromboplastin time (APTT), prothrombin time (PT), and/or thrombin time (TT); (iv) promoting hemostasis, such as reducing bleeding time and/or amount; (v) reducing mortality; (vi) promoting wound healing; etc..

Various methods for determining the bioactivities of FX activators described herein, such as venom-derived FX activators (e.g., RVV-X), are known in the art, in vitro or in vivo. There are mainly two types of FX activator activity measurement techniques. One is plasma coagulation time method, which determines FX activator activity by testing the plasma coagulation time and comparing it with the coagulation time of an active reference substance. The other one is chromogenic substrate continuous rate method, which determines FX activator activity by comparing the reaction rate of the test sample vs. the active reference substance. For example, see J. Li et al. (“The activity determination of factor X activator from Vipera russelli by an enzyme rate method using chromogenic substrates,” Chinese Journal of New Drugs. 2007; 16(18); 1491-1495), and CN108089006B, the contents of each of which are incorporated herein by reference in their entirety. The general mechanism is: FX activator can convert inactive FX into hydrolytically active FXa, the generated FXa can in turn hydrolyze the synthetic chromogenic substrate Suc-Ile-Gly(yPip)Gly-Arg-pNA (SEQ ID NO: 4), and the hydrolyzed substrate can release p-nitroaniline with characteristic absorption at 405 nm. The reaction rate can be evaluated by the amount of product (p-nitroaniline) generated per unit time, and the enzyme activity is proportional to the reaction rate. The activity unit (U) of the test sample (FX activator) can then be calculated by reference to the reaction rate of an active reference. Unless otherwise specified, the activity unit (U) of FX activator (e.g., RVV-X) described herein is measured by the chromogenic substrate continuous rate method as described in CN108089006B.

The obtained product and/or intermediates after administering FX activator can be subjected to analytical testing, such as FX ELISA, TGA (thrombin generation assay), and FXa-specific activity assay. Although chromogenic substrate for the determination of FXa is very specific for the protease, but not 100%, a minor portion of cleavage of potential other targets occurs, especially at high protease concentrations. The FXa-specific activity assay can be used for measurement of FXa in presence of cross-reactive proteins. In order to determine FXa-specific activity, highly specific inhibitor for FXa (i.e., Rivaroxaban) can be used to determine the inhibitable amount, representing the protease-specific activity. Activation efficacy can be calculated and expressed as percentage of FXa generated from total FX added into reaction. TGA can be performed according to manufacturer's instructions, such as using the Technothrombin® TGA kit (Technoclone). The read-out parameter is peak thrombin concentration (PTC) in nM.

Bioactivity of FX activator (e.g., RVV-X) can also be measured by blood coagulation meter, such as for blood clotting time. Coagulation tests, such as the prothrombin time (PT; measures how well and how long it takes for blood to clot), activated partial thromboplastin time (aPTT; measures how long it takes for blood to clot), and thrombin time (TT; measures how well fibrinogen is working), can also be conducted to assess blood clotting function in subjects, reflecting bioactivitiy of FX activators. Also see Examples 1-3, 6, and 10.

### Stability

The pharmaceutical composition is preferably to be stable, in which the proteins contained within (FX activator, e.g., RVV-X) essentially retains its physical and chemical stability and integrity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Stability can be measured at a selected temperature, humidity, light, or other conditions, for a selected time period. For rapid screening, the formulation may be kept at 40° C. for 2 weeks to 1 month, at which time stability is measured. Where the formulation is to be stored at 2-8° C., generally the formulation should be stable at 30° C. or 40° C. for at least 1 month, and/or stable at 2-8° C. for at least 2 years. Where the formulation is to be stored at 30° C., generally the formulation should be stable for at least 2 years at 30° C., and/or stable at 40° C. for at least 6 months. For example, the extent of aggregation during storage can be used as an indicator of protein stability.

In some embodiments, the FX activator (e.g., RVV-X) pharmaceutical compositions described herein (e.g., RVV-X lead formulation) have excellent stability, such as physical stability, chemical stability, and/or biological stability. In some embodiments, the FX activator pharmaceutical compositions described herein have excellent thermal stability. In some embodiments, the FX activator pharmaceutical compositions described herein have superior stability under accelerated stability condition (e.g., 25° C., RH 65±5%; or 25°±2° C., RH 60%±5%), long-term storage condition (e.g., 2-8° C. for at least 3 months), and/or stress condition (e.g., high temperature (such as 40±2° C.), high humidity (such as 2-8° C., RH 92.5±5%), light (such as 2-8° C., 4500±500LX)), such as less or no fragmentation, aggregate formation, insoluble particle (particularly less insoluble particles bigger than 10 μm), and/or aggregate increment, such as compared to FX activator pharmaceutical compositions using HSA as stabilizer.

Stability of protein, in particular the susceptibility to aggregation, is primarily determined by the conformational and the colloidal stability of the protein molecules. It is generally believed that the first step in non-native protein aggregation, which is the most prevalent form of aggregation, is a slight perturbation of the molecular structure, e.g., a partial unfolding of the protein, i.e., a conformational change. This is determined by the conformational stability of the protein. In the second step, the partially unfolded molecules then come into close proximity, being driven by diffusion and random Brownian motion, to form aggregates. This second step is primarily governed by the colloidal stability of the molecules (see Chi et al., Roles of conformational stability and colloidal stability in the aggregation of recombinant human granulocyte colony stimulating factor. Protein Science, 2003 May; 12(5). 903-913). As used herein, the term “stability” generally is related to maintaining the integrity or to minimizing the degradation, denaturation, aggregation, or unfolding of a biologically active agent such as a protein. As used herein, “improved stability” generally means that, under conditions known to result in degradation, denaturation, aggregation or unfolding, the protein of interest (or protein in pharmaceutical composition of interest) maintains greater stability compared to a control protein (or same protein in a control pharmaceutical composition).

Differential scanning calorimetry (DSC) and differential scanning fluorimetry (DSF) are well known techniques in the art that are used to predict the stability of a protein formulation. Specifically, these techniques can be used to determine the unfolding temperature (Tm) of a protein in given formulation. It is standard in the art to correlate high Tm measurements for a protein in given formulation with more robust and stable protein formulations for long-term, shelf-stable storage.

A “stable” protein (or formulation), e.g., an FX activator (e.g., RVV-X) pharmaceutical composition described herein, essentially retains its physical stability and/or chemical stability and/or biological activity during the manufacturing process, upon storage, and/or upon reconstitution. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. (1993) Adv. Drug Delivery Rev. 10: 29-90. For example, in one embodiment, the stability of the protein (or formulation) is determined according to the percentage of monomer protein in the solution, with a low percentage of degraded (e.g., fragmented) and/or aggregated protein. Preferably, a protein (or formulation) is stable at room temperature or at 40° C. for at least 1 month and/or stable at about 2-8° C. for at least 6 months, or for at least 1 year or for at least 2 years. Furthermore, the protein (or formulation) is preferably stable following freezing (to, e.g., −70° C. to −80° C.) and thawing, hereinafter referred to as a “freeze/thaw cycle.”

A protein (or formulation), e.g., an FX activator (e.g., RVV-X) pharmaceutical composition described herein, “retains its physical stability” if it shows substantially no signs of instability, e.g., aggregation, precipitation and/or denaturation, upon visual examination of appearance (e.g., color, fullness, smoothness, clarity, wall-attachment), or as measured by UV light scattering, size exclusion chromatography with multi-angle light scattering (SEC-MALS), SEC, SEC-HPLC, reversed-phase chromatography (RP-HPLC), or imaged capillary isoelectric focusing (iCIEF). Aggregation is a process whereby individual protein molecules or complexes associate covalently or non-covalently to form aggregates. Aggregation can proceed to the extent that a visible precipitate is formed.

A protein (or formulation), e.g., an FX activator (e.g., RVV-X) pharmaceutical composition described herein, “retains its chemical stability” in a formulation, if the chemical stability at a given time is such that the protein is considered to still retain its biological activity (e.g., as mentioned in “Bioactivity” subsection above). Chemical stability can be assessed by, e.g., detecting and quantifying chemically altered forms of the protein. Chemical alteration may involve size modification (e.g. clipping) which can be evaluated using size exclusion chromatography (SEC), SDS-PAGE (e.g., reduced or non-reduced), capillary electrophoresis sodium dodecyl sulfate (CE-SDS, reduced or non-reduced), and/or matrix-assisted laser desorption ionization/time-of-flight mass spectrometry (MALDI/TOF MS), for example. Other types of chemical alteration include charge alteration (e.g. occurring as a result of deamidation or oxidation) which can be evaluated by ion-exchange chromatography, for example.

A protein (or formulation), e.g., an FX activator (e.g., RVV-X) pharmaceutical composition described herein, “retains its biological activity” in a formulation, if the protein, in a pharmaceutical formulation is biologically active for its intended purpose. For example, biological activity is retained if the biological activity of the protein, in the formulation is changed (e.g., increased or decreased) no more than about 30%, about 20/6, or about 10% (within the errors of the assay) of the biological activity exhibited at the time the formulation was prepared.

One of skill in the art will appreciate that stability of a protein (e.g., FX activator such as RVV-X) is dependent on other features in addition to the composition of the formulation. For example, stability can be affected by temperature, pressure, humidity, pH, light, and external forms of radiation. Stability of a protein in a protein formulation can be determined by various means. In some embodiments, the protein stability is determined by SEC. SEC separates analytes (e.g., macromolecules such as proteins) on the basis of a combination of their hydrodynamic size, diffusion coefficient, and surface properties. Thus, for example, SEC can separate FX activators (e.g., RVV-X) described herein in their natural three-dimensional conformation from proteins in various states of denaturation, and/or proteins that have been degraded. In SEC, the stationary phase is generally composed of inert particles packed into a dense three-dimensional matrix within a glass or steel column. The mobile phase can be pure water, an aqueous buffer, an organic solvent, mixtures of these, or other solvents. The stationary-phase particles have small pores and/or channels which will only allow species below a certain size to enter. Large particles are therefore excluded from these pores and channels, but the smaller particles are removed from the flowing mobile phase. The time particles spend immobilized in the stationary-phase pores depends, in part, on how far into the pores they can penetrate. Their removal from the mobile phase flow causes them to take longer to elute from the column and results in a separation between the particles based on differences in their size.

In some embodiments, SEC is combined with an identification technique to identify or characterize proteins (e.g., FX activator such as RVV-X), or fragments thereof. Protein identification and characterization can be accomplished by various techniques, including but not limited chromatographic techniques, e.g., high-performance liquid chromatography (HPLC), Capillary Electrophoresis-Sodium Dodecyl Sulfate (CE-SDS), immunoassays, electrophoresis, ultra-violet/visible/infrared spectroscopy, raman spectroscopy, surface enhanced raman spectroscopy, mass spectroscopy, gas chromatography, static light scattering (SLS), Fourier Transform Infrared Spectroscopy (FTIR), circular dichroism (CD), urea-induced protein unfolding techniques, intrinsic tryptophan fluorescence, differential scanning calorimetry, and/or ANS protein binding.

In some embodiments, sample formulations (e.g., FX activator pharmaceutical compositions described herein, e.g., using sucrose as stabilizer) and reference formulations (e.g., FX activator pharmaceutical composition comprising HSA as stabilizer, or standards) are optionally assayed prior to a treatment phase to determine the content of monomer, aggregated, insoluble particles, and/or fragmented protein (and/or fragmentation increase %, aggregation increase %, etc.), such as described below in the Example 6. Subsequently, each of the protein formulations undergoes a treatment phase. For example, each protein formulation may be stored for an extended period (e.g., 3 months, 6 months, 12 months, or longer) at a specific temperature (e.g., 40° C., 25° C., or 5° C.). In some embodiments, the protein formulations undergo a physical stress test such as stir-stress assay. In some embodiments, the protein formulations undergo a reconstitution test or solvent compatibility test. In some embodiments, the protein formulations undergo accelerated stability test, such as treated under accelerated stress, including high temperature (e.g., 40° C. or above), high humidity, light, and/or low pH, etc. In some embodiments, the protein formulations undergo cycles of freezing and thawing. In some embodiments, samples of the same protein formulation receive differential treatment, e.g., storage for a period of time in different temperatures/humidity/light. Following the treatment phase, the protein formulations are assayed to determine the content of protein monomer, aggregates, insoluble particles, and/or fragments (and/or fragmentation increase %, aggregation increase %, etc.). The protein formulations can also be assayed for physical appearance or pH change.

Stability, such as physical stability of a composition or formulation, may be assessed by methods well-known in the art, including measurement of a sample's apparent attenuation of light (absorbance, or optical density). Such a measurement of light attenuation relates to the turbidity of a formulation. The turbidity of a formulation is partially an intrinsic property of the protein dissolved in solution and is commonly determined by nephelometry, and measured in Nephelometric Turbidity Units (NTU).

The degree of turbidity, e.g., as a function of the concentration of one or more of the components in the solution, e.g., protein and/or salt concentration, is also referred to as the “opalescence” or “opalescent appearance” of a formulation. The degree of turbidity can be calculated by reference to a standard curve generated using suspensions of known turbidity. Reference standards for determining the degree of turbidity for pharmaceutical compositions can be based on the European Pharmacopeia criteria (European Pharmacopoeia, Fourth Ed., Directorate for the Quality of Medicine of the Council of Europe (EDQM), Strasbourg, France). According to the European Pharmacopeia criteria, a clear solution is defined as one with a turbidity less than or equal to a reference suspension which has a turbidity of approximately 3 according to European Pharmacopeia standards. Nephelometric turbidity measurements can detect Rayleigh scatter, which typically changes linearly with concentration, in the absence of association or nonideality effects. Other methods for assessing physical stability of a pharmaceutical protein are well-known in the art, e.g., SEC or analytical ultracentrifucation.

In some embodiments, stability refers to formulation containing an FX activator (e.g., RVV-X) described herein having low to undetectable levels of particle formation, or low to undetectable levels of formation of particles bigger than 10 μm in diameter. The phrase “low to undetectable levels of particle formation” (e.g., insoluble particles) as used herein refers to samples containing less than about any of 2500 particles/mL, 2000 particles/mL, 1800 particles/mL, 1500 particles/mL, 1400 particles/mL, 1200 particles/mL, 1000 particles/mL, 950 particles/mL, 900 particles/mL, 700 particles/mL, 500 particles/mL, 400 particles/mL, 300 particles/mL, 200 particles/mL, 150 particles/mL, 120 particles/mL, 110 particles/mL, 100 particles/mL, 50 particles/mL, 30 particles/mL, 20 particles/ml, 15 particles/ml, 10 particles/ml, 5 particles/ml, or less, as determined by HIAC analysis, visual analysis, or according to “Chinese Pharmacopoeia,” 2015 Edition, IV General Principles. In some embodiments, the FX activator pharmaceutical compositions described herein (e.g., after reconstitution) have no more than about 350 insoluble particles/mL, such as no more than about 200 or no more than about 150 insoluble particles/mL. In some embodiments, the FX activator pharmaceutical compositions described herein (e.g., after reconstitution) have no more than about 2500 insoluble particles/mL, such as no more than about 1500 or no more than about 1000 insoluble particles/mL, after about 6 months of accelerated storage condition.

“Substantial protein aggregation” refers to a level of protein aggregation in a protein formulation that is substantially greater than the level of protein aggregation in a reference protein formulation. The reference protein formulation may be the same protein formulation before a period of storage or before a treatment (e.g., before subjected to a destabilizing condition, such as elevated temperature, humidity, pH, and/or to long term storage.). The reference protein formulation may be a different protein formulation (e.g., other FX activator formulation using HSA as stabilizer) tested under the same condition.

“Substantially free of protein aggregation” refers to proteins (or formulations) of the invention that do not have a significantly greater level or percentage of aggregated protein than a reference formulation. For example, this phrase refers to proteins (or formulations) in which the level of protein aggregation is less than about 15%, 14%, 13%, 12%, 11%, 10%, 9%, 8%, 7%, 6%, 5%, 4%, 3%, 2%, 1%, 0.5%, 0.2% or 0.1%. The level of protein aggregation may be determined using standard techniques known in the art. In some embodiments, the FX activator (e.g., RVV-X) pharmaceutical composition described herein is substantially free of protein aggregation (e.g., under accelerated stability test or long-term storage). In some embodiments, the FX activator (e.g., RVV-X) pharmaceutical composition (e.g., after reconstitution) has at most about 15% protein aggregation, such as at most about any of 14%, 13%, 12%, 11%, 10%, 9%, 8%, 7%, 6%, 5%, 4%, 3%, 2%, 1%, or 0.5% protein aggregation (e.g., under accelerated stability test, repeated (e.g., up to 5) freeze/thaw, or long-term storage). In some embodiments, the FX activator (e.g., RVV-X) pharmaceutical composition described herein (e.g., after reconstitution) has no protein aggregation (e.g., under accelerated stability test, repeated (e.g., up to 5) freeze/thaw, or long-term storage). In some embodiments, the FX activator (e.g., RVV-X) pharmaceutical composition has no more than about 15% of aggregation increase, such as no more than any of about 14%, 13%, 12%, 11%, 10%, 9%, 8%, 7%, 6%, 5%, 4%, 3% 2%, 1%, or 0.5% aggregation increase (e.g., under accelerated stability test, repeated (e.g., up to 5) freeze/thaw, or long-term storage). In some embodiments, the stability is measured by SEC or SEC-HPLC. In some embodiments, the stability is measured by CE-SDS.

In some embodiments, stability refers to reduced protein fragmentation. The term “low to undetectable levels of fragmentation” as used herein refers to samples containing equal to or more than 80%, 85%, 90%, 95%, 98% or 99% of the total protein, for example, in a single peak as determined by HPSEC, or in multiple peaks (e.g., as many peaks as there are subunits) by reduced Capillary Gel Electrophoresis (rCGE), representing the non-degraded protein or a non-degraded fragment thereof, and containing no other single peaks having more than 5%, more than 4%, more than 3%, more than 2%, more than 1%, or more than 0.5% of the total protein in each. The term “reduced Capillary Gel Electrophoresis” as used herein refers to capillary gel electrophoresis under reducing conditions sufficient to reduce disulfide bonds in a test protein. In some embodiments, the FX activator (e.g., RVV-X) pharmaceutical composition (e.g., after reconstitution) has about 0,% to about 15% fragmentation, such as about 0% to about 12% fragmentation (e.g., under accelerated stability test or long-term storage). In some embodiments, the FX activator (e.g., RVV-X) pharmaceutical composition (e.g., after reconstitution) has no more than about 30% of fragmentation, such as no more than any of about 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% fragmentation (e.g., under accelerated stability test or long-term storage). In some embodiments, the FX activator (e.g., RVV-X) pharmaceutical composition (e.g., after reconstitution) has no fragmentation (e.g., under accelerated stability test or long-term storage). In some embodiments, the FX activator (e.g., RVV-X) pharmaceutical composition (e.g., after reconstitution) has no more than about 30% of fragmentation increase, such as no more than any of about 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% fragmentation increase (e.g., under accelerated stability test or long-term storage). In some embodiments, the FX activator (e.g., RVV-X) pharmaceutical composition (e.g., after reconstitution) has at least about 75% main peak, such as at least about any of 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% main peak (e.g., under accelerated stability test or long-term storage). In some embodiments, the stability is measured by SEC. In some embodiments, the stability is measured by CE-SDS.

In some embodiments, “stability” or “stable” characteristics of the pharmaceutical composition include one or more of appearance, moisture content, reconstitution time, pH value, and biological activity.

In some embodiments, the FX activator (e.g., RVV-X) pharmaceutical composition after lyophilization has a moisture content of at most about 5%, such as at most about any of 4.5%, 4.0%, 3.5%, 3.0%, 2.9%, 2.8%, 2.7%, 2.6%, 2.5%, 2.4%, 2.3%, 2.2%, 2.1%, 2.0%, 1.9%, 1.8%, 1.7%, 1.6%, 1.5%, 1.4%, 1.3%, 1.2%, 1.1%, 1.0%, 0.8%, 0.5%, or less. In some embodiments, the FX activator (e.g., RVV-X) pharmaceutical composition after lyophilization has a reconstitution time of at most about 30 seconds, such as at most about any of 28, 26, 24, 22, 20, 18, 16, 15, 14, 13, 12, 11, 10, 9, 8, 7, 6, 5, 4, 3 seconds, or less. In some embodiments, the FX activator (e.g., RVV-X) pharmaceutical composition after lyophilization has a shrinkage of at most about 10% (e.g., compared to exquisite/full powder), such as at most about any of 9%, 8%, 7%, 6%, 5%, 4%, 3%, 2% 1%, or less. In some embodiments, the lyophilized FX activator (e.g., RVV-X) pharmaceutical composition after reconstitution dissolves into colorless and clear liquid. In some embodiments, the lyophilized FX activator (e.g., RVV-X) pharmaceutical composition (e.g., after reconstitution) varies (e.g., increases or decreases, such as compared to before lyophilization, or before the influenced storage condition starts) in pH for at most about 1, such as at most about any of 0.9, 0.8, 0.7, 0.6, 0.5, 0.4, 0.3, 0.2, 0.1, or less (such as 0). In some embodiments, the lyophilized FX activator (e.g., RVV-X) pharmaceutical composition (e.g., after reconstitution) varies (e.g., increases or decreases, such as compared to before lyophilization, or before the influenced storage condition starts) in biological activity for at most about 30%, such as at most about any of 25%, 20%, 19%, 18%, 17%, 16%, 15%, 14%, 13%, 12%, 11%, 10%, 9%, 8%, 7%, 6%, 5%, 4%, 3%, 2% 1%, 0.9%, 0.8%, 0.7%., 0.6%, 0.5%, 0.4%, 0.3%, 0.2%, 0.1%, or less (such as 0%). In some embodiments, the above characteristics apply for standard storage condition (e.g., 2-8° C. at least about 2 hours after reconstitution), high temperature condition (e.g., at least about any of 35° C., 36° C., 38° C., 40° C., 42° C., 45° C., 50′C, 55° C., 60° C., 65° C., 70° C., or higher), high humidity condition (e.g., 2-8° C., relative humidity (RH) of at least about any of 60%, 65%, 70%, 75%, 80%, 85%, 87.5%, 90%, 92.5%, 97.5%, or higher), light condition (e.g., 2-8° C., at least about any of 4000 lx, 4200 lx, 4400 lx, 4500 lx, 4600 lx, 4800 lx, 5000 lx, 5500 lx, or higher), long-term condition (e.g., 2-8° C. or below −70° C. for at least about 1, 2, 3, 4, 5, 6, 7, 8, 9, 10, 11, 12, 18, 24, 30, 36 months, or longer), freeze-thaw condition (e.g., −70° C./2~8° C. for 1, 2, 3, 4, 5, or more times), or accelerated storage condition (e.g., 25° C.+2° C., RH of at least about any of 60%, 65%, 70%, 75%, 80%, 85%, 87.5%, 90%, 92.5%, 97.5%, or higher), such as for at least about 2 hrs, 4 hrs, 6 hrs, 8 hrs, 10 hrs, 12 hrs, 16 hrs, 18 hrs, 24 hrs, 2 days, 3 days, 4 days, 5 days, 6 days, 7 days, 10 days, 12 days, 14 days, 16 days, 20 days, 25 days, 30 days, 1 month, 2 months, 3 months, 6 months, 30 months, or longer under corresponding storage condition. In some embodiments, the FX activator (e.g., RVV-X) pharmaceutical composition (e.g., reconstituted) decreases in biological activity for at most about 20% (such as at most about any of 15%, 10%, 9%, 8%, 7%, 6%, 5%, 4%, 3%, 2% 1%, 0.9%, 0.8%, 0.7%, 0.6%, 0.5%, 0.4%, 0.3%, 0.2%, 0.1%, or less (such as 0%)) compared to before the influenced storage condition (e.g., high temperate, high humidity, light, long-term, or acceleration) starts, such as within at most about 30 months (e.g., at most about any of 18 months, 12 months, 10 months, 9 months, 8 months, 7 months, 6 months, 5 months, 4 months, 3 months, 2 months, 1 month, 28 days, 20 days, 14 days, 10 days, 7 days, 5 days, 3 days, 1 day) under the influenced storage condition.

In some embodiments, the FX activator (e.g., RVV-X) pharmaceutical composition described herein may comprise less than about 10% (preferably less than about 5%) of the FX activator present as an aggregate or an insoluble particle in the pharmaceutical composition (e.g., after reconstitution from lyophilized powder). In some embodiments, the pharmaceutical composition is lyophilized. In some embodiments, the pharmaceutical composition (e.g., lyophilized) is stable at 25° C. for at least about 4 hours (such as at least about any of 6, 8, 10, 12, 14, 16, 18, 20, 22, 24, 36, 48, 60, 72 hours, or longer). In some embodiments, the pharmaceutical composition (e.g., lyophilized) is stable at 2-8° C. (e.g., any of 2, 3, 4, 5, 6, 7, or 8° C.) for at least about 3 months (such as at least about any of 4, 6, 8, 10, 12, 14, 16, 18, 20, 22, 24, 36, 48, 60 months, or longer). In some embodiments, the pharmaceutical composition (e.g., lyophilized) is stable at 25° C. under accelerated stability condition for at least about 3 months (such as at least about any of 4, 6, 8, 10, 12, 14, 16, 18, 20, 22, 24, 36, 48, 60 months, or longer). In some embodiments, the pharmaceutical composition comprises less than about 100 (e.g., less than about any of 90, 80, 70, 60, 50, 40, 30, 20, or 10) of insoluble particles with a diameter of more than 10 μm (e.g., at least about any of 10, 15, 20, 25, 30 μm or more) after storage at 25° C. under accelerated stability condition for at least about 6 months (e.g., at least about any of 8, 10, 12, 14, 16, 18, 20, 22, 24, 36, 48 months, or longer). In some embodiments, the lyophilized pharmaceutical composition is stable at 2-8° C. (e.g., any of 2, 3, 4, 5, 6, 7, or 8° C.) for at least about 6 months (such as at least about any of 9, 10, 12, 14, 16, 18, 20, 22, 24, 36, 48, 60 months, or longer), such as at least about 30 months. In some embodiments, the lyophilized pharmaceutical composition is stable at 25° C.+2° C. (e.g., any of 23, 24, 25, 26, or 27° C.) for at least about 1 months (such as at least about any of 2, 3, 4, 5, 6, 10, 12 months, or longer), such as at least about 6 months. For example, the biological activity of FX activator (e.g., RVV-X) varies (e.g., increases or decreases, such as compared to before the long term or accelerated storage condition starts) for at most about 10% (such as at most about any of 9%, 8%, 7%, 6%, 5%, 4%, 3%, 2% 1%, 0.9%, 0.8%, 0.7%, 0.6%, 0.5%, 0.4%, 0.3%, 0.2%, 0.1%, or less (such as 0%); pH varies (e.g., increases or decreases, such as compared to before the long term or accelerated storage condition starts) for at most about 0.5 (such as at most about any of 0.45, 0.4, 0.35, 0.3, 0.25, 0.2, 0.15, 0.1, 0.09, 0.08, 0.05, 0.02, 0.01, or less (such as 0)); or moisture content varies (e.g., increases or decreases, such as compared to before the long term or accelerated storage condition starts) for at most about 5% (such as at most about any of 4%, 3%, 2% 1%, 0.9%, 0.8%, 0.7%, 0.6%, 0.5%, 0.4%, 0.3%, 0.2%, 0.1%, or less (such as 0%)).

In some embodiments, the FX activator pharmaceutical composition is not lyophilized. In some embodiments, the pharmaceutical composition (e.g., non-lyophilized) is stable at below −70° C. for at least about 6 months (such as at least about any of 8, 10, 12, 14, 16, 18, 20, 22, 24, 36, 48, 60 months, or longer), such as at least about 24 months. In some embodiments, the pharmaceutical composition (e.g., non-lyophilized) is stable at 2-8° C. for at least about 1 months (such as at least about any of 2, 3, 4, 5, 6, 8, 10, 12 months, or longer), such as at least about 6 months. In some embodiments, the pharmaceutical composition (e.g., non-lyophilized) is stable after freeze/thaw condition (e.g., frozen at −70° C. for 24 hours, and thawed at 2-8° C.) for at least once (such as at least about any of twice, 3, 4, 5, 6, 10 times, or more), such as up to about 5 times. For example, the protein content varies (e.g., increases or decreases, such as compared to before the long term, accelerated, or freeze/thaw storage condition starts) for at most about 10% (such as at most about any of 9%, 8%, 7%, 6%, 5%, 4%, 3%, 2% 1%, 0.9%, 0.8%, 0.7%, 0.6%, 0.5%, 0.4%, 0.3%, 0.2%, 0.1%, or less (such as 0%)); the biological activity of FX activator (e.g., RVV-X) varies (e.g., increases or decreases, such as compared to before the long term, accelerated, or freeze/thaw storage condition starts) for at most about 10% (such as at most about any of 9%, 8%, 7%, 6%, 5%, 4%, 3%, 2% 1%, 0.9%, 0.8%, 0.7%, 0.6%, 0.5%, 0.4%, 0.3%, 0.2%, 0.1%, or less (such as 0%); pH varies (e.g., increases or decreases, such as compared to before the long term, accelerated, or freeze/thaw storage condition starts) for at most about 0.5 (such as at most about any of 0.45, 0.4, 0.35, 0.3, 0.25, 0.2, 0.15, 0.1, 0.09, 0.08, 0.05, 0.02, 0.01, or less (such as 0)); or purity (e.g., measured by SEC-HPLC) varies (e.g., increases or decreases, such as compared to before the long term, accelerated, or freeze/thaw storage condition starts) for at most about 10% (such as at most about any of 9%, 8%, 7%, 6%, 5%, 4%, 3%, 2% 1%, 0.9%, 0.8%, 0.7%, 0.6%, 0.5%, 0.4%, 0.3%, 0.2%, 0.1%, or less (such as 0%)).

FX activator (e.g., RVV-X) pharmaceutical compositions described herein can be reconstituted with any suitable solvent, such as for intravenous administration. In some embodiments, the solvent is sterilized water for injection, 0.9% sodium chloride injection, or 5% glucose injection. In some embodiments, the solvent is 0.9% sodium chloride injection. In some embodiments, the FX activator (e.g., RVV-X) pharmaceutical composition varies (e.g., increases or decreases) in biological activity for at most about 30%, such as at most about any of 25%, 20%, 15%, 10%, 9%, 8%, 7%, 6%, 5%, 4%, 3%, 2% 1%, 0.9%, 0.8%, 0.7%, 0.6%, 0.5%, 0.4%, 0.3%, 0.2%, 0.1%, or less (such as 0%), after reconstitution and storing at room temperate for at least about 2 hours (e.g., at least about any of 3, 4, 5, 6, 7, 8, or longer), such as for about 8 hours, compared to 0 hour after reconstitution. In some embodiments, the FX activator (e.g., RVV-X) pharmaceutical composition varies (e.g., increases or decreases) in pH for at most about 0.5, such as at most about any of 0.45, 0.4, 0.35, 0.3, 0.25, 0.2, 0.15, 0.1, 0.09, 0.08, 0.05, 0.02, 0.01, or less (such as 0), after reconstitution and storing at room temperate for at least about 2 hours (e.g., at least about any of 3, 4, 5, 6, 7, 8, or longer), such as for about 8 hours, compared to 0 hour after reconstitution. In some embodiments, the FX activator (e.g., RVV-X) pharmaceutical composition varies (e.g., increases or decreases) in osmotic pressure for at most about 5%, such as at most about any of 4%, 3%, 2% 1%, 0.9%, 0.8%, 0.7%, 0.6%, 0.5%, 0.4%, 0.3%, 0.2%, 0.1%, or less (such as 0%), after reconstitution and storing at room temperate for at least about 2 hours (e.g., at least about any of 3, 4, 5, 6, 7, 8, or longer), such as for about 8 hours, compared to 0 hour after reconstitution. In some embodiments, the FX activator (e.g., RVV-X) pharmaceutical composition (e.g., after reconstitution) has an osmotic pressure from about 80 mOsm/kg to about 450 mOsm/kg, such as any of from about 100 mOsm/kg to about 400 mOsm/kg, from about 100 mOsm/kg to about 150 mOsm/kg, from about 150 mOsm/kg to about 400 mOsm/kg, from about 350 mOsm/kg to about 400 mOsm/kg, from about 100 mOsm/kg to about 150 mOsm/kg, from about 200 mOsm/kg to about 400 mOsm/kg, from about 240 mOsm/kg to about 400 mOsm/kg, from about 270 mOsm/kg to about 370 mOsm/kg, from about 300 mOsm/kg to about 400 mOsm/kg, from about 250 mOsm/kg to about 350 mOsm/kg, from about 350 mOsm/kg to about 400 mOsm/kg, from about 300 mOsm/kg to about 330 mOsm/kg, or from about 280 mOsm/kg to about 320 mOsm/kg. Normal osmotic pressure of human plasma is from about 280 mOsm/kg to about 320 mOsm/kg. In some embodiments, the FX activator (e.g., RVV-X) pharmaceutical composition (e.g., after reconstitution) is isotonic or substantially isosmotic, or within about 50% (e.g., 40%, 30%, 20%, 10%, 5%, or lower) variation of the range of from about 280 mOsm/kg to about 320 mOsm/kg. In some embodiments, the FX activator (e.g., RVV-X) pharmaceutical composition remains colorless clear liquid after reconstitution and storing at room temperate for at least about 2 hours (e.g., at least about any of 3, 4, 5, 6, 7, 8, or longer), such as for about 8 hours.

In some embodiments, the FX activator (e.g., RVV-X) pharmaceutical composition (e.g., lyophilized) has a shelf life of at least about 15 days, such as at least about any of 20 days, 1 month, 2 months, 3 months, 6 months, 1 year, 2 years, 3 years, or longer, for example, at about 2-25° C., such as about 2-8° C. As used herein, “shelf life” means that the storage period during which an active ingredient such as a therapeutic protein (e.g., FX activator described herein) in a pharmaceutical formulation has minimal degradation (e.g., not more than about 5% degradation, such as not more than about 4%, 3%, or 2% degradation) when the pharmaceutical formulation is stored under specified storage conditions, for example, 2-8° C. Exemplary techniques for assessing protein or formulation stability include SEC-HPLC to detect, e.g., aggregation, reverse phase (RP)-HPLC to detect, e.g. protein fragmentation, ion exchange-HPLC to detect, e.g., changes in the charge of the protein, mass spectrometry, fluorescence spectroscopy, circular dichroism (CD) spectroscopy, Fourier transform infrared spectroscopy (FT-IR), and Raman spectroscopy to detect protein conformational changes. All of these techniques can be used singly or in combination to assess the degradation of the protein in the pharmaceutical formulation and determine the shelf life of that formulation. In some embodiments, the pharmaceutical formulations of the present invention exhibit degradation (e.g., fragmentation, aggregation, or unfolding) of not more than about 5% (e.g., not more than about 4%, 3%, 2%, or 1%) for at least about 15 days (e.g., at least about any of 20 days, 1 month, 2 months, 3 months, 6 months, 1 year, 2 years, 3 years, or longer) when stored at about 2-8° C.

### H. Methods of Preparing the FX Activator Pharmaceutical Composition

The present invention also provides methods of preparing any of the FX activator (e.g., RVV-X) pharmaceutical compositions described herein (e.g., RVV-X lead formulation). In some embodiments, there is provided a method of preparing an FX activator (e.g., RVV-X) pharmaceutical composition, comprising formulating an FX activator (e.g., RVV-X) with any of the pharmaceutical composition components described herein. In some embodiments, the method further comprises lyophilizing the pharmaceutical composition. In some embodiments, the method further comprises reconstituting the lyophilized pharmaceutical composition. In some embodiments, a stock solution of the pharmaceutical composition is prepared, then diluted as needed. Also see Examples 5, 7, and 8.

In some embodiments, there is provided a method of preparing an FX activator (e.g., RVV-X) pharmaceutical composition (e.g., RVV-X lead formulation), comprising formulating an FX activator (e.g., RVV-X) in an amount of from about 0.1 U/mL to about 200 U/mL (e.g., about 10 U/mL) with i) a stabilizer (e.g., sucrose) in an amount of from about 2 mg/ml to about 100 mg/ml (e.g., about 30 mg/ml); ii) a buffering agent (e.g., histidine) in an amount of from about 0.1 mg/ml to about 50 mg/ml (e.g., about 3 mg/ml); iii) a surfactant (e.g., polysorbate 20) in an amount of from about 0.001% (w/v) to about 0.1% (w/v) (e.g., about 0.02% (w/v)); and iv) a tonicity agent (e.g., mannitol) in an amount of from about 1 mg/ml to about 100 mg/ml (e.g., about 40 mg/ml); wherein the pharmaceutical composition has a pH of from about 6.0 to about 8.0 (e.g., about 6.85). In some embodiments, the FX activator is RVV-X. In some embodiments, the RVV-X is isolated from Daboia russellii siamensis venom. In some embodiments, the method further comprises lyophilizing the pharmaceutical composition.

In some embodiments, there is provided a method of preparing an FX activator pharmaceutical composition (e.g., RVV-X lead formulation), comprising formulating RVV-X in an amount of about 10 U/mL with i) sucrose in an amount of about 30 mg/ml; ii) histidine in an amount of about 3 mg/ml; iii) polysorbate 20 in an amount of about 0.02% (w/v); and iv) mannitol in an amount of about 40 mg/ml; wherein the pharmaceutical composition has a pH of about 6.85. In some embodiments, the RVV-X is isolated from Daboia russellii siamensis venom. In some embodiments, the method further comprises lyophilizing the pharmaceutical composition.

The present invention also provides isolated nucleic acids encoding any of the FX activators (e.g., RVV-X) described herein, and vectors comprising such nucleic acids. Also provided are isolated host cells (e.g., E. coli, CHO cells, HEK 293 cells, Hela cells, or COS cells) comprising nucleic acids or vectors encoding any of the FX activators (e.g., RVV-X) described herein. In some embodiments, the isolated nucleic acid further encodes a signal peptide sequence at the N-terminus of the FX activator (e.g., N-terminus of each polypeptide chain of the RVV-X).

In some embodiments, the vector comprising a nucleic acid encoding any of the FX activators (e.g., RVV-X) described herein is suitable for replication and integration in eukaryotic cells, such as mammalian cells (e.g., CHO cells, HEK 293 cells, Hela cells, COS cells). In some embodiments, the vector is a viral vector. In some embodiments, the vector is a non-viral vector.

A number of viral based systems have been developed for gene transfer into mammalian cells. Examples of viral vectors include, but are not limited to, adenoviral vectors, adeno-associated virus vectors, lentiviral vector, retroviral vectors, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals. Retroviruses provide a convenient platform for gene delivery systems. The heterologous nucleic acid can be inserted into a vector and packaged in retroviral particles using techniques known in the art. The recombinant virus can then be isolated and delivered to the engineered mammalian cell in vitro or ex vivo. A number of retroviral systems are known in the art. In some embodiments, adenovirus vectors are used. A number of adenovirus vectors are known in the art. In some embodiments, lentivirus vectors are used. In some embodiments, self-inactivating lentiviral vectors are used. For example, self-inactivating lentiviral vectors carrying the construct protein coding sequence(s) can be packaged with protocols known in the art. The resulting lentiviral vectors can be used to transduce a mammalian cell using methods known in the art. Vectors derived from retroviruses such as lentivirus are suitable tools to achieve long-term gene transfer, because they allow long-term, stable integration of a transgene and its propagation in progeny cells. Lentiviral vectors also have low immunogenicity, and can transduce non-proliferating cells.

In some embodiments, the vector is a non-viral vector. In some embodiments, the vector is a transposon, such as a Sleeping Beauty (SB) transposon system, or a PiggyBac transposon system. In some embodiments, the vector is a polymer-based non-viral vector, including for example, poly (lactic-co-glycolic acid) (PLGA) and poly lactic acid (PLA), poly (ethylene imine) (PEI), and dendrimers. In some embodiments, the vector is a cationic-lipid based non-viral vector, such as cationic liposome, lipid nanoemulsion, and solid lipid nanoparticle (SLN). In some embodiments, the vector is a peptide-based gene non-viral vector, such as Poly-L-lysine. Any of the known non-viral vectors suitable for genome editing can be used for introducing the protein-encoding nucleic acid(s) to the host cells. See, for example, Yin H. et al., Nature Rev. Genetics (2014) 15:521-555; Aronovich E L et al. “The Sleeping Beauty transposon system: a non-viral vector for gene therapy.” Hum. Mol. Genet. (2011) R1: R14-20; and Zhao S. et al. “PiggyBac transposon vectors: the tools of the human gene editing.” Transl. Lung Cancer Res. (2016) 5(1): 120-125, which are incorporated herein by reference. In some embodiments, any one or more of the nucleic acids or vectors encoding the FX activators (e.g., RVV-X) described herein is introduced to the host cells (e.g., CHO, HEK 293, Hela, or COS) by a physical method, including, but not limited to electroporation, sonoporation, photoporation, magnetofection, hydroporation.

In some embodiments, the vector contains a selectable marker gene or a reporter gene to select cells expressing the FX activators (e.g., RVV-X) described herein from the population of host cells transfected through vectors (e.g., lentiviral vectors, pTT5 vectors). Both selectable markers and reporter genes may be flanked by appropriate regulatory sequences to enable expression in the host cells. For example, the vector may contain transcription and translation terminators, initiation sequences, and promoters useful for regulation of the expression of the nucleic acid sequences.

The nucleic acid can be cloned into the vector using any known molecular cloning methods in the art, including, for example, using restriction endonuclease sites and one or more selectable markers. In some embodiments, the nucleic acid is operably linked to a promoter. Varieties of promoters have been explored for gene expression in prokaryotic cells or eukaryotic cells (e.g., mammalian cells), and any of the promoters known in the art may be used in the present invention. Promoters may be roughly categorized as constitutive promoters or regulated promoters, such as inducible promoters.

In some embodiments, the nucleic acid encoding the FX activators (e.g., RVV-X) described herein is operably linked to a constitutive promoter. Constitutive promoters allow heterologous genes (also referred to as transgenes) to be expressed constitutively in the host cells. Exemplary promoters contemplated herein include, but are not limited to, cytomegalovirus immediate-early promoter (CMV), human elongation factors-1alpha (hEF1α), ubiquitin C promoter (UbiC), phosphoglycerokinase promoter (PGK), simian virus 40 early promoter (SV40), chicken β-Actin promoter coupled with CMV early enhancer (CAGG), a Rous Sarcoma Virus (RSV) promoter, a polyoma enhancer/herpes simplex thymidine kinase (MC1) promoter, a beta actin (β-ACT) promoter, a “myeloproliferative sarcoma virus enhancer, negative control region deleted, d1587rev primer-binding site substituted (MND)” promoter. The efficiencies of such constitutive promoters on driving transgene expression have been widely compared in a huge number of studies.

In some embodiments, the nucleic acid encoding the FX activators (e.g., RVV-X) described herein is operably linked to an inducible promoter. Inducible promoters belong to the category of regulated promoters. The inducible promoter can be induced by one or more conditions, such as a physical condition, microenvironment of the host cells, or the physiological state of the host cells, an inducer (i.e., an inducing agent), or a combination thereof. In some embodiments, the inducing condition does not induce the expression of endogenous genes in the host cell. In some embodiments, the inducing condition is selected from the group consisting of: inducer, irradiation (such as ionizing radiation, light), temperature (such as heat), redox state, and the activation state of the host cell. In some embodiments, the inducible promoter can be an NFAT promoter, a TETON® promoter, or an NFκB promoter.

Prokaryotic host cells suitable for expressing the proteins of the present application include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In some embodiments, gram-negative cells are used. In some embodiments, E. coli cells are used as hosts for the invention. Examples of E. coli strains include strain W3110 (Bachmann, Cellular and Molecular Biology, vol. 2 (Washington, D.C.: American Society for Microbiology, 1987), pp. 1190-1219; ATCC Deposit No. 27,325) and derivatives thereof, including strain 33D3 having genotype W3110 AfhuA (AtonA) ptr3 lac Iq lacL8 AompT A(nmpc-fepE) degP41 kan^{R}(U.S. Pat. No. 5,639,635). Other strains and derivatives thereof, such as E. coli 294 (ATCC 31,446), E. coli B, E. coli 1776 (ATCC 31,537) and E. coli RV308 (ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well-known plasmids such as pBR322, pBR325, pACYC177, or pKN410 are used to supply the replicon.

Suitable eukaryotic host cells for cloning or expressing the DNA in the vectors herein include vertebrate host cells. Propagation of vertebrate cells in culture (tissue culture) has become a routine procedure. Examples of useful mammalian host cell lines are monkey kidney CV1 line transformed by SV40 (COS-7, ATCC CRL 1651); COS fibroblast-like cell lines derived from monkey kidney tissue; human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for protein construct production and cultured in conventional nutrient media modified as appropriate for inducing promoters, selecting transformants, or amplifying the genes encoding the desired sequences.

Also provided are methods of preparing any of the FX activators (e.g., RVV-X) described herein. Thus in some embodiments, there is provided a method of producing an FX activator (e.g., RVV-X), comprising: (a) culturing a host cell (e.g., CHO cell) comprising any of the nucleic acids or vectors encoding the FX activators described herein under a condition effective to express the encoded FX activators; and (b) obtaining the expressed FX activator from said host cell. In some embodiments, the method of step (a) further comprises producing a host cell comprising the nucleic acid or vector encoding the FX activator (e.g., RVV-X) described herein. The FX activators (e.g., RVV-X) described herein may be prepared using any recombinant production methods known in the art, or isolated and/or purified (such as from venom).

In some embodiments, the FX activator (e.g., RVV-X) is isolated and/or purified from venom of snakes, such as snake species that belong to the genus Viperidae and Crotalidae as well as a few Elapid species. In some embodiments, the FX activator RVV-X is isolated and/or purified from Daboia nissellii siamensis venom. In some embodiments, the FX activator (e.g., RVV-X) is isolated and/or purified as described in CN109943554B. Any suitable protein purification methods can be used for FX activator purification, such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the protein construct to be recovered. In some embodiments, the FX activator is sterilized and/or inactivated for virus. In some embodiments, purification of the FX activator comprises one or more of viral inactivation, anion exchange chromatography, ultrafiltration, hydroxyapatite (HA) chromatography (HAC), and SEC.

### IV. Methods of Treating Bleeding Disorders

The FX activators (e.g., RVV-X) or pharmaceutical compositions thereof (e.g., RVV-X lead formulation) described herein are useful for a variety of applications, such as in diagnosis, molecular assays, or therapy (e.g., on-demand therapy, or preventive therapy). In some embodiments, the individual to be treated or diagnosed is a livestock (e.g., pig, sheep, goat, cow, ox, horse, donkey, mule, chicken, duck, goose). In some embodiments, the individual to be treated or diagnosed is a companion animal (e.g., pet) or assistive animal, such as dog, cat, rabbit, hamster, guinea pig, chinchilla, ferret, bird, etc. In some embodiments, the individual to be treated or diagnosed is a human. In some embodiments, the individual to be treated or diagnosed is a primate (e.g., monkey).

In some embodiments, there is provided a method of treating a bleeding disorder (e.g., hemophilia, such as hemophilia A or B, with or without inhibitor) in an individual (e.g., human), comprising administering to the individual an effective amount of any of the FX activators (e.g., RVV-X) or pharmaceutical compositions thereof (e.g., RVV-X lead formulation) described herein. In some embodiments, there is provided a method of treating a bleeding disorder (e.g., hemophilia, such as hemophilia A or B, with or without inhibitor) in an individual (e.g., human), comprising administering to the individual an effective amount of any of the FX activators (e.g., RVV-X) or pharmaceutical compositions thereof (e.g., RVV-X lead formulation) described herein, wherein the FX activator or pharmaceutical composition thereof is administered in a dose of from about 0.01 U/kg to about 0.48 U/kg (e.g., any of from about 0.08 U/kg to about 0.48 U/kg, from about 0.1 U/kg to about 0.48 U/kg, from about 0.01 U/kg to about 0.16 U/kg, from about 0.08 U/kg to about 0.16 U/kg, from about 0.1 U/kg to about 0.16 U/kg, or about 0.1 U/kg, 0.12 U/kg, 0.14 U/kg or 0.16 U/kg). In some embodiments, the FX activator or pharmaceutical composition thereof is administered intravenously, such as by intravenous injection. When referring to “FX activator pharmaceutical composition administered in a dose of X”, it means that the pharmaceutical composition is administered in an effective amount so that the FX activator contained therein is in a dose of X. In some embodiments, the FX activator or pharmaceutical composition thereof is administered once. In some embodiments, the FX activator or pharmaceutical composition thereof is administered for a maximum of 6 doses, such as any of 6, 5, 4, 3, 2, or 1 dose. In some embodiments, the FX activator or pharmaceutical composition thereof is administered for 4 doses. In some embodiments, the FX activator or pharmaceutical composition thereof is administered every 4 hours (q4h) to every 8 hours (q8h), such as any of every 4, 4.5, 5, 5.5, 6, 6.5, 7, 7.5, or 8 hours. In some embodiments, the FX activator or pharmaceutical composition thereof is administered q4h. In some embodiments, the FX activator or pharmaceutical composition thereof is administered once in a dose of from about 0.01 U/kg to about 0.48 U/kg (e.g., about any of 0.01 U/kg, 0.04 U/kg, 0.08 U/kg, 0.1 U/kg, 0.12 U/kg, 0.14 U/kg, 0.16 U/kg, 0.32 U/kg, or 0.48 U/kg). In some embodiments, the FX activator or pharmaceutical composition thereof is administered in a dose of about 0.16 U/kg q8 h for a maximum of 6 doses (e.g., any of 6, 5, 4, 3, 2, or 1 dose). In some embodiments, the FX activator or pharmaceutical composition thereof is administered in a dose of about 0.16 U/kg q8 h for 4 doses. In some embodiments, the FX activator or pharmaceutical composition thereof is administered in a dose of about 0.1 U/kg q8 h for a maximum of 6 doses (e.g., any of 6, 5, 4, 3, 2, or 1 dose). In some embodiments, the FX activator or pharmaceutical composition thereof is administered in a dose of about 0.1 U/kg q8 h for 4 doses. In some embodiments, the FX activator or pharmaceutical composition thereof is administered in a dose of about 0.16 U/kg q4 h for a maximum of 6 doses (e.g., any of 6, 5, 4, 3, 2, or 1 dose). In some embodiments, the FX activator or pharmaceutical composition thereof is administered in a dose of about 0.16 U/kg q4 h for 4 doses. In some embodiments, the FX activator or pharmaceutical composition thereof is administered in a dose of about 0.1 U/kg q4 h for a maximum of 6 doses (e.g., any of 6, 5, 4, 3, 2, or 1 dose). In some embodiments, the FX activator or pharmaceutical composition thereof is administered in a dose of about 0.1 U/kg q4 h for 4 doses. In some embodiments, the FX activator is RVV-X. In some embodiments, the RVV-X is isolated from Daboia russellii siamensis venom. In some embodiments, the purity of the RVV-X is at least about 95%. In some embodiments, the RVV-X comprises a) i) a heavy chain comprising the sequence of SEQ ID NO: 1; ii) a light chain 1 comprising the sequence of SEQ ID NO: 2; and iii) a light chain 2 comprising the sequence of SEQ ID NO: 3; b) i) a heavy chain comprising the sequence of SEQ ID NO: 1; ii) a light chain 1 comprising the sequence of SEQ ID NO: 5; and iii) a light chain 2 comprising the sequence of SEQ ID NO: 3; or c) a mixture of a) and b). In some embodiments, the bleeding disorder is hemophilia A, such as hemophilia A with or without FVIII inhibitor. In some embodiments, the bleeding disorder is hemophilia B, such as hemophilia B with or without IX inhibitor. In some embodiments, the bleeding disorder is surgical wound bleeding. In some embodiments, a consolidation therapy is further administered to the individual, such as one extra administration of the same dose of the FX activator or pharmaceutical composition thereof, e.g., within about 24 hours after the last administration in the treatment method. In some embodiments, the FX activator (e.g., RVV-X) or pharmaceutical compositions thereof (e.g., RVV-X lead formulation) is administered to the individual within about 24 hours (such as within about any of 20 hrs, 18 hrs, 16 hrs, 12 hrs, 10 hrs, 8 hrs, 6 hrs, 4 hrs, 2 hrs, 1 hr, 30 min, 20 min, 10 min, or shorter) since bleeding begins. In some embodiments, the FX activator (e.g., RVV-X) or pharmaceutical compositions thereof (e.g., RVV-X lead formulation) is administered to the individual at least about 5 minutes (such as at least about any of 10 min, 15 min, 20 min, 30 min, 40 min, 50 min, 1 hr, 2 hrs, or longer) before bleeding begins (e.g., before surgery operation). In some embodiments, the FX activator or pharmaceutical compositions thereof is administered to the individual from about 10 minutes to about 15 minutes before bleeding begins. In some embodiments, the individual is a human. In some embodiments, the individual is an animal, such as mouse, rat, rabbit, cat, dog, guinea pig, hamster, horse, cow, sheep, bird (e.g., chicken, duck), etc.

Dosages and desired drug concentration of pharmaceutical compositions of the present invention may vary depending on the particular use envisioned. The determination of the appropriate dosage or route of administration is well within the skill of an ordinary artisan. Interspecies scaling of effective doses can be performed following the principles laid down by Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

It is within the scope of the present application that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs (e.g., stop bleeding). However, other dosage regimens may be useful. The progress of this therapy is easily monitored by conventional techniques and assays.

In some embodiments, the FX activator (e.g., RVV-X) or pharmaceutical composition thereof (e.g., RVV-X lead formulation) described herein is administered for a single time (e.g. bolus injection). In some embodiments, the FX activator or pharmaceutical composition thereof is administered for multiple times (such as any of 2, 3, 4, 5, 6, or more times). If multiple administrations, they may be performed by the same or different routes and may take place at the same site or at alternative sites. The FX activator or pharmaceutical composition thereof may be administered hourly, daily, monthly, or on-demand. The interval between administrations can be about any one of 1 hour to 1 day. Intervals can also be irregular (e.g. following bleeding episode). In some embodiments, there is no break in the dosing schedule. The optimal dosage and treatment regime for a particular patient can readily be determined by one skilled in the art of medicine by monitoring the patient for signs of disease (e.g., bleeding signs) and adjusting the treatment accordingly. In some embodiments, the FX activator or pharmaceutical composition thereof is administered qlh, q2h, q4h, q6h, q8h, g12h, g16h, q18h, once per day (daily), once per 2 days, once per 3 days, once a week, once every 2 weeks, once every 3 weeks, once per month. In some embodiments, the FX activator or pharmaceutical composition thereof is administered q4 h to q8h.

In some embodiments, the FX activator (e.g., RVV-X) or pharmaceutical composition thereof (e.g., RVV-X lead formulation) described herein is administered in split doses, such as about any one of 2, 3, 4, 5, or 6 doses. In some embodiments, the split doses are administered over about 1 day, about 2 days, about 3 days, about a week, about a month, or longer. In some embodiments, the dose is equally split. In some embodiments, the split doses are about 17%, about 20%, about 25%, about 30%, or about 50% of the total dose. In some embodiments, the interval between consecutive split doses is about 2 hrs, 4 hrs, 6 hrs, 8 hrs, 10 hrs, 12 hrs, 18 hrs, 1 day, 2 days, 3 days, 1 week, I month, or longer. For repeated administrations over several hours, days, or longer, depending on the condition, the treatment is sustained until a desired suppression of disease symptoms occurs. However, other dosage regimens may be useful. The progress of this therapy is easily monitored by conventional techniques and assays.

In some embodiments, the FX activator (e.g., RVV-X) or pharmaceutical composition thereof (e.g., RVV-X lead formulation) is administered in a dose of from about 0.01 U/kg to about 0.48 U/kg, such as any of from about 0.01 U/kg to about 0.1 U/kg, from about 0.02 U/kg to about 0.08 U/kg, from about 0.01 U/kg to about 0.05 U/kg, from about 0.01 U/kg to about 0.08 U/kg, from about 0.05 U/kg to about 0.1 U/kg, from about 0.1 U/kg to about 0.48 U/kg, from about 0.1 U/kg to about 0.4 U/kg, from about 0.1 U/kg to about 0.3 U/kg, from about 0.1 U/kg to about 0.2 U/kg, from about 0.2 U/kg to about 0.48 U/kg, from about 0.2 U/kg to about 0.4 U/kg, from about 0.2 U/kg to about 0.3 U/kg, from about 0.3 U/kg to about 0.48 U/kg, from about 0.08 U/kg to about 0.48 U/kg, from about 0.16 U/kg to about 0.48 U/kg, from about 0.16 U/kg to about 0.2 U/kg, from about 0.01 U/kg to about 0.16 U/kg, from about 0.02 U/kg to about 0.14 U/kg, from about 0.04 U/kg to about 0.12 U/kg, from about 0.06 U/kg to about 0.12 U/kg, from about 0.08 U/kg to about 0.1 U/kg, from about 0.02 U/kg to about 0.16 U/kg, from about 0.04 U/kg to about 0.16 U/kg, from about 0.06 U/kg to about 0.16 U/kg, from about 0.1 U/kg to about 0.16 U/kg, from about 0.12 U/kg to about 0.16 U/kg, from about 0.14 U/kg to about 0.16 U/kg, from about 0.08 U/kg to about 0.16 U/kg, or from about 0.1 U/kg to about 0.16 U/kg. In some embodiments, the individual to be treated is a human. In some embodiments, the FX activator (e.g., RVV-X) or pharmaceutical composition thereof is administered in a dose of about any of 0.01 U/kg, 0.02 U/kg, 0.03 U/kg, 0.04 U/kg, 0.05 U/kg, 0.06 U/kg, 0.07 U/kg, 0.08 U/kg, 0.09 U/kg, 0.1 U/kg, 0.11 U/kg, 0.12 U/kg, 0.13 U/kg, 0.14 U/kg, 0.15 U/kg, 0.16 U/kg, 0.18 U/kg, 0.2 U/kg, 0.22 U/kg, 0.24 U/kg, 0.26 U/kg, 0.28 U/kg, 0.3 U/kg, 0.32 U/kg, 0.36 U/kg, 0.4 U/kg, 0.44 U/kg, and 0.48 U/kg, such as about 0.16 U/kg.

In some embodiments, there is provided a method of treating hemophilia (e.g., hemophilia A or B, with or without inhibitor) in an individual (e.g., human), comprising administering to the individual an effective amount of RVV-X or pharmaceutical composition thereof (e.g., RVV-X lead formulation), wherein the RVV-X or pharmaceutical composition thereof is administered once in a dose of any of from about 0.01 U/kg to about 0.48 U/kg, from about 0.16 U/kg to about 0.48 U/kg, from about 0.08 U/kg to about 0.48 U/kg, from about 0.1 U/kg to about 0.48 U/kg, from about 0.01 U/kg to about 0.16 U/kg once, from about 0.08 U/kg to about 0.16 U/kg, from about 0.1 U/kg to about 0.16 U/kg, or about 0.1 U/kg, 0.12 U/kg, 0.14 U/kg or 0.16 U/kg. In some embodiments, there is provided a method of treating hemophilia (e.g., hemophilia A or B, with or without inhibitor) in an individual (e.g., human), comprising administering to the individual an effective amount of RVV-X or pharmaceutical composition thereof (e.g., RVV-X lead formulation), wherein the RVV-X or pharmaceutical composition thereof is administered once in a dose of from about 0.01 U/kg to about 0.48 U/kg, from about 0.1 U/kg to about 0.48 U/kg, or from about 0.16 U/kg to about 0.48 U/kg. In some embodiments, there is provided a method of treating hemophilia (e.g., hemophilia A or B, with or without inhibitor) in an individual (e.g., human), comprising administering to the individual an effective amount of RVV-X or pharmaceutical composition thereof (e.g., RVV-X lead formulation), wherein the RVV-X or pharmaceutical composition thereof is administered once in a dose of about any of 0.01 U/kg, 0.04 U/kg, 0.08 U/kg, 0.1 U/kg, 0.12 U/kg, 0.14 U/kg, 0.16 U/kg, 0.32 U/kg, and 0.48 U/kg. In some embodiments, there is provided a method of treating hemophilia (e.g., hemophilia A or B, with or without inhibitor) in an individual (e.g., human), comprising administering to the individual an effective amount of RVV-X or pharmaceutical composition thereof (e.g., RVV-X lead formulation), wherein the RVV-X or pharmaceutical composition thereof is administered once in a dose of about any of 0.1 U/kg, 0.12 U/kg, 0.14 U/kg, 0.16 U/kg, 0.32 U/kg, or 0.48 U/kg. In some embodiments, the RVV-X or pharmaceutical composition thereof (e.g., RVV-X lead formulation) is administered intravenously, such as by intravenous injection. In some embodiments, the RVV-X is isolated from Daboia russellii siamensis venom. In some embodiments, the purity of the RVV-X is at least about 95%. In some embodiments, the RVV-X comprises a) i) a heavy chain comprising the sequence of SEQ ID NO: 1; ii) a light chain 1 comprising the sequence of SEQ ID NO: 2; and iii) a light chain 2 comprising the sequence of SEQ ID NO: 3; b) i) a heavy chain comprising the sequence of SEQ ID NO: 1; ii) a light chain 1 comprising the sequence of SEQ ID NO: 5; and iii) a light chain 2 comprising the sequence of SEQ ID NO: 3; or c) a mixture of a) and b).

In some embodiments, there is provided a method of treating hemophilia (e.g., hemophilia A or B, with or without inhibitor) in an individual (e.g., human), comprising administering to the individual an effective amount of a pharmaceutical composition; wherein the pharmaceutical composition comprises i) an FX activator (e.g., RVV-X) in an amount of from about 0.1 U/mL to about 200 U/mL (e.g., from about 1 U/mL to about 100 U/mL, from about 5 U/mL to about 100 U/mL, from about 5 U/mL to about 50 U/mL, or about 10 U/mL); ii) a stabilizer (e.g., sucrose) in an amount of from about 2 mg/ml to about 100 mg/ml (e.g., from about 2 mg/ml to about 60 mg/ml, from about 15 mg/ml to about 60 mg/ml, from about 30 mg/ml to about 50 mg/ml, or about 30 mg/ml); iii) a buffering agent (e.g., histidine) in an amount of from about 0.1 mg/ml to about 50 mg/ml (e.g., from about 2 mg/ml to about 20 mg/ml, from about 2 mg/ml to about 15 mg/ml, from about 3 mg/ml to about 5 mg/ml, or about 3 mg/ml); iv) a surfactant (e.g., polysorbate 20) in an amount of from about 0.001% (w/v) to about 0.1% (w/v) (e.g., from about 0.005% (w/v) to about 0.05% (w/v), from about 0.01% (w/v) to about 0.05% (w/v), from about 0.01% (w/v) to about 0.03% (w/v), or about 0.02% (w/v)); and v) a tonicity agent (e.g., mannitol) in an amount of from about 1 mg/ml to about 100 mg/ml (e.g., from about 10 mg/ml to about 60 mg/ml, from about 30 mg/ml to about 60 mg/ml, or about 40 mg/ml); wherein the pharmaceutical composition has a pH of from about 6.0 to about 8.0 (e.g., from about 6.3 to about 7.3, from about 6.8 to about 7.0, or about 6.85); and wherein the pharmaceutical composition is administered once in a dose of any of from about 0.01 U/kg to about 0.48 U/kg, from about 0.16 U/kg to about 0.48 U/kg, from about 0.08 U/kg to about 0.48 U/kg, from about 0.1 U/kg to about 0.48 U/kg, from about 0.01 U/kg to about 0.16 U/kg once, from about 0.08 U/kg to about 0.16 U/kg, from about 0.1 U/kg to about 0.16 U/kg, or about 0.1 U/kg, 0.12 U/kg, 0.14 U/kg or about 0.16 U/kg. In some embodiments, there is provided a method of treating hemophilia (e.g., hemophilia A or B, with or without inhibitor) in an individual (e.g., human), comprising administering to the individual an effective amount of a pharmaceutical composition; wherein the pharmaceutical composition comprises i) an FX activator (e.g., RVV-X) in an amount of from about 0.1 U/mL to about 200 U/mL (e.g., from about 1 U/mL to about 100 U/mL, from about 5 U/mL to about 100 U/mL, from about 5 U/mL to about 50 U/mL, or about 10 U/mL); ii) a stabilizer (e.g., sucrose) in an amount of from about 2 mg/ml to about 100 mg/ml (e.g., from about 2 mg/ml to about 60 mg/ml, from about 15 mg/ml to about 60 mg/ml, from about 30 mg/ml to about 50 mg/ml, or about 30 mg/ml); iii) a buffering agent (e.g., histidine) in an amount of from about 0.1 mg/ml to about 50 mg/ml (e.g., from about 2 mg/ml to about 20 mg/ml, from about 2 mg/ml to about 15 mg/ml, from about 3 mg/ml to about 5 mg/ml, or about 3 mg/ml); iv) a surfactant (e.g., polysorbate 20) in an amount of from about 0.001% (w/v) to about 0.1% (w/v) (e.g., from about 0.005% (w/v) to about 0.05% (w/v), from about 0.01% (w/v) to about 0.05% (w/v), from about 0.01% (w/v) to about 0.03% (w/v), or about 0.02% (w/v)); and v) a tonicity agent (e.g., mannitol) in an amount of from about 1 mg/ml to about 100 mg/ml (e.g., from about 10 mg/ml to about 60 mg/ml, from about 30 mg/ml to about 60 mg/ml, or about 40 mg/ml); wherein the pharmaceutical composition has a pH of from about 6.0 to about 8.0 (e.g., from about 6.3 to about 7.3, from about 6.8 to about 7.0, or about 6.85); and wherein the pharmaceutical composition is administered once in a dose of from about 0.01 U/kg to about 0.48 U/kg, from about 0. U/kg to about 0.48 U/kg or from about 0.16 U/kg to about 0.48 U/kg. In some embodiments, there is provided a method of treating hemophilia (e.g., hemophilia A or B, with or without inhibitor) in an individual (e.g., human), comprising administering to the individual an effective amount of a pharmaceutical composition; wherein the pharmaceutical composition comprises i) an FX activator (e.g., RVV-X) in an amount of from about 0.1 U/mL to about 200 U/mL (e.g., from about 1 U/mL to about 100 U/mL, from about 5 U/mL to about 100 U/mL, from about 5 U/mL to about 50 U/mL, or about 10 U/mL); ii) a stabilizer (e.g., sucrose) in an amount of from about 2 mg/ml to about 100 mg/ml (e.g., from about 2 mg/ml to about 60 mg/ml, from about 15 mg/ml to about 60 mg/ml, from about 30 mg/ml to about 50 mg/ml, or about 30 mg/ml); iii) a buffering agent (e.g., histidine) in an amount of from about 0.1 mg/ml to about 50 mg/ml (e.g., from about 2 mg/ml to about 20 mg/ml, from about 2 mg/ml to about 15 mg/ml, from about 3 mg/ml to about 5 mg/ml, or about 3 mg/ml); iv) a surfactant (e.g., polysorbate 20) in an amount of from about 0.001% (w/v) to about 0.1% (w/v) (e.g., from about 0.005% (w/v) to about 0.05% (w/v), from about 0.01% (w/v) to about 0.05% (w/v), from about 0.01% (w/v) to about 0.03% (w/v), or about 0.02% (w/v)); and v) a tonicity agent (e.g., mannitol) in an amount of from about 1 mg/ml to about 100 mg/ml (e.g., from about 10 mg/ml to about 60 mg/ml, from about 30 mg/ml to about 60 mg/ml, or about 40 mg/ml); wherein the pharmaceutical composition has a pH of from about 6.0 to about 8.0 (e.g., from about 6.3 to about 7.3, from about 6.8 to about 7.0, or about 6.85); and wherein the pharmaceutical composition is administered once in a dose of about any of 0.01 U/kg, 0.04 U/kg, 0.08 U/kg, 0.1 U/kg, 0.12 U/kg, 0.14 U/kg, 0.16 U/kg, 0.32 U/kg, and 0.48 U/kg. In some embodiments, there is provided a method of treating hemophilia (e.g., hemophilia A or B, with or without inhibitor) in an individual (e.g., human), comprising administering to the individual an effective amount of a pharmaceutical composition; wherein the pharmaceutical composition comprises i) an FX activator (e.g., RVV-X) in an amount of from about 0.1 U/mL to about 200 U/mL (e.g., from about 1 U/mL to about 100 U/mL, from about 5 U/mL to about 100 U/mL, from about 5 U/mL to about 50 U/mL, or about 10 U/mL); ii) a stabilizer (e.g., sucrose) in an amount of from about 2 mg/ml to about 100 mg/ml (e.g., from about 2 mg/ml to about 60 mg/ml, from about 15 mg/ml to about 60 mg/ml, from about 30 mg/ml to about 50 mg/ml, or about 30 mg/ml); iii) a buffering agent (e.g., histidine) in an amount of from about 0.1 mg/ml to about 50 mg/ml (e.g., from about 2 mg/ml to about 20 mg/ml, from about 2 mg/ml to about 15 mg/ml, from about 3 mg/ml to about 5 mg/ml, or about 3 mg/ml); iv) a surfactant (e.g., polysorbate 20) in an amount of from about 0.001% (w/v) to about 0.1% (w/v) (e.g., from about 0.005% (w/v) to about 0.05% (w/v), from about 0.01% (w/v) to about 0.05% (w/v), from about 0.01% (w/v) to about 0.03% (w/v), or about 0.02% (w/v)); and v) a tonicity agent (e.g., mannitol) in an amount of from about 1 mg/ml to about 100 mg/ml (e.g., from about 10 mg/ml to about 60 mg/ml, from about 30 mg/ml to about 60 mg/ml, or about 40 mg/ml); wherein the pharmaceutical composition has a pH of from about 6.0 to about 8.0 (e.g., from about 6.3 to about 7.3, from about 6.8 to about 7.0, or about 6.85); and wherein the pharmaceutical composition is administered once in a dose of about any of 0.1 U/kg, 0.12 U/kg, 0.14 U/kg, 0.16 U/kg, 0.32 U/kg, or 0.48 U/kg. In some embodiments, the pharmaceutical composition is lyophilized. In some embodiments, the pharmaceutical composition is sterile. In some embodiments, the method further comprises reconstituting the pharmaceutical composition (e.g., with 0.9% sodium chloride injection) before administration. In some embodiments, the pharmaceutical composition is administered intravenously, such as by intravenous injection. In some embodiments, the FX activator is RVV-X. In some embodiments, the RVV-X is isolated from Daboia russellii siamensis venom. In some embodiments, the purity of the RVV-X is at least about 95%. In some embodiments, the RVV-X comprises a) i) a heavy chain comprising the sequence of SEQ ID NO: 1, or a sequence with at least about 80% identity to SEQ ID NO: 1; ii) a light chain 1 comprising the sequence of SEQ ID NO: 2, or a sequence with at least about 80% identity to SEQ ID NO: 2; and iii) a light chain 2 comprising the sequence of SEQ ID NO: 3, or a sequence with at least about 80% identity to SEQ ID NO: 3; b) i) a heavy chain comprising the sequence of SEQ ID NO: 1, or a sequence with at least about 8004 identity to SEQ ID NO: 1; ii) a light chain 1 comprising the sequence of SEQ ID NO: 5, or a sequence with at least about 80% identity to SEQ ID NO: 5; and iii) a light chain 2 comprising the sequence of SEQ ID NO: 3, or a sequence with at least about 80% identity to SEQ ID NO: 3; or c) a mixture of a) and b).

In some embodiments, there is provided a method of treating hemophilia (e.g., hemophilia A or B, with or without inhibitor) in an individual (e.g., human), comprising administering to the individual an effective amount of a pharmaceutical composition; wherein the pharmaceutical composition comprises i) RVV-X in an amount of about 10 U/mL, ii) sucrose in an amount of about 30 mg/ml, iii) histidine in an amount of about 3 mg/ml, iv) polysorbate 20 in an amount of about 0.02% (w/v), and v) mannitol in an amount of about 40 mg/ml, wherein the pharmaceutical composition has a pH of about 6.85; and wherein the pharmaceutical composition is administered once in a dose of any of from about 0.01 U/kg to about 0.48 U/kg, from about 0.16 U/kg to about 0.48 U/kg, from about 0.08 U/kg to about 0.48 U/kg, from about 0.1 U/kg to about 0.48 U/kg, from about 0.01 U/kg to about 0.16 U/kg once, from about 0.08 U/kg to about 0.16 U/kg, from about 0.1 U/kg to about 0.16 U/kg, or about 0.1 U/kg, 0.12 U/kg, 0.14 U/kg or 0.16 U/kg. In some embodiments, there is provided a method of treating hemophilia (e.g., hemophilia A or B, with or without inhibitor) in an individual (e.g., human), comprising administering to the individual an effective amount of a pharmaceutical composition; wherein the pharmaceutical composition comprises i) RVV-X in an amount of about 10 U/mL, ii) sucrose in an amount of about 30 mg/ml, iii) histidine in an amount of about 3 mg/ml, iv) polysorbate in an amount of about 0.02% (w/v), and v) mannitol in an amount of about 40 mg/ml, wherein the pharmaceutical composition has a pH of about 6.85; and wherein the pharmaceutical composition is administered once in a dose of from about 0.01 U/kg to about 0.48 U/kg, from about 0.1 U/kg to about 0.48 U/kg, or from about 0.16 U/kg to about 0.48 U/kg. In some embodiments, there is provided a method of treating hemophilia (e.g., hemophilia A or B, with or without inhibitor) in an individual (e.g., human), comprising administering to the individual an effective amount of a pharmaceutical composition; wherein the pharmaceutical composition comprises i) RVV-X in an amount of about 10 U/mL, ii) sucrose in an amount of about 30 mg/ml, iii) histidine in an amount of about 3 mg/ml, iv) polysorbate 20 in an amount of about 0.02% (w/v), and v) mannitol in an amount of about 40 mg/ml, wherein the pharmaceutical composition has a pH of about 6.85; and wherein the pharmaceutical composition is administered once in a dose of about any of 0.01 U/kg, 0.04 U/kg, 0.08 U/kg, 0.1 U/kg, 0.12 U/kg, 0.14 U/kg, 0.16 U/kg, 0.32 U/kg, and 0.48 U/kg. In some embodiments, there is provided a method of treating hemophilia (e.g., hemophilia A or B, with or without inhibitor) in an individual (e.g., human), comprising administering to the individual an effective amount of a pharmaceutical composition; wherein the pharmaceutical composition comprises i) RVV-X in an amount of about 10 U/mL, ii) sucrose in an amount of about 30 mg/ml, iii) histidine in an amount of about 3 mg/ml, iv) polysorbate 20 in an amount of about 0.02% (w/v), and v) mannitol in an amount of about 40 mg/ml, wherein the pharmaceutical composition has a pH of about 6.85; and wherein the pharmaceutical composition is administered once in a dose of about any of 0.1 U/kg, 0.12 U/kg, 0.14 U/kg, 0.16 U/kg, 0.32 U/kg, or 0.48 U/kg. In some embodiments, the pharmaceutical composition is lyophilized. In some embodiments, the pharmaceutical composition is sterile. In some embodiments, the method further comprises reconstituting the pharmaceutical composition (e.g., with 0.9% sodium chloride injection) before administration. In some embodiments, the pharmaceutical composition is administered intravenously, such as by intravenous injection. In some embodiments, the RVV-X is isolated from Daboia russellii siamensis venom. In some embodiments, the purity of the RVV-X is at least about 95%. In some embodiments, the RVV-X comprises a) i) a heavy chain comprising the sequence of SEQ ID NO: 1; ii) a light chain 1 comprising the sequence of SEQ ID NO: 2; and iii) a light chain 2 comprising the sequence of SEQ ID NO: 3; b) i) a heavy chain comprising the sequence of SEQ ID NO: 1; ii) a light chain 1 comprising the sequence of SEQ ID NO: 5; and iii) a light chain 2 comprising the sequence of SEQ ID NO: 3; or c) a mixture of a) and b).

In some embodiments, there is provided a method of treating hemophilia (e.g., hemophilia A or B, with or without inhibitor) in an individual (e.g., human), comprising administering to the individual an effective amount of RVV-X or pharmaceutical composition thereof (e.g., RVV-X lead formulation), wherein the RVV-X or pharmaceutical composition thereof is administered in a dose of from about 0.01 U/kg to about 0.48 U/kg (such as any of from about 0.16 U/kg to about 0.48 U/kg, from about 0.08 U/kg to about 0.48 U/kg, from about 0.1 U/kg to about 0.48 U/kg, from about 0.01 U/kg to about 0.16 U/kg once, from about 0.08 U/kg to about 0.16 U/kg, from about 0.1 U/kg to about 0.16 U/kg or about 0.1 U/kg or 0.16 U/kg), q4 h to q8h (such as any of q4h, q5h, q6h, q7h, or q8h), for a maximum of 6 doses (such as 1, 2, 3, 4, 5, or 6 doses). In some embodiments, there is provided a method of treating hemophilia (e.g., hemophilia A or B, with or without inhibitor) in an individual (e.g., human), comprising administering to the individual an effective amount of RVV-X or pharmaceutical composition thereof (e.g., RVV-X lead formulation), wherein the RVV-X or pharmaceutical composition thereof is administered in a dose of about 0.1 U/kg or 0.16 U/kg, q4 h to q8h (such as any of q4h, q5h, q6h, q7h, or q8h), for a maximum of 6 doses (such as 1, 2, 3, 4, 5, or 6 doses). In some embodiments, there is provided a method of treating hemophilia (e.g., hemophilia A or B, with or without inhibitor) in an individual (e.g., human), comprising administering to the individual an effective amount of RVV-X or pharmaceutical composition thereof (e.g., RVV-X lead formulation), wherein the RVV-X or pharmaceutical composition thereof is administered in a dose of about 0.1 U/kg or 0.16 U/kg q4 h for a maximum of 6 doses (such as 1, 2, 3, 4, 5, or 6 doses). In some embodiments, there is provided a method of treating hemophilia (e.g., hemophilia A or B, with or without inhibitor) in an individual (e.g., human), comprising administering to the individual an effective amount of RVV-X or pharmaceutical composition thereof (e.g., RVV-X lead formulation), wherein the RNV-X or pharmaceutical composition thereof is administered in a dose of about 0.1 U/kg or 0.16 U/kg q4 h for 4 doses. In some embodiments, the RVV-X or pharmaceutical composition thereof (e.g., RVV-X lead formulation) is administered intravenously, such as by intravenous injection. In some embodiments, the RVV-X is isolated from Daboia russellii siamensis venom. In some embodiments, the purity of the RVV-X is at least about 95%. In some embodiments, the RVV-X comprises a) i) a heavy chain comprising the sequence of SEQ ID NO: 1; ii) a light chain 1 comprising the sequence of SEQ ID NO: 2; and iii) a light chain 2 comprising the sequence of SEQ ID NO: 3; b) i) a heavy chain comprising the sequence of SEQ ID NO: 1; ii) a light chain 1 comprising the sequence of SEQ ID NO: 5; and iii) a light chain 2 comprising the sequence of SEQ ID NO: 3; or c) a mixture of a) and b).

In some embodiments, there is provided a method of treating hemophilia (e.g., hemophilia A or B, with or without inhibitor) in an individual (e.g., human), comprising administering to the individual an effective amount of a pharmaceutical composition; wherein the pharmaceutical composition comprises i) an FX activator (e.g., RVV-X) in an amount of from about 0.1 U/mL to about 200 U/mL (e.g., from about 1 U/mL to about 100 U/mL, from about 5 U/mL to about 100 U/mL, from about 5 U/mL to about 50 U/mL, or about 10 U/mL); ii) a stabilizer (e.g., sucrose) in an amount of from about 2 mg/ml to about 100 mg/ml (e.g., from about 2 mg/ml to about 60 mg/ml, from about 15 mg/ml to about 60 mg/ml, from about 30 mg/ml to about 50 mg/ml, or about 30 mg/ml); iii) a buffering agent (e.g., histidine) in an amount of from about 0.1 mg/ml to about 50 mg/ml (e.g., from about 2 mg/ml to about 20 mg/ml, from about 2 mg/ml to about 15 mg/ml, from about 3 mg/ml to about 5 mg/ml, or about 3 mg/ml); iv) a surfactant (e.g., polysorbate 20) in an amount of from about 0.001% (w/v) to about 0.1% (w/v) (e.g., from about 0.005% (w/v) to about 0.05% (w/v), from about 0.01% (w/v) to about 0.05% (w/v), from about 0.01% (w/v) to about 0.03% (w/v), or about 0.02% (w/v)); and v) a tonicity agent (e.g., mannitol) in an amount of from about 1 mg/ml to about 100 mg/ml (e.g., from about 10 mg/ml to about 60 mg/ml, from about 30 mg/ml to about 60 mg/ml, or about 40 mg/ml); wherein the pharmaceutical composition has a pH of from about 6.0 to about 8.0 (e.g., from about 6.3 to about 7.3, from about 6.8 to about 7.0, or about 6.85); and wherein the pharmaceutical composition is administered in a dose of from about 0.01 U/kg to about 0.48 U/kg (such as any of from about 0.16 U/kg to about 0.48 U/kg, from about 0.08 U/kg to about 0.48 U/kg, from about 0.1 U/kg to about 0.48 U/kg, from about 0.01 U/kg to about 0.16 U/kg once, from about 0.08 U/kg to about 0.16 U/kg, from about 0.1 U/kg to about 0.16 U/kg, or about 0.1 U/kg or 0.16 U/kg), q4 h to q8h (such as any of q4h, q5h, q6h, q7h, or q8h), for a maximum of 6 doses (such as 1, 2, 3, 4, 5, or 6 doses). In some embodiments, there is provided a method of treating hemophilia (e.g., hemophilia A or B, with or without inhibitor) in an individual (e.g., human), comprising administering to the individual an effective amount of a pharmaceutical composition; wherein the pharmaceutical composition comprises i) an FX activator (e.g., RVV-X) in an amount of from about 0.1 U/mL to about 200 U/mL (e.g., from about 1 U/mL to about 100 U/mL, from about 5 U/mL to about 100 U/mL, from about 5 U/mL to about 50 U/mL, or about 10 U/mL); ii) a stabilizer (e.g., sucrose) in an amount of from about 2 mg/ml to about 100 mg/ml (e.g., from about 2 mg/ml to about 60 mg/ml, from about 15 mg/ml to about 60 mg/ml, from about 30 mg/ml to about 50 mg/ml, or about 30 mg/ml); iii) a buffering agent (e.g., histidine) in an amount of from about 0.1 mg/ml to about 50 mg/ml (e.g., from about 2 mg/ml to about 20 mg/ml, from about 2 mg/ml to about 15 mg/ml, from about 3 mg/ml to about 5 mg/ml, or about 3 mg/ml); iv) a surfactant (e.g., polysorbate 20) in an amount of from about 0.001% (w/v) to about 0.1% (w/v) (e.g., from about 0.005% (w/v) to about 0.05% (w/v), from about 0.01% (w/v) to about 0.05% (w/v), from about 0.01% (w/v) to about 0.03% (w/v), or about 0.02% (w/v)); and v) a tonicity agent (e.g., mannitol) in an amount of from about 1 mg/ml to about 100 mg/ml (e.g., from about 10 mg/ml to about 60 mg/ml, from about 30 mg/ml to about 60 mg/ml, or about 40 mg/ml); wherein the pharmaceutical composition has a pH of from about 6.0 to about 8.0 (e.g., from about 6.3 to about 7.3, from about 6.8 to about 7.0, or about 6.85); and wherein the pharmaceutical composition is administered in a dose of about 0.1 U/kg or 0.16 U/kg, q4 h to q8h (such as any of q4h, q5h, q6h, q7h, or q8h), for a maximum of 6 doses (such as 1, 2, 3, 4, 5, or 6 doses). In some embodiments, there is provided a method of treating hemophilia (e.g., hemophilia A or B, with or without inhibitor) in an individual (e.g., human), comprising administering to the individual an effective amount of a pharmaceutical composition; wherein the pharmaceutical composition comprises i) an FX activator (e.g., RVV-X) in an amount of from about 0.1 U/mL to about 200 U/mL (e.g., from about 1 U/mL to about 100 U/mL, from about 5 U/mL to about 100 U/mL, from about 5 U/mL to about 50 U/mL, or about 10 U/mL); ii) a stabilizer (e.g., sucrose) in an amount of from about 2 mg/ml to about 100 mg/ml (e.g., from about 2 mg/ml to about 60 mg/ml, from about 15 mg/ml to about 60 mg/ml, from about 30 mg/ml to about 50 mg/ml, or about 30 mg/ml); iii) a buffering agent (e.g., histidine) in an amount of from about 0.1 mg/ml to about 50 mg/ml (e.g., from about 2 mg/ml to about 20 mg/ml, from about 2 mg/ml to about 15 mg/ml, from about 3 mg/ml to about 5 mg/ml, or about 3 mg/ml); iv) a surfactant (e.g., polysorbate 20) in an amount of from about 0.001% (w/v) to about 0.1% (w/v) (e.g., from about 0.005% (w/v) to about 0.05% (w/v), from about 0.01% (w/v) to about 0.05% (w/v), from about 0.01% (w/v) to about 0.03% (w/v), or about 0.02% (w/v)); and v) a tonicity agent (e.g., mannitol) in an amount of from about 1 mg/ml to about 100 mg/ml (e.g., from about 10 mg/ml to about 60 mg/ml, from about 30 mg/ml to about 60 mg/ml, or about 40 mg/ml); wherein the pharmaceutical composition has a pH of from about 6.0 to about 8.0 (e.g., from about 6.3 to about 7.3, from about 6.8 to about 7.0, or about 6.85); and wherein the pharmaceutical composition is administered in a dose of about 0.1 U/kg or 0.16 U/kg q4 h for a maximum of 6 doses (such as 1, 2, 3, 4, 5, or 6 doses). In some embodiments, there is provided a method of treating hemophilia (e.g., hemophilia A or B, with or without inhibitor) in an individual (e.g., human), comprising administering to the individual an effective amount of a pharmaceutical composition; wherein the pharmaceutical composition comprises i) an FX activator (e.g., RVV-X) in an amount of from about 0.1 U/mL to about 200 U/mL (e.g., from about 1 U/mL to about 100 U/mL, from about 5 U/mL to about 100 U/mL, from about 5 U/mL to about 50 U/mL, or about 10 U/mL); ii) a stabilizer (e.g., sucrose) in an amount of from about 2 mg/ml to about 100 mg/ml (e.g., from about 2 mg/ml to about 60 mg/ml, from about 15 mg/ml to about 60 mg/ml, from about 30 mg/ml to about 50 mg/ml, or about 30 mg/ml); iii) a buffering agent (e.g., histidine) in an amount of from about 0.1 mg/ml to about 50 mg/ml (e.g., from about 2 mg/ml to about 20 mg/ml, from about 2 mg/ml to about 15 mg/ml, from about 3 mg/ml to about 5 mg/ml, or about 3 mg/ml); iv) a surfactant (e.g., polysorbate 20) in an amount of from about 0.001% (w/v) to about 0.1% (w/v) (e.g., from about 0.005% (w/v) to about 0.05% (w/v), from about 0.01% (w/v) to about 0.05% (w/v), from about 0.01% (w/v) to about 0.03% (w/v), or about 0.02% (w/v)); and v) a tonicity agent (e.g., mannitol) in an amount of from about 1 mg/ml to about 100 mg/ml (e.g., from about 10 mg/ml to about 60 mg/ml, from about 30 mg/ml to about 60 mg/ml, or about 40 mg/ml); wherein the pharmaceutical composition has a pH of from about 6.0 to about 8.0 (e.g., from about 6.3 to about 7.3, from about 6.8 to about 7.0, or about 6.85); and wherein the pharmaceutical composition is administered in a dose of about 0.1 U/kg or 0.16 U/kg q4 h for 4 doses. In some embodiments, the pharmaceutical composition is lyophilized. In some embodiments, the pharmaceutical composition is sterile. In some embodiments, the method further comprises reconstituting the pharmaceutical composition (e.g., with 0.9% sodium chloride injection) before administration. In some embodiments, the pharmaceutical composition is administered intravenously, such as by intravenous injection. In some embodiments, the FX activator is RVV-X. In some embodiments, the RVV-X is isolated from Daboia russellii siamensis venom. In some embodiments, the purity of the RVV-X is at least about 95%. In some embodiments, the RVV-X comprises a) i) a heavy chain comprising the sequence of SEQ ID NO: 1, or a sequence with at least about 80% identity to SEQ ID NO: 1; ii) a light chain 1 comprising the sequence of SEQ ID NO: 2, or a sequence with at least about 80% identity to SEQ ID NO: 2; and iii) a light chain 2 comprising the sequence of SEQ ID NO: 3, or a sequence with at least about 80% identity to SEQ ID NO: 3; b) i) a heavy chain comprising the sequence of SEQ ID NO: 1, or a sequence with at least about 80% identity to SEQ ID NO: 1; ii) a light chain 1 comprising the sequence of SEQ ID NO: 5, or a sequence with at least about 80% identity to SEQ ID NO: 5; and iii) a light chain 2 comprising the sequence of SEQ ID NO: 3, or a sequence with at least about 80% identity to SEQ ID NO: 3; or c) a mixture of a) and b).

In some embodiments, there is provided a method of treating hemophilia (e.g., hemophilia A or B, with or without inhibitor) in an individual (e.g., human), comprising administering to the individual an effective amount of a pharmaceutical composition; wherein the pharmaceutical composition comprises i) RVV-X in an amount of about 10 U/mL, ii) sucrose in an amount of about 30 mg/ml, iii) histidine in an amount of about 3 mg/ml, iv) polysorbate 20 in an amount of about 0.02% (w/v), and v) mannitol in an amount of about 40 mg/ml, wherein the pharmaceutical composition has a pH of about 6.85; and wherein the pharmaceutical composition is administered in a dose of from about 0.01 U/kg to about 0.48 U/kg (such as any of from about 0.16 U/kg to about 0.48 U/kg, from about 0.08 U/kg to about 0.48 U/kg, from about 0.1 U/kg to about 0.48 U/kg, from about 0.01 U/kg to about 0.16 U/kg once, from about 0.08 U/kg to about 0.16 U/kg, from about 0.1 U/kg to about 0.16 U/kg, about 0.1 U/kg or 0.16 U/kg), q4 h to q8h (such as any of q4h, q5h, q6h, q7h, or q8h), for a maximum of 6 doses (such as 1, 2, 3, 4, 5, or 6 doses). In some embodiments, there is provided a method of treating hemophilia (e.g., hemophilia A or B, with or without inhibitor) in an individual (e.g., human), comprising administering to the individual an effective amount of a pharmaceutical composition; wherein the pharmaceutical composition comprises i) RVV-X in an amount of about 10 U/mL, ii) sucrose in an amount of about 30 mg/ml, iii) histidine in an amount of about 3 mg/ml, iv) polysorbate 20 in an amount of about 0.02% (w/v), and v) mannitol in an amount of about 40 mg/ml, wherein the pharmaceutical composition has a pH of about 6.85; and wherein the pharmaceutical composition is administered in a dose of about 0.1 U/kg or 0.16 U/kg, q4 h to q8h (such as any of q4h, q5h, q6h, q7h, or q8h), for a maximum of 6 doses (such as 1, 2, 3, 4, 5, or 6 doses). In some embodiments, there is provided a method of treating hemophilia (e.g., hemophilia A or B, with or without inhibitor) in an individual (e.g., human), comprising administering to the individual an effective amount of a pharmaceutical composition; wherein the pharmaceutical composition comprises i) RVV-X in an amount of about 10 U/mL, ii) sucrose in an amount of about 30 mg/ml, iii) histidine in an amount of about 3 mg/ml, iv) polysorbate 20 in an amount of about 0.02% (w/v), and v) mannitol in an amount of about 40 mg/ml, wherein the pharmaceutical composition has a pH of about 6.85; and wherein the pharmaceutical composition is administered in a dose of about 0.1 U/kg or 0.16 U/kg q4 h for a maximum of 6 doses (such as 1, 2, 3, 4, 5, or 6 doses). In some embodiments, there is provided a method of treating hemophilia (e.g., hemophilia A or B, with or without inhibitor) in an individual (e.g., human), comprising administering to the individual an effective amount of a pharmaceutical composition; wherein the pharmaceutical composition comprises i) RVV-X in an amount of about 10 U/mL, ii) sucrose in an amount of about 30 mg/ml, iii) histidine in an amount of about 3 mg/ml, iv) polysorbate 20 in an amount of about 0.02% (w/v), and v) mannitol in an amount of about 40 mg/ml, wherein the pharmaceutical composition has a pH of about 6.85; and wherein the pharmaceutical composition is administered in a dose of about 0.1 U/kg or 0.16 U/kg q4 h for 4 doses. In some embodiments, the pharmaceutical composition is lyophilized. In some embodiments, the pharmaceutical composition is sterile. In some embodiments, the method further comprises reconstituting the pharmaceutical composition (e.g., with 0.9% sodium chloride injection) before administration. In some embodiments, the pharmaceutical composition is administered intravenously, such as by intravenous injection. In some embodiments, the RVV-X is isolated from Daboia russellii siamensis venom. In some embodiments, the purity of the RVV-X is at least about 95%. In some embodiments, the RVV-X comprises a) i) a heavy chain comprising the sequence of SEQ ID NO: 1; ii) a light chain 1 comprising the sequence of SEQ ID NO: 2; and iii) a light chain 2 comprising the sequence of SEQ ID NO: 3; b) i) a heavy chain comprising the sequence of SEQ ID NO: 1; ii) a light chain 1 comprising the sequence of SEQ ID NO: 5; and iii) a light chain 2 comprising the sequence of SEQ ID NO: 3; or c) a mixture of a) and b).

In some embodiments, the method of treating a bleeding disorder (e.g., hemophilia, such as hemophilia A or B, with or without inhibitor; or surgical wound bleeding) has one or more of the following biological activities: (i) activating FX and/or promoting FXa generation; (ii) increasing thrombin generation (TG) and/or endogenous thrombin-generating potential (ETP); (iii) shortening activated partial thromboplastin time (APTT), prothrombin time (PT), and/or thrombin time (TT); (iv) promoting hemostasis, improving bleeding signs, such as reducing bleeding time and/or amount, or stopping bleeding; (v) reducing mortality; (vi) promoting wound healing; (vii) prolonging patient survival; and/or (viii) reducing or relieving pain; etc., compared to an individual of bleeding disorder not receiving the described treatment or receiving a control agent (e.g., placebo or other hemostatic agents). In some embodiments, the method of activating FX and/or promoting FXa generation can increase FXa for at least about any of 50%, 60%, 70%, 80%, 90%, 100%, 1.5-fold, 2-fold, 5-fold, 10-fold, 30-fold, or more. In some embodiments, the method of increasing TG and/or ETP can increase TG and/or ETP for at least about any of 50%, 60%, 70%, 80%, 90%, 100%, 1.5-fold, 2-fold, 5-fold, 10-fold, 30-fold, or more. In some embodiments, the method of shortening APTT, PT, and/or TT can shorten APTT, PT, and/or TT for at least about any of 10%, 20%, 30%, 40%, 50%, 60%, 70%, 80%, 90%, 95%, or more. In some embodiments, the method of promoting hemostasis can reduce bleeding time and/or amount for at least about any of 10%, 20%, 30%, 40%, 50%, 60%, 70%, 80%, 90%, 95%, or more. In some embodiments, the method of reducing mortality can reduce mortality at least about any of 30%, 40%, 50%, 60%, 70%, 80%, 90%, 95%, or 100%. In some embodiments, the method of promoting wound healing can promote at least about 1.1 folds (including for example at least about any of 1.2, 1.3, 1.4, 1.5, 1.6, 1.7, 1.8, 1.9, 2, 3, 4, 5, 6, 7, 8, 9, 10, 20, 30, 40, or 50 folds, or more) of wound healing. In some embodiments, the method of prolonging survival of an individual (e.g., human) can prolong the survival of the individual by at least any of 1, 2, 3, 4, 5, 6, 7, 8, 9, 10, 11, 12, 18, 24 months, or 2, 3, 4, 5, 6, 7, 8, 9, 10 years, or longer. In some embodiments, the method of reducing or relieving pain can reduce pain for at least about any of 10%, 20%, 30%, 40%, 50%, 60%, 70%, 80%, 90%, 95%, or 100%.

Administration of the FX activator (e.g., RVV-X) or pharmaceutical composition thereof (e.g., RVV-X lead formulation) described herein may be carried out in any convenient manner, including by injection or transfusion. The route of administration is in accordance with known and accepted methods, such as by single or multiple bolus or infusion over a long period of time in a suitable manner. The FX activator or pharmaceutical composition thereof may be administered to a patient parentally (e.g., subcutaneously, intravenously, or intraperitoneally). In some embodiments, the FX activator or pharmaceutical composition thereof is administered systemically. In some embodiments, the FX activator or pharmaceutical composition thereof is administered to an individual by infusion, such as intravenous infusion. Infusion techniques for immunotherapy are known in the art (see, e.g., Rosenberg el al., New Eng. J. of Med. 319: 1676 (1988)). In some embodiments, the FX activator or pharmaceutical composition thereof is administered to an individual by intradermal or subcutaneous (i.e. beneath the skin) injection. For subcutaneous or intravenous injection, the FX activator or pharmaceutical composition thereof may be injected using a syringe. However, other devices for administration are available such as injection devices; injector pens; auto-injector devices, needleless devices; and subcutaneous patch delivery systems. In some embodiments, the FX activator or pharmaceutical composition thereof is administered by intravenous injection. In some embodiments, the FX activator or pharmaceutical composition thereof is administered locally to a site of damage or injury, such as directly to wound tissue or bleeding site. In some embodiments, the FX activator or pharmaceutical composition thereof is administered by sustained release or extended-release means.

In any of the examples herein of methods for treating a bleeding disorder, the method also can include administering one or more additional coagulation factors. For example, the one or more additional coagulation factors can be plasma purified or recombinant coagulation factors, procoagulants, such as vitamin K, vitamin K derivative and protein C inhibitors, plasma, platelets, red blood cells or corticosteroids.

### Bleeding Disorders

As used herein, “disease” or “disorder” refers to a pathological condition in an organism resulting from cause or condition including, but not limited to, infections, acquired conditions, genetic conditions, and characterized by identifiable symptoms. Diseases and disorders of interest herein are those involving coagulation, including those mediated by coagulation proteins and those in which coagulation proteins play a role in the etiology or pathology. Diseases and disorders also include those that are caused by the absence of a protein such as in hemophilia, and of particular interest herein are those disorders where coagulation does not occur due to a deficiency of defect in a coagulation protein. Exemplary diseases and disorders, such as, but not limited to, blood coagulation disorders, hematologic disorders, hemorrhagic disorders, hemophilias, coagulation factor deficiencies, and acquired blood disorders including bleeding associated with trauma and surgery.

As used herein, “bleeding disorder” refers to a condition in which the subject has a decreased ability to control bleeding due to poor blood clotting. Bleeding disorders can be inherited or acquired, and can result from, for example, defects or deficiencies in the coagulation pathway, defects or deficiencies in platelet activity, or vascular defects. In some embodiments, the bleeding disorder is a congenital bleeding disorder or an acquired bleeding disorder.

As used herein, “acquired bleeding disorder” refers to bleeding disorders that results from clotting deficiencies caused by conditions such as liver disease, vitamin K deficiency, or coumadin (warfarin) or other anti-coagulant therapy.

The bleeding disorder can be a disorder due to a deficiency of a coagulation factor, a disorder due to the presence of acquired inhibitors to a coagulation factor, a hematologic disorder, a hemorrhagic disorder, Von Willebrands' disease, a disorder that results from anticoagulant therapy with a vitamin-K antagonist, hereditary platelet disorders, vitamin K epoxide reductase Cl deficiency, gamma-carboxylase deficiency, bleeding associated with trauma, injury, thrombosis, thrombocytopenia, stoke, coagulopathy, disseminated intravascular coagulation (DIC), Bernard Soulier syndrome, Glanzman thromblastemia, or storage pool deficiency. In examples where the bleeding disorder is due to a deficiency of a coagulation factor or due to the presence of acquired inhibitors to a coagulation factor, the coagulation factor can be factor VII, factor IX, factor X, factor XI, factor V, factor XII, factor II, or von Willebrand factor. In examples where the bleeding disorder is due to a deficiency of a coagulation factor, the coagulation factor can be factor VII, factor VIII, factor IX, factor XI. In some embodiments, the bleeding disorder is due to a deficiency of a coagulation factor. In some embodiments, the bleeding disorder is hemophilia. For example, the bleeding disorder can be hemophilia A, hemophilia B or hemophilia C. In some embodiments, the hemophilia is hemophilia with inhibitors (e.g., antibody). In some embodiments, the bleeding disorder is hemophilia A without inhibitor. In some embodiments, the bleeding disorder is hemophilia A with FVIII inhibitor (antibody against FVIII, such as exogenous FVIII). In some embodiments, the bleeding disorder is hemophilia B without inhibitor. In some embodiments, the bleeding disorder is hemophilia B with FIX inhibitor (antibody against FIX, such as exogenous FIX).

In examples of methods herein where the bleeding disorder is due to a deficiency of a coagulation factor, the disorder is a familial multiple coagulation factor deficiency (FMFD). In examples herein where the bleeding disorder is due to the presence of acquired inhibitors to a coagulation factor, the coagulation factor is factor VII, factor VIII, factor IX, factor X, factor XI, and factor XII. In such examples, the acquired inhibitors are autoantibodies. For example, the bleeding disorder is acquired hemophilia. In examples of methods herein where the bleeding disorder is a hereditary platelet disorder it can be Chediak-Higashi syndrome, Hermansky-Pudlak syndromes, thromboxane A2 dysfunction, Glanzmann's thrombasthenia, and Bernard-Soulier syndrome. In other examples herein, where the disorder results from anticoagulant therapy with a vitamin-K antagonist, the vitamin-K antagonist can be heparin, pentasaccharide, warfarin, small molecule antithrombotics and FXa inhibitors.

In other examples of methods herein, the disorder is thrombosis, thrombocytopenia, stroke and coagulopathy. In further examples of methods herein the bleeding disorder results from a trauma, surgery, or wound. In such examples, for example, the bleeding is manifested as acute haemarthroses, chronic haemophilic arthropathy, haematomas, haematuria, central nervous system bleedings, gastrointestinal bleedings, or cerebral haemorrhage. In other examples, the bleeding is due to dental extraction. In other examples herein, where the bleeding disorder is due to surgery, the surgery is heart surgery, angioplasty, lung surgery, abdominal surgery, spinal surgery, brain surgery, vascular surgery, dental surgery, or organ transplant surgery. For example, the surgery is transplant surgery is by transplantation of bone marrow, heart, lung, pancreas, or liver.

### Congenital Bleeding Disorders

FX activator (e.g., RVV-X) or pharmaceutical composition thereof (e.g., RVV-X lead formulation) described herein can be used to bypass any coagulation factor in either the intrinsic or extrinsic coagulation pathways, and treat coagulopathy due to the deficiencies thereof. Bleeding disorders resulting from congenital coagulation factor deficiency, include hemophilia A (Factor VIII deficiency); hemophilia B (Factor IX deficiency); hemophilia C (Factor XI deficiency); Factor VII deficiency; Factor X deficiency; Factor XII deficiency; and types I, II, IV, V, and VI familial multiple coagulation factor deficiencies (FMFD) (see Roberts, H R and M D Bingham, “Other Coagulation Factor Deficiencies,” Thrombosis and Hemorrhage, 2nd ed. Baltimore, Md.: Williams & Wilkins, 1998: 773-802). FX activator or pharmaceutical composition thereof also can be used in the treatment of additional congenital bleeding diseases and disorders, such as, but not limited to, Von Willebrand's disease, hereditary platelet disorders (e.g., storage pool disease such as Chediak-Higashi and Hermansky-Pudlak syndromes, thromboxane A2 dysfunction, Glanzmann's thrombasthenia, and Bernard-Soulier syndrome), and Hereditary Hemorrhagic Telangiectsasia, also known as Rendu-Osler-Weber syndrome.

Other congenital bleeding disorders can be treated with the FX activators (e.g., RVV-X) or pharmaceutical compositions thereof (e.g., RVV-X lead formulation) described herein to promote coagulation. Spontaneous and surgery associated bleeding episodes associated with von Willebrand disease (vWD) can be treated using the FX activator or pharmaceutical composition thereof. vWD is a bleeding disorder caused by a defect or deficiency of the blood clotting protein, von Willebrand Factor (vWF), and is estimated to occur in 1% to 2% of the population. Subjects with vWD bruise easily, have recurrent nosebleeds, bleed after tooth extraction, tonsillectomy or other surgery, and women patients can have increased menstrual bleeding. FX activators or pharmaceutical compositions thereof can be used to ameliorate spontaneous and surgery-associated bleeding in vWD patients.

Other platelet-related bleeding disorders, such as for example, Glanzmann's thrombasthenia and Hermansky-Pudlak syndrome also are associated with reduced endogenous clotting activity. Excess spontaneous or surgery-associated bleeding in patients with platelet related bleeding disorders also can be controlled by therapeutic doses of the FX activators (e.g., RVV-X) or pharmaceutical compositions thereof (e.g., RVV-X lead formulation) described herein. For example, a patient with Glanzmann's thrombasthenia undergoing surgery can be treated before, during and/or after surgery with the FX activators or pharmaceutical compositions thereof to prevent major blood loss.

### Acquired Bleeding Disorders

Bleeding disorders also can be acquired, rather than congenital. FX activators (e.g., RVV-X) or pharmaceutical compositions thereof (e.g., RVV-X lead formulation) described herein also can be used to treat acquired bleeding disorders. Acquired bleeding disorders include coagulopathy as a result of surgical procedures or drug-induced coagulopathy, for example thrombocytopenia due to chemotherapeutic regimens. In some example, FX activators or pharmaceutical compositions thereof can be used as an antidote to an overdose of an anti-coagulant therapeutic. FX activators or pharmaceutical compositions thereof also can be used to treat acquired coagulation factor deficiencies, such as acquired factor X deficiency as a result of liver disease, vitamin K deficiency. Other acquired bleeding disorders that can benefit from FX activators or pharmaceutical compositions thereof treatment include hemolytic-uremic syndrome, allergic purpura (Henoch Schonlein purpura) and disseminated intravascular coagulation (DIC).

In some embodiments, the FX activators (e.g., RVV-X) or pharmaceutical compositions thereof (e.g., RVV-X lead formulation) described herein can be used to treat bleeding episodes due to trauma, or surgery, or lowered count or activity of platelets, in a subject. For example, hemodilutional coagulopathy, for example as a result of blood transfusion, or acute traumatic coagulopathy following trauma can be treated with FX activators or pharmaceutical compositions thereof. Exemplary methods for patients undergoing surgery include treatments to prevent hemorrhage and treatments before, during, or after surgeries such as, but not limited to, heart surgery, angioplasty, lung surgery, abdominal surgery, spinal surgery, brain surgery, vascular surgery, dental surgery, or organ transplant surgery, including transplantation of bone marrow, heart, lung, pancreas, or liver. In some embodiments, the acquired bleeding disorder is chemotherapy-acquired thrombocytopenia, other coagulopathies, transplant-acquired bleeding (e.g., severe bleeding following bone marrow transplant (BMT) or stem cell transplant (SCT)), anticoagulant therapy-induced bleeding (patients undergoing anticoagulant therapies for the treatment of conditions, such as thromboembolism, can exhibit bleeding episodes upon acute administration of anticoagulants, such as warfarin, heparin, fondaparinux, and Rivaroxaban, or develop hemorrhagic disorders as a result long term usage of such therapies), or acquired hemophilia.

### Trauma and Surgical Bleeding

FX activators (e.g., RVV-X) or pharmaceutical compositions thereof (e.g., RVV-X lead formulation) described herein can be used as therapy to treat bleeding associated with perioperative and traumatic blood loss in subjects with normal coagulation systems. For example, FX activators or pharmaceutical compositions thereof can be administered to a patient to promote coagulation and reduce blood loss associated with surgery and, further, reduce the requirement for blood transfusion.

In some examples, FX activators (e.g., RVV-X) or pharmaceutical compositions thereof (e.g., RVV-X lead formulation) described herein can be administered to patients with normal coagulation undergoing various types of surgery to effect rapid hemostasis and prevent blood loss. Treatment with FX activators or pharmaceutical compositions thereof can promote hemostasis at the site of surgery and reduce or prevent blood loss, thereby reducing or abolishing the need for transfusion. In some embodiments, FX activators or pharmaceutical compositions thereof can exhibit enhanced properties such as increased half-life, increased resistance to circulating protease inhibitors, and/or increased catalytic activity, and might therefore be administered, for example, at lower doses, less frequently, and with fewer adverse reactions.

FX activators (e.g., RVV-X) or pharmaceutical compositions thereof (e.g., RVV-X lead formulation) described herein also can be used to promote coagulation and prevent blood loss in subjects with traumatic injury. Trauma is defined as an injury to living tissue by an extrinsic agent, and is the fourth leading cause of death in the United States. Trauma is classified as either blunt trauma (resulting in internal compression, organ damage and internal hemorrhage) or penetrative trauma (a consequence of an agent penetrating the body and destroying tissue, vessel and organs, resulting in external hemorrhaging). Trauma can be caused by several events including, but not limited to, vehicle accidents (causing blunt and/or penetrative trauma), gunshot wounds (causing penetrative trauma), stabbing wounds (causing penetrative trauma), machinery accidents (causing penetrative and/or blunt trauma), and falls from significant heights (causing penetrative and/or blunt trauma). Uncontrolled hemorrhage as a result of trauma is responsible for most of the associated mortality.

Diffuse coagulopathy is a relatively common complication associated with trauma patients, occurring in as many as 25-36% of subjects. Coagulopathy can develop early after injury, resulting from a variety of factors such as dilution and consumption of coagulation factors and platelets, fibrinolysis, acidosis, and hypothermia. Conventional management involves replacement therapy by transfusion with fresh frozen plasma (FFP) platelets, RBC and/or cryoprecipitate, correcting acidosis, and treating hypothermia. These steps often are insufficient to stop the bleeding and prevent death.

### Hemophilia

As used herein, “hemophilia” refers to a bleeding disorder caused by a deficiency in a blood clotting factors. Hemophilia can be the result, for example, of absence, reduced expression, or reduced function of a clotting factor. The most common type of hemophilia is hemophilia A, which results from a deficiency in factor VIll. The second most common type of hemophilia is hemophilia B, which results from a deficiency in factor IX. Hemophilia C, also called FXI deficiency, is a milder and less common form of hemophilia.

As used herein, “congenital hemophilia” refers to types of hemophilia that are inherited. Congenital hemophilia results from mutation, deletion, insertion, or other modification of a clotting factor gene in which the production of the clotting factor is absent, reduced, or non-functional. For example, hereditary mutations in clotting factor genes, such as factor VIII and factor IX result in the congenital hemophilias, Hemophilia A and B, respectively.

As used herein, “acquired hemophilia” refers to a type of hemophilia that develops in adulthood from the production of autoantibodies that inactivate coagulation factor such as FVIII. Factor VIII inhibitors can develop spontaneously in otherwise healthy individuals, resulting in a condition known as “acquired hemophilia”. Acquired hemophilia is a rare condition, with a yearly incidence of 0.2-1.0 per million population. The autoantibodies are mainly IgG4 antibodies, which, when bound to FVIII, inhibit FVIII activity by interfering with thrombin cleavage, von Willebrand factor interaction and/or phospholipid binding. This results in life-threatening hemorrhage in approximately 87% of affected patients. Common sites of bleeding are skin, mucosa, muscles and retroperitoneum, in contrast to patients with hereditary hemophilia who bleed predominantly in joints and muscles. Acquired hemophilia can be treated with an activated prothrombin complex concentrate or recombinant activated factor VII (NovoSeven®, Novo Nordisk) to control bleeding episodes. The FX activators (e.g., RVV-X) or pharmaceutical compositions thereof (e.g., RVV-X lead formulation) described herein can exhibit enhanced coagulation activity and bypass the need for FVII replacement therapy.

Hemophilia is a bleeding disorder that is caused by a deficiency in one or more blood coagulation factors. It is characterized by a decreased ability to form blood clots at sites of tissue damage. Congenital X-linked hemophilias include hemophilia A and hemophilia B, which are caused by mutation(s) resulting in deficiencies in FVIII and FIX, respectively. Hemophilia A occurs at a rate of 1 out of 10,0000 males, while hemophilia B occurs in 1 out of 50,000 males. Hemophilia A and B are further classified as mild, moderate, or severe. A plasma level with 5%-25% of normally functioning factor VIII or IX is classified as mild, 1%-5% is moderate, and less than 1% is severe. Hemophilia C, often referred to as FXI deficiency, is a relatively mild and rare autosomal recessive disease, affecting about 1 in 100000 people.

Patients with hemophilia suffer from recurring joint and muscle bleeds, which can be spontaneous or in response to trauma. The bleeding can cause severe acute pain, restrict movement, and lead to secondary complications including synovial hypertrophy. Furthermore, the recurring bleeding in the joints can cause chronic synovitis, which can cause joint damage, destroying synovium, cartilage, and bone. Bleeding is generally treated with transfusion of fresh frozen plasma (FFP), FXI replacement therapy, or, for topical treatment, such treatment of external wounds or dental extractions, fibrin glue. The most common treatment for hemophilia A or B is replacement therapy, in which the patient is administered FVIII or FIX. The formulations are available commercially as plasma-derived or recombinant products, with recombinant proteins now being the treatment of choice in previously untreated patients. While these therapies can be very successful, complications arise if the patient develops inhibitors to the newly administered factor VIII or factor IX.

Inhibitors are IgG antibodies, mostly of the IgG4 subclass, that react with FVIII or FIX and interfere with pro-coagulant function. Inhibitors affect about 1 in 5 patients with severe hemophilia A. Most subjects develop these inhibitors soon after administration of the first infusions of factor VIII, which is often in early childhood, although subjects develop inhibitors later in life. Inhibitors also affect about 1 in 15 people with mild or moderate hemophilia A. These inhibitors usually develop during adulthood and not only destroy administered exogenous FVIII, but also destroy endogenous FVIII. As a result, mild and moderate hemophiliacs become severe. Clinically, hemophilia A patients with inhibitors are classified into high and low responders according to the strength of the anamnestic response they experience when they are re-exposed to FVIII. Inhibitors affect about 1 in 100 patients with hemophilia B. In most cases, the inhibitors develop after the first infusions of therapeutic factor IX and can be accompanied by allergic reactions.

The FX activators (e.g., RVV-X) or pharmaceutical compositions thereof (e.g., RVV-X lead formulation) described herein and the nucleic acids encoding the FX activators provided herein can be used universally in therapies for patients with hemophilia, including hemophilia patients with inhibitors, and can be used for the treatment of bleeding conditions associated with hemophilia. The FX activators or pharmaceutical compositions thereof described herein can be used, for example, to control or prevent spontaneous bleeding episodes or to control or prevent bleeding in response to trauma or surgical procedures, by enhancing thrombin generation while bypassing the requirement for FVIIIa and/or FIXa.

The FX activators (e.g., RVV-X) or pharmaceutical compositions thereof (e.g., RVV-X lead formulation) described herein can be tested for therapeutic effectiveness, for example, by using animal models. For example, FVIII or FIX-deficient mice (e.g., M-KOFVIII mice), antibody-induced hemophilic mice, or any other known disease model for hemophilia, can be treated with FX activators or pharmaceutical compositions thereof. Progression of disease symptoms and phenotypes is monitored to assess the effects of the FX activators or pharmaceutical compositions thereof. FX activators or pharmaceutical compositions thereof also can be administered to animal models as well as to subjects, such as in clinical trials, to assess in vivo effectiveness in comparison to placebo controls and/or controls using other hemostatic agents.

The FX activators (e.g., RVV-X) or pharmaceutical compositions thereof (e.g., RVV-X lead formulation) described herein also can be used to treat patients with coagulopathy resulting from deficiencies in coagulation factors other than FVIII and FIX. For example, FX activators or pharmaceutical compositions thereof can be used to treat patients with Factor VII deficiency, Factor X deficiency, Factor XIII deficiency, and types II, IV, V, and VI familial multiple coagulation factor deficiencies (FMFD) (see Roberts, H R and M D Bingham, “Other Coagulation Factor Deficiencies,” Thrombosis and Hemorrhage, 2nd ed. Baltimore, Md.: Williams & Wilkins, 1998: 773-802).

### Pharmacokinetics (PK)

Pharmacokinetics (PK) refers to the absorption, distribution, metabolism, and excretion of a drug (e.g., FX activator such as RVV-X) once it has been administered to a subject. Pharmacokinetic parameters that may be useful in determining clinical utility include but are not limited to serum/plasma concentration, serum/plasma concentration over time, maximum serum/plasma concentration (Cₘₐₓ), time to reach maximum concentration (Tₘₐₓ), half-life (t_{1/2}), area under concentration time curve within the dosing interval (AUC), etc.

Techniques for obtaining a PK curve of a drug are known in the art. See, e.g., Heller et al., Annu Rev Anal Chem, 11, 2018; and Ghandforoush-Sattari et al., J Amino Acids, Article ID 346237, Volume 2010. In some embodiments, the PK curve of the FX activator (e.g., RVV-X) in an individual is measured in a blood, plasma, or serum sample from the individual. In some embodiments, the PK curve of the FX activator (e.g., RVV-X) in an individual is measured using a mass spectrometry (MS) technique, such as LC-MS/MS, or ELISA. PK analysis on PK curves can be conducted by any methods known in the art, such as non-compartmental analysis, e.g., using PKSolver V2 software (Zhang Y. el al., “PKSolver: An add-in program for pharmacokinetic and pharmacodynamic data analysis in Microsoft Excel,” Comput Methods Programs Biomed. 2010; 99(3):306-1). Also see Example 1.

“C” denotes the concentration of drug in blood plasma, serum, or in any appropriate body fluid or tissue of a subject, and is generally expressed as mass per unit volume, for example nanograms or pigograms per milliliter. For convenience, the concentration of drug in serum or plasma is referred to herein as “serum concentration” or “plasma concentration.” The serum/plasma concentration at any time following drug administration (e.g., FX activator, such as i.v. administration) is referenced as Cₜᵢₘₑ or Cₜ. The maximum serum/plasma drug concentration during the dosing period is referenced as Cₘₐₓ, while Cₘᵢₙ refers to the minimum serum/plasma drug concentration at the end of a dosing interval; and Cₐᵥₑ refers to an average concentration during the dosing interval.

The term “bioavailability” refers to an extent to which—and sometimes rate at which—the drug (e.g., FX activator such as RVV-X) enters systemic circulation, thereby gaining access to the site of action.

“AUC” is the area under the serum/plasma concentration-time curve and is considered to be the most reliable measure of bioavailability, such as area under concentration time curve within the dosing interval (AUC_{τ}), “overall exposure” or “total drug exposure across time” (AUC₀₋ₗₐₛₜ or AUC_{0-inf}), area under concentration time curve at time t post-administration (AUC₀₋ₜ), etc.

Serum/plasma concentration peak time (Tₘₐₓ) is the time when peak serum/plasma concentration (Cₘₐₓ) is reached after administration of a drug.

Half-life (t_{1/2}) is the amount of time required for the drug (e.g., FX activator such as RVV-X) concentration measured in plasma or serum (or other biological matrices) to be reduced to exactly half of its concentration or amount at certain time point. For example, after IV dosing, the drug concentrations in plasma or serum decline due to both distribution and elimination. In a plasma or serum profile of drug concentration over time post-IV doing, the first phase or rapid decline is considered to be primarily due to distribution, while the later phase of decline is usually slower and considered to be primarily due to elimination, although both processes occur in both phases. Distribution is assumed to be complete after sufficient time. In general, the elimination half-life is determined from the terminal or elimination (dominant) phase of the plasma/serum concentration versus time curve. See, e.g., Michael Schrag and Kelly Regal, “Chapter 3—Pharmacokinetics and Toxicokinetics” of “A Comprehensive Guide to Toxicology in Preclinical Drug Development”, 2013.

In some embodiments, the FX activator (e.g., RVV-X) or pharmaceutical composition thereof (e.g., RVV-X lead formulation) described herein has a half-life (e.g., i.v. administration, such as to human) of at least about 5 hours, such as at least about any of 5.5, 6, 6.5, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.7, 9, 9.5, 10, 10.5, 11, 12, 13, 14, 15, 16, 17, 18, 19, 20, 21, 22, 23, 24, 36, 48 hours, or longer. In some embodiments, the FX activator or pharmaceutical composition thereof has a half-life (e.g., i.v. administration, such as to human) of from about 5 hrs to about 12 hrs, such as any of from about 5 hrs to about 10 hrs, from about 5 hrs to about 8.5 hrs, from about 6.5 hrs to about 9 hrs, from about 6.7 hrs to about 8.8 hrs, or from about 7 hrs to about 8.5 hrs. In some embodiments, the FX activator or pharmaceutical composition thereof is administered in a single administration, such as a single intravenous injection, such as in a dose of from about 0.1 U/kg to about 0.48 U/kg or about 0.16 U/kg to about 0.48 U/kg. In some embodiments, the FX activator or pharmaceutical composition thereof is administered in multiple doses (e.g., 4-6 doses, such as 5 doses), such as intravenous injection in about 0.1 U/kg or 0.16 U/kg per dose, q4h.

In some embodiments, a single or multiple (e.g., 4-6 doses, such as 5 doses, q4h) administration (e.g., i.v. administration, such as to human) of the FX activator (e.g., RVV-X) or pharmaceutical composition thereof (e.g., RVV-X lead formulation) described herein can provide an AUC_{0-∞} of from about 600 h*pg/mL to about 4000 h*pg/mL, such as any of from about 650 h*pg/mL to about 3500 h*pg/mL, from about 650 h*pg/mL to about 1500 h*pg/mL, from about 900 h*pg/mL to about 2000 h*pg/mL, from about 1500 h*pg/mL to about 3500 h*pg/mL, from about 3200 h*pg/mL to about 3500 h*pg/mL, from about 1200 h*pg/mL to about 1500 h*pg/mL, from about 800 h*pg/mL to about 1000 h*pg/mL, from about 2200 h*pg/mL to about 2600 h*pg/mL, from about 1000 h*pg/mL to about 4000 h*pg/mL, from about 2000 h*pg/mL to about 4000 h*pg/mL, from about 3000 h*pg/mL to about 4000 h*pg/mL, or from about 600 h*pg/mL to about 1000 h*pg/mL. In some embodiments, the FX activator or pharmaceutical composition thereof is administered from about 0.1 U/kg to about 0.48 U/kg or about 0.16 U/kg to about 0.48 U/kg per dose (or as a single dose), such as about 0.1 U/kg or 0.16 U/kg per dose.

In some embodiments, a single administration (e.g., i.v. administration, such as to human) of the FX activator (e.g., RVV-X) or pharmaceutical composition thereof (e.g., RVV-X lead formulation) described herein, such as in a dose of from about 0.1 U/kg to about 0.48 U/kg or about 0.16 U/kg to about 0.48 U/kg, provides a Cₘₐₓ of from about 60 μg/mL to about 500 μg/mL, such as any of from about 70 μg/mL to about 450 μg/mL, from about 70 μg/mL to about 150 μg/mL, from about 100 μg/mL to about 500 μg/mL, from about 200 μg/mL to about 500 μg/mL, from about 300 μg/mL to about 500 μg/mL, from about 400 μg/mL to about 500 μg/mL, from about 100 μg/mL to about 300 μg/mL, from about 90 μg/mL to about 120 μg/mL, from about 100 μg/mL to about 250 μg/mL, from about 140 μg/mL to about 180 μg/mL, from about 150 μg/mL to about 450 μg/mL, or from about 270 μg/mL to about 320 μg/mL. In some embodiments, Cₘₐₓ is achieved within at most about 10 minutes (e.g., at most about any of 9, 8, 7, 6, 5, 4, 3, 2.1 minute) after administration, such as about 5 minutes after administration.

### Pharmacodynamics (PD)

FX activators (e.g., RVV-X) or pharmaceutical compositions thereof (e.g., RVV-X lead formulation) described herein can be evaluated for biological activity using assays or in vivo parameters, such as in vitro coagulation assays or a pharmacodynamic (PD) effect in a hemophilia model or individual.

PD parameters or assays for testing coagulation activity are well known in the art, including but not limited to, Activated Partial Thromboplastin Time (APTT), factor X activity assay (FX:C), thrombin generation (TG) including the peak height, calibrated automated thrombin generation assay (CAT), endogenous thrombin-generating potential (ETP), Activated Clotting Time (ACT), Whole Blood Clotting Time (WBCT), etc. The APTT test is used to measure and evaluate all the clotting factors of the intrinsic and common pathways of the clotting cascade by measuring the time (in seconds) it takes a clot to form after adding a drug to a plasma sample. APTT can be assessed using the APTT-ACTIN FSL kit (Siemens, Germany), the CS-5100 (Sysmex, Japan) automated blood coagulation analyzer, or the STA-CK Prest (Stago, France) and the STA-R Max (Stago) automated blood coagulation analyzer. FX:C can be assessed by the validated coagulation assay using the automated blood coagulation analyzer (Sysmex), or FX Chromogenic Activity Assay Kit. TG can be measured by the calibrated automated thrombogram assay using the TG (CAT) analyzer (Stago), such as with tissue factor (TF) and phospholipid (Stago) as triggers. Also see Example 1.

In some embodiments, a single administration (e.g., i.v. administration, such as to human) of the FX activator (e.g., RVV-X) or pharmaceutical composition thereof (e.g., RVV-X lead formulation) described herein, such as in a dose of from about 0.01 U/kg to about 0.48 U/kg, can i) increase TG's peak height and ETP in a dose-dependent manner; ii) reach TG and/or ETP peak at about 5 minutes; iii) remain TG and/or ETP at a high level until about 4 hours after administration, and/or iv) return TG and/or ETP to pre-dose level after about 24 hours.

In some embodiments, a single administration (e.g., i.v. administration, such as to human) of the FX activator (e.g., RVV-X) or pharmaceutical composition thereof (e.g., RVV-X lead formulation) described herein, such as in a dose of from about 0.01 U/kg to about 0.48 U/kg, can i) shorten APTT in a dose-dependent manner; ii) shorten APTT for at least about 5% (e.g., at least about any of 10%, 15%, 20%, 25%, 30%, 35%, 40%, 45%, 50%, 60%, 70%, or more); iii) reach maximum APTT shortening at about 5 minutes post-administration; and/or iv) return APTT to pre-dose level after about 24 hours.

In some embodiments, multiple administrations (e.g., i.v. administration, such as to human) of the FX activator (e.g., RVV-X) or pharmaceutical composition thereof (e.g., RVV-X lead formulation) described herein, such as in a dose of about 0.1 U/kg or 0.16 U/kg q4 h for 2-6 (e.g., 2, 3, 4, 5, or 6) doses, can i) shorten APTT within about 10 minutes after the first administration; ii) shorten APTT for at least about 20% (e.g., at least about any of 25%, 30%, 35%, 40%, 45%, 50%, 60%, 70%, or more); iii) shorten APTT at least about 20 seconds (e.g., at least about any of 21, 22, 23, 24, 25, 26, 27, 28, 29, 30, 31, 32, 33, 34, 35, 40, 50, 60, 70, 80 seconds, or more); iv) reach APTT plateau about 4 hours after the third administration; v) shorten APTT for about 8 hours after the last administration; and/or vi) return APTT to pre-dose level at about 28 hours to about 36 hours after the last administration.

### Safety

Safety outcomes of administering FX activator (e.g., RVV-X) or pharmaceutical composition thereof (e.g., RVV-X lead formulation) described herein can be assessed by vital signs, physical examination, laboratory test results, electrocardiogram (ECG), ultrasound (abdominal and deep veins of both lower limbs), and adverse events profile. The severity of adverse events can be determined with reference to the NCI Common Terminology Criteria for Adverse Events (CTCAE v5.0). Also see Example 1, Table 4-1, Table 4-2, Table Si, Table S2 for safety measurement methods and parameters. For example, dose-limiting toxicity (DLT) event, serious adverse events (SAE), adverse event (AE) that leads to study withdrawal, dose-dependent AE, drug-related AE, drug-related SAE, or Suspected Unexpected Serious Adverse Reaction (SUSAR) can be examined. Other adverse parameters include, but are not limited to, clinically relevant coagulation abnormalities, lower extremity deep venous thrombosis (DVT), thromboembolic events, increase of D-dimer (protein fragment generated when a blood clot dissolves), increase of fibrin degradation product (FDP), etc. Adverse parameters also include FX level and/or fibrinogen (FIB) level decreasing beyond the range of normal values, and/or not returning to normal range after a period of time (e.g., 3 hours).

In some embodiments, the methods of treating bleeding disorders described herein (e.g., single or multiple administration), and FX activators (e.g., RVV-X) or pharmaceutical composition thereof (e.g., RVV-X lead formulation) described herein, induce no or low adverse activity, such as measured by one or more of the parameters and/or assays described herein. No or low adverse activity include, but are not limited to, AE (e.g., drug-related AE) are within grades 1-2; grade 3 AE is less than about 5% (e.g., less than about any of 4%, 3%, 2%, 1%, or 0%) of total AE; each of dose-dependent AE, DLT event, SAE, AE that leads to study withdrawal, drug-related SAE, SUSAR, clinically relevant coagulation abnormalities, DVT, DIC, increase of D-dimer, increase of FDP, and/or thromboembolic events, is less than about 5% (e.g., less than about any of 4%, 3%, 2%, 1%, or 0%) of total AE: the decrease of FX level and/or FIB level is within the range of normal values; etc.

### Immunogenicity

The term “immunogenicity”, in the context of administering a protein drug to a patient, is defined as the propensity of that protein drug to illicit an immune response in the patient after dosing, or after repeat dosing.

Anti-FX activator (e.g., anti-RVV-X) binding antibody in plasma can be examined by a validated electrochemiluminescence assay (Meso Scale Discovery). Antibody titer and neutralizing antibody (against FX activator) can be further assessed.

In some embodiments, the methods of treating bleeding disorders described herein (e.g., single or multiple administration), and FX activators (e.g., RVV-X) or pharmaceutical composition thereof (e.g., RVV-X lead formulation) described herein, do not induce neutralizing antibody, or induce low titer (e.g., less than about any of 30, 20, 10, 5, or 2) of neutralizing antibody.

In some embodiments, the methods of treating bleeding disorders described herein (e.g., single or multiple administration), and FX activators (e.g., RVV-X) or pharmaceutical composition thereof (e.g., RVV-X lead formulation) described herein, i) do not induce anti-FX activator (e.g., anti-RVV-X) binding antibody in plasma, or induce low titer (e.g., less than 30) of binding antibody; and/or ii) do not induce increasement of the inhibitor titer, or induce within about 10% increasement of the inhibitor titer.

### V. Articles of Manufacture and Kits

Further provided are kits, unit dosages, and articles of manufacture comprising any of the FX activators (e.g., RVV-X) or pharmaceutical compositions thereof (e.g., RVV-X lead formulation) described herein. In some embodiments, a kit is provided which contains any one of the FX activator pharmaceutical compositions described herein and preferably provides instructions for its use, such as for use in the treatment of bleeding disorders described herein (e.g., hemophilia A or B, with or without inhibitor).

In some embodiments, the FX activator (e.g., RVV-X) pharmaceutical composition (e.g., lyophilized; such as RVV-X lead formulation) is contained in a vial (e.g., having a stopper pierceable by a hypodermic injection needle). In some embodiments, the pharmaceutical composition is in powder form. In some embodiments, each vial contains about 5 U of the FX activator (e.g., RVV-X). In some embodiments, the kit further provides an instruction that the pharmaceutical composition is for reconstitution with 2 ml of 0.9% sodium chloride injection solution. In some embodiments, the kit further provides an instruction that the pharmaceutical composition is for intravenous administration. In some embodiments, the kit further comprises an injection needle. In some embodiments, the kit further comprises 0.9% sodium chloride injection solution in a separate container.

Kits of the invention include one or more containers comprising an FX activator (e.g., RVV-X) or pharmaceutical composition thereof (e.g., RVV-X lead formulation) described herein, e.g., for treating a bleeding disorder. For example, the instructions comprise a description of administration of the FX activator (e.g., RVV-X) or pharmaceutical composition thereof to treat a bleeding disorder, such as hemophilia. The kit may further comprise a description of selecting an individual (e.g., human) suitable for treatment based on identifying whether that individual has the disease and the type of the disease. The instructions relating to the use of the FX activator (e.g., RVV-X) or pharmaceutical composition thereof generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable. The kits of the present application are in suitable packaging. Suitable packaging includes, but is not limited to, vials, bottles, jars, flexible packaging (e.g., sealed Mylar or plastic bags), and the like. Also contemplated are packages for use in combination with a specific device, such as an injection device such as an injection needle. A kit may have a sterile access port (for example, the container may be an intravenous solution bag or a vial having a stopper pierceable by a hypodermic injection needle). At least one active agent in the composition is an FX activator (e.g., RVV-X) as described herein. The container may further comprise a second pharmaceutically active agent. The kits may optionally provide additional components such as buffers and interpretive information. Normally, the kit comprises a container and a label or package insert(s) on or associated with the container.

The present application also provides articles of manufacture, which include vials (such as sealed vials), bottles, jars, flexible packaging, and the like. The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder described herein, and may have a sterile access port (for example, the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g. from 2-6 administrations) of the reconstituted formulation. Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical composition and instructions for use, packaged in quantities sufficient for storage and use in pharmacies, for example, hospital pharmacies and compounding pharmacies.

### EXAMPLES

The examples below are intended to be purely exemplary of the invention and should therefore not be considered to limit the invention in any way. The following examples and detailed description are offered by way of illustration and not by way of limitation.

### Example 1: a First-In-Human Phase I Study of FX Activator RVV-X, in Hemophilia A or B Patients with Inhibitor

Hemophilia A (HA) and hemophilia B (HB) are inherited bleeding disorders caused by deficiency of factor VIII (FVIII) or factor IX (FIX) respectively. Bleeding episodes (BEs) in these patients are usually treated with factor replacement therapy; however, 20%-30% of patients with hemophilia A, and 5% with hemophilia B, will develop inhibitors to exogenous FVIII or FIX¹. Bleeding episodes in these patients with inhibitor are difficult to control. In these patients, hemostasis may not be achievable with replacement of the deficient factor (depending upon the inhibitor titer) and thus may require administration of bypassing agents. The two currently recommended first-line by-pass agents for inhibitor patients are activated thrombin complex concentrates (aPCC) and activated recombinant factor VII (rFVIIa)²⁻⁴. However, aPCC is not available in China and trace amounts of factor VIII in aPCC may induce an anamnestic response to FVIII. rFVIIa is not widely used because of high medical expense especially in developing country.

RVV-X, purified from the venom of Daboia russellii siamensis, is a heterotrimer with a molecular weight of 93 kDa. It is composed of an a chain (heavy chain), a R chain (light chain 1) and a γ chain (light chain 2)⁸⁻¹⁰. RVV-X can specifically activate coagulation factor X (FX). This activation process includes cleaving FX at the specific “Arg-Ile” peptide bond in the N-terminal region of the FX heavy chain, so that the active site is fully exposed to produce coagulation factor Xa (FXa). FXa then forms prothrombin complex with platelets, coagulation factor Va (FVa) and calcium ions activated at the injury site, thus to increase the production of thrombin and achieve the purpose of hemostasis¹⁰. Preclinical studies have confirmed that RVV-X can activate FX to FXa, and increase the content of thrombin in human plasma and the peak value of thrombin production in a dose-dependent manner. In addition, RVV-X can shorten the APTT of hemophilia mice, and reduce the 24-hour mortality of tail broken hemophilia mice. Preclinical toxicology studies have shown that RVV-X is well tolerated and safe.

Here, we present the phase I, first-in-human, multi-center, open-label, dose-escalation study of RVV-X, which evaluated the safety, pharmacokinetics (PK) and pharmacodynamics (PD) of RVV-X in hemophilia A or B patients with inhibitors to Factor VIII/IX.

Chinese HA or HB patients with inhibitors received a single intravenous injection of RVV-X (0.01 U/kg, 0.04 U/kg, 0.08 U/kg, 0.16 U/kg, 0.32 U/kg and 0.48 U/kg) in single-dose part or at most six injections of RVV-X (0.16 U/kg) every four hours (q4h) in multiple-dose part in non-bleeding state. This study was registered at www.clinicaltrials.gov with trial number as NCT-04747964 and NCT-05027230. Furthermore, a multiple-dose of 0.1 U/kg every four hours (q4h) for 6 injection was also evaluated.

Single-dose part results: RVV-X exhibited a linear PK profile. After RVV-X administration, both the change of thrombin generation (TG) peak height and endogenous thrombin-generating potential (ETP) increased in a dose-dependent manner. Activated partial thromboplastin time (APTT) was shortened in a dose-dependent manner.

Multiple-dose part results for 0.16 U/kg: After the first three administration of RVV-X, the increase of TG peak height and the shortening of APTT reached the plateau stage. Multiple-dose part results for 0.1 U/kg: After the first three administration of RVV-X, the shortening of APTT reached the plateau stage. The TG peak increased continuously during the administration.

To summarize, both single injection and multiple injection of RVV-X were safe and well tolerated. Starting from the single dose of 0.16 U/kg, RVV-X significantly improved the coagulation-related laboratory indicators. The improvement of APTT and ETP reached the plateau stage after four administrations in multiple injections of RVV-X at the dose of 0.16 U/kg. Neither serious adverse event (SAE) nor dose-limiting toxicity (DLT) events were reported. Neither thromboembolic events nor disseminated intravascular coagulation (DIC) were observed. Anti-drug antibody of the administration of RVV-X was not detected. These results indicated that RVV-X has potential to be used as an on-demand treatment for bleeding episodes in patients with hemophilia no mater HA or HB.

### Methods

### Study Design and Participants

This is a phase I, first-in-human, multi-center, open-label, dose-escalation study. The study was conducted at the Institute of Hematology & Blood Diseases Hospital, Chinese Academy of Medical Sciences (Tianjin, China) and Henan Cancer Hospital (Zhengzhou, China) in accordance with the Declaration of Helsinki and International Conference on Harmonisation Guidelines for Good Clinical Practice. The study was approved by the ethics committees at each participating center (XY2019039-EC-2 and XY2021034-EC-2) and registered at www.clinicaltrials.gov (NCT-04747964 and NCT-05027230). All subjects gave written informed consents before enrollment.

Chinese male patients (18 to 65 years old) who had moderate or severe hemophilia A or B (factor activity level <5% IU/dL), with factor VII or IX inhibitors respectively were eligible to be enrolled in the study. Subjects with previous or current history of clinically significant allergy to any component of the drug or protein blood products, coagulation disorders other than hemophilia A or B, thromboembolic diseases were excluded.

In single-dose part, eligible patients received a single intravenous (IV) injection of RVV-X in cohort 1 (0.01 U/kg, n=2), cohort 2 (0.04 U/kg, n=2), cohort 3 (0.08 U/kg, n=3), cohort 4 (0.16 U/kg, n=3), cohort 5 (0.32 U/kg, n=3) and cohort 6 (0.48 U/kg, n=3).

In multiple-dose part of 0.16 U/kg, eligible patients received at most six IV injections of RVV-X every four hours (q4h) in 0.16 U/kg (n=6). The dose of 0.16 U/kg was selected as the initial dose of multiple dose-escalation based on the study results of phase A which indicated that 0.16 U/kg of RVV-X can shorten APTT significantly. In further multiple-dose part of 0.1 U/kg, eligible patients also received at most six IV injections of RVV-X every four hours (q4h) in 0.1 U/kg (n=6).

The dose escalation was initiated from the lowest dose of RVV-X, and followed a stepwise dose-escalation procedure. At each dose in single-dose part, RVV-X was firstly only administered to 1 subject each. After confirming safety for 48 hours, RVV-X was administered to the remaining subjects. Vital signs, laboratory tests (see indicators in “Laboratory Tests” in Example 2), electrocardiograms (ECG), ultrasound (abdominal and deep veins of both lower limbs), adverse events were monitored for up to 168 hours after administration. After tolerability and safety were confirmed, the decision to proceed to the next dose step was made by the Data and Safety Monitoring Board. Multiple-dose part was conducted after confirming the tolerability and safety of single-dose part

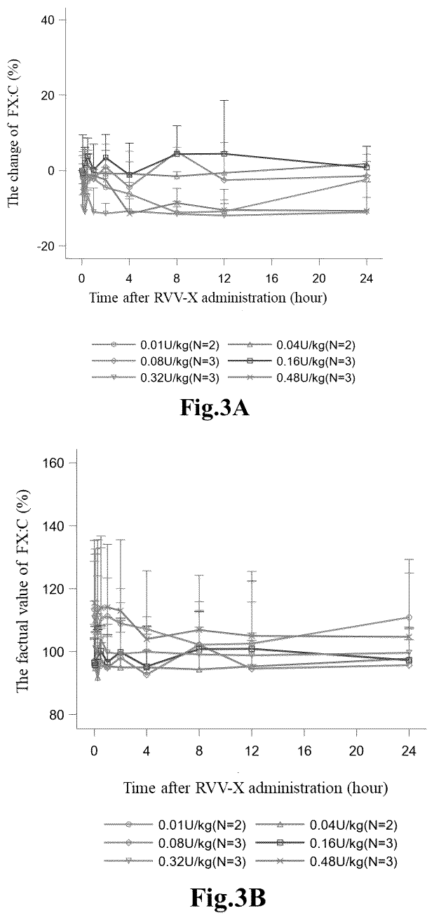

Minimal anticipated biological effect level (MABEL) was determined as 0.001 U/ml based on study results of the effect of RVV-X on APTT in FVIII deficient human plasma, FIX deficient human plasma, and normal human plasma, which indicated that 0.001 U/ml~10 U/ml of RVV-X could shorten APTT of FVIII deficient human plasma and FIX deficient human plasma in a dose-dependent manner (FIG. 4). The Maximum Recommended Starting Dose (MRSD) was estimated as 0.067 U/kg based on the average healthy human weight of 60 kg and circulating blood volume of 4000 ml.

### Study Drug and Administration

The formulation for administration comprises RVV-X (comprising SEQ ID NO: 1, SEQ ID NO: 2 and SEQ ID NO: 3) isolated and purified from the venom of Daboia russellii siamensis, and excipients. Excipients include sucrose, histidine, polysorbate 20, and mannitol.

The product is white or white-like sparse block or powder, sterile, for injection purpose. Each vial contains 5U of lyophilized RVV-X. Before injection, each vial was reconstituted with 2 ml of 0.9% sodium chloride injection solution. Reconstituted formulation was slowly injected intravenously to tested subjects, and the injection volume was calculated for each subject according to the dose of each group.

### Outcome Measures

Observation was performed for: vital signs, 12-lead electrocardiography, chest radiography, ultrasound (abdominal, deep vein of both lower limbs), factor VII concentration (FVIII:C), factor IX concentration (FLX:C), FVII or FLX inhibitor, blood routine, blood biochemistry, urine routine, prothrombin time (PT), APTT, fibrinogen (FIB), thrombin time (TT), factor X concentration (FX:C), thrombin generation (TG), D-dimer, fibrin degradation product (FDP), PK test of RVV-X, and anti-RVV-X antibodies.

### Pharmacokinetics (PK)

Plasma RVV-X concentrations were determined by a validated electrochemiluminescence assay (Meso Scale Discovery, America). The lower limit of quantification was 5pg/mL.

### Pharmacodynamics (PD)

APTT, factor X activity assay (FX:C), thrombin generation (TG) including the peak height, endogenous thrombin-generating potential (ETP), were evaluated. APTT was assessed using the APTT-ACTIN FSL kit (Siemens, Germany), the CS-5100 (Sysmex, Japan) automated blood coagulation analyzer or the STA-CK Prest (Stago, France) and the STA-R Max (Stago) automated blood coagulation analyzer. FX;C was assessed by the validated coagulation assay using the automated blood coagulation analyzer (Sysmex). TG was measured by the calibrated automated thrombogram assay using the TG (CAT) analyzer (Stago), with 1 pM tissue factor (TF) and 4 μM phospholipid (Stago) as triggers.

### Safety

Safety outcomes were assessed by vital signs, physical examination, laboratory test results, electrocardiogram (ECG), ultrasound (abdominal and deep veins of both lower limbs), and adverse events profile. The severity of adverse events was determined with reference to the NCI Common Terminology Criteria for Adverse Events (CTCAE v5.0).

### Immunogenicity

Anti-RVV-X binding antibody in plasma was screened and confirmed by a validated electrochemiluminescence assay (Meso Scale Discovery). For positive samples, the antibody titer and neutralizing antibody were further assessed.

### Statistical Analysis

Summary statistics were calculated for demographic characteristics, PK, PD, and safety outcomes. In single-dose part, at least two patients were planned to enrolled in each cohort to evaluate the safety, PK and PD. PK parameters including maximum plasma concentration (Cₘₐₓ), area under the plasma concentration-time curve from time 0 to last quantifiable concentration (AUC₀₋ₜ), area under the plasma concentration-time curve extrapolated to infinity (AUC_{0-∞}), time to reach maximum plasma concentration (Tₘₐₓ), elimination half-life (t_{1/2}), apparent volume of distribution (V_{z}), plasma clearance(CL_{z}), elimination rate constant (λ_{z}), percentage of AUC due to extrapolation from the time of the last quantifiable concentration to infinity (AUC_{% Extrap}) were calculated from each subject's RVV-X plasma concentration-time profile by a noncompartmental analysis using Phoenix WinNonlin software version 8.1. The relationship between AUC and dose, Cₘₐₓ and dose were analyzed by a power function model. In multiple-dose part, AUC₀₋ₜ, AUC_{0-∞}, t_{1/2} were calculated.

### Results

### Study Population

Single-dose part: A total of 16 Chinese hemophilia patients were enrolled in this trial. Among them, 15 patients were hemophilia A (HA) with inhibitors and one patient was hemophilia B (HB) with inhibitors (Table 1).

Multiple-dose part: For 0.16 U/kg, a total of 7 Chinese hemophilia patients were enrolled in this trial, of which 5 subjects received 5 doses, 1 subject received 6 doses and 1 subject received 2 doses. Among them, 5 patients were hemophilia A (HA) with inhibitors and 2 patient was hemophilia B (HB) with inhibitors. For 0.1 U/kg, a total of 6 Chinese hemophilia patients were enrolled in this trial, of which all 6 subjects received 6 doses. Among them, 5 patients were hemophilia A (HA) with inhibitors and 1 patient was hemophilia B (HB) with inhibitors. (Table 1).

All patients were Han nationality and completed the administration of RVV-X in accordance with the requirements of the protocol.

TABLE 1
Baseline Demographic and Clinical Characteristics of the Study Population

| | Single-dose part | | | | | | Multiple-dose part | |
|---|---|---|---|---|---|---|---|---|
| | 0.01 U/kg | 0.04 U/kg | 0.08 U/kg | 0.16 U/kg | 0.32 U/kg | 0.48 U/kg | 0.16 U/kg | 0.1 U/kg |
| | (N = 2) | (N = 2) | (N = 3) | (N = 3) | (N = 3) | (N = 3) | (N = 7) | (N = 6) |
| Age (year) | | | | | | | | |
| Median | 44.5 | 35.5 | 26.0 | 21.0 | 31.0 | 31.0 | 34 | 42 |
| Min~Max | 42~47 | 25~46 | 20~27 | 20~29 | 30~47 | 27 -- 31 | 22~48 | 25~49 |
| Weight (kg) | | | | | | | | |
| Median | 70.90 | 63.65 | 59.00 | 49.00 | 66.00 | 66.00 | 76.00 | 64.75 |
| Min ~~ Max | 68~73.8 | 55~72.3 | 56.1~61 | 40.4~89.4 | 51.7~97.7 | 39.1~85 | 59~100 | 59~81 |
| BMI (kg/m²) | | | | | | | | |
| Median | 25.45 | 22.14 | 19.19 | 18.22 | 25.46 | 23.11 | 25.991 | 22.81 |
| Min~Max | 24.66~ | 19.26~ | 19.04~ | 13.98~ | 18.99~ | 14.72~ | 21.41~ | 19.7~ |
| | 26.23 | 25.02 | 21.41 | 29.53 | 30.84 | 28.08 | 31.38 | 27.1 |
| Types of | | | | | | | | |
| hemophilia | | | | | | | | |
| A | 2 | 2 | 3 | 3 | 3 | 2 | 5 | 5 |
| B | 0 | 0 | 0 | 0 | 0 | 1 | 2 | 1 |
| Severity of | | | | | | | | |
| hemophilia | | | | | | | | |
| Severe n (%) | 2(100.0) | 1(50.0) | 2(66.7) | 1(33.3) | 1(33.3) | 2(66.7) | 7(100.0) | 6(100.0) |
| Moderate n (%) | 0(0) | 1(50.0) | 1(33.3) | 2(66.7) | 2(66.7) | 1(33.3) | 0(0) | 0(0) |

Classification of hemophilia severity. Severe: factor activity level (IU/dL) < 1%, Moderate: factor activity level (IU/dL) 1-5, Mild: factor activity level (IU/dL) > 5%.

### PK

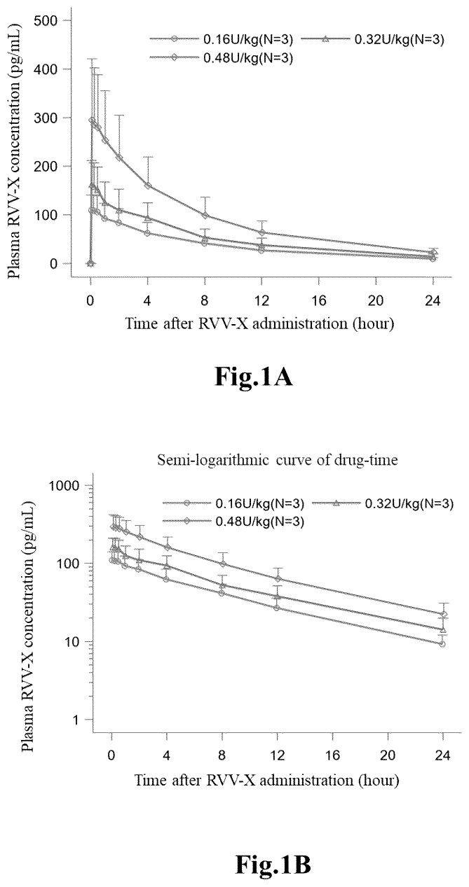

Single-dose part: The plasma concentration of RVV-X increased in a dose-dependent manner, and Cₘₐₓ increased in a linear PK characteristic with dose escalation at doses of 0.16 U/kg, 0.32 U/kg and 0.48 U/kg (FIGS. 1A-1C). Cₘₐₓ occurred at 5 minutes, and gradually returned to the pre-dose level after 24 hours post-dose. The results of the PK parameter analyses (Table 2-1) showed that the Cₘₐₓ were 110.04±32.03 pg/mL, 163.51±50.79 pg/mL, and 296.48±122.65 pg/mL respectively, for single-doses of 0.16 U/kg, 0.32 U/kg and 0.48 U/kg of RVV-X. The AUC_{0-∞} were 988.38±325.97 h*pg/mL, 1419.10±497.46 h*pg/mL, and 2478.88±944.98 h*pg/mL respectively, and the AUC_{0-∞} also increased with dose escalation. The T_{1/2} were 7.33±0.55 h, 8.33±0.41 h, and 7.57±0.68 h respectively.

TABLE 2-1
PK parameters of RVV-X after single injection

| | 0.16 U/kg | 0.32 U/kg | 0.48 U/kg |
|---|---|---|---|
| parameter | (N = 3) | (N = 3) | (N = 3) |
| Cₘₐₓ(pg/mL) | 110.04 ± 32.03 | 163.51 ± 50.79 | 296.48 ± 122.65 |
| AUC_{0-∞}(h*pg/mL) | 988.38 ± 325.97 | 1419.10 ± 497.46 | 2478.88 ± 944.98 |
| Tₘₐₓ(h) | 0.19 ± 0.10 | 0.19 ± 0.10 | 0.22 ± 0.24 |
| t_{1/2z}(h) | 7.33 ± 0.55 | 8.33 ± 0.41 | 7.57 ± 0.68 |
| V_{z}(×10⁻³ U/(pg/mL)/kg) | 1.84 ± 0.62 | 2.90 ± 0.85 | 2.41 ± 1.17 |
| CL_{z}(×10⁻⁴ U/(h*pg/mL)/kg) | 1.73 ± 0.52 | 2.44 ± 0.80 | 2.21 ± 1.08 |
| λ_{z}(1/h) | 0.09 ± 0.01 | 0.08 ± 0.00 | 0.09 ± 0.01 |
| AUC_{_{—}}_{%Extrap}(%) | 9.90 ± 1.22 | 12.00 ± 1.75 | 9.88 ± 1.84 |

Data are presented as mean ± standard deviation.

Multiple-dose part of 0.16 U/kg: The plasma concentration of RVV-X increased gradually with 5 consecutive doses, but the increasing trend was gradually gentle (FIG. 1D). The results of the PK parameter analyses in multiple injections of 0.16 U/kg showed that the AUC_{0-∞} was 3342.02 h*pg/mL, and the T_{1/2} was 8.40 hrs.

Multiple-dose part of 0.1 U/kg: The plasma concentration of RVV-X also increased gradually with 6 consecutive doses (Table 2-2). The results of the PK parameter analyses in multiple injections of 0.1 U/kg showed that the mean AUC_{0-∞} was 1580.93 h*pg/mL, and the T_{1/2} was 8.68 hrs.

TABLE 2-2
Concentration of RVV-X after multiple-injection (0.1 U/kg)
Mean Concentration of RVV-X (pg/mL)

| | | 10 min | 4 h | 4 h 10 min | 8 h | 8 h 10 min | 12 h | 12 h 10 min |
|---|---|---|---|---|---|---|---|---|
| | | after | after | after | after | after | after | after |
| | Base- | 1st | 1st | 1st | 1st | 1st | 1st | 1st |
| N = 6 | line | dose | dose | dose | dose | dose | dose | dose |
| Mean | 0.0 | 52.92 | 28.59 | 81.41 | 44.88 | 87.10 | 62.02 | 103.56 |
| (SD) | | (5.91) | (3.72) | (7.96) | (7.60) | (20.09) | (9.35) | (16.01) |

| | 16 h | 16 h 10 min | 20h | 20 h 10 min | 8h | 28 h | 36 h |
|---|---|---|---|---|---|---|---|
| | after | after | after | after | after | after | after |
| | 1st | 1st | 1st | 1st | 1st | 1st | 1st |
| N = 6 | dose | dose | dose | dose | dose | dose | dose |
| Mean | 66.50 | 119.88 | 69.41 | 119.94 | 50.57 | 9.73 | 25.29 |
| (SD) | (14.74) | (17.27) | (13.94) | (15.47) | (13.00) | (5.59) | (44.83) |

The PK data were consistent and comparable between single- and multiple-dose regimens.

### PD

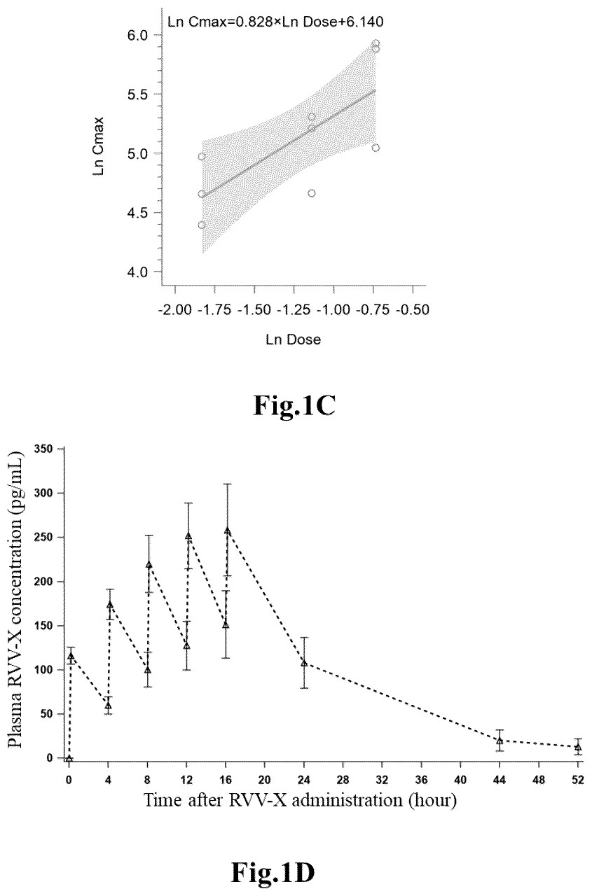

Single-dose part: After RVV-X administration, both the change of TG's peak height and ETP increased in a dose-dependent manner, reached peak at 5 min, remained at a high level until 4 hours, and gradually returned to the pre-dose level after 24 hours (FIGS. 2A and 2B). APTT was shortened in a dose-dependent manner, and the maximum shortening of APTT was observed at 5 minutes, lasted until 12 hours, and gradually returned to the pre-dose level after 24 hours (Table 3-1).

The improvement of APTT and ETP was more and more obvious with dose escalating of study drug in single-dose part, and RVV-X significantly improved the coagulation-related laboratory indicators (see indicators in “Laboratory Tests” in Example 2) starting from the dose of 0.16 U/kg.

TABLE 3-1
APTT in single-dose part

| | The value of APTT (s) | | | | | | | | | | | | | | | |
|---|---|---|---|---|---|---|---|---|---|---|---|---|---|---|---|---|
| Number | 0.01 U/kg | | 0.04 U/kg | | 0.08 U/kg | | | 0.16 U/kg | | | 0.32 U/kg | | | 0.4 8U/kg | | |
| of | (N = 2) | | (N = 2) | | (N = 3) | | | (N = 3) | | | (N = 3) | | | (N = 3) | | |
| patients | 1001 | 1002 | 2001 | 2002 | 3001 | 3002 | 3003 | 4001 | 4002 | 4003 | 5001 | 5002 | 5003 | 6001 | 6002 | 6003 |
| Baseline | 81.7 | 89.1 | 95.6 | 87.90 | 85.1 | 83.00 | 103.9 | 95.7 | 111.50 | 121.90 | 86.5 | 105.60 | 112.00 | 90.2 | 86.3 | 146.6 |
| 5 min | 88 | 89.1 | 91.5 | 77.10 | 83.3 | 67.60 | 99.1 | 88.4 | 71.10 | 90.90 | 77.4 | 75.70 | 80.20 | 73.7 | 67.8 | 83.90 |
| 15 min | 86 | 87.7 | 90.4 | 75.00 | 81.8 | 67.90 | 100.1 | 87.8 | 65.90 | 87.60 | 75.7 | 74.30 | 80.30 | 74.1 | 66.7 | 85.30 |
| 30 min | 86.3 | 89 | 92.2 | 77.70 | 82 | 67.50 | 97.7 | 85.8 | 66.80 | 90.90 | 75.4 | 74.50 | 81.40 | 72.1 | 61.8 | 84.60 |
| 1 h | 83.8 | 89.4 | 92.2 | 77.80 | 82 | 70.40 | 97.4 | 88.7 | 71.90 | 90.40 | 71.4 | 75.40 | 79.80 | 70.6 | 64.3 | 83.80 |
| 2 h | 85.2 | 84.4 | 89.8 | 77.90 | 80.2 | 65.90 | 96.7 | 85.6 | 68.40 | 91.40 | 69.6 | 74.60 | 69.60 | 69.6 | 60 | 84.00 |
| 4 h | 85.4 | 85.1 | 89.5 | 79.60 | 85 | 72.00 | 99.9 | 84 | 71.90 | 93.50 | 68.4 | 78.80 | 89.70 | 69.5 | 56.9 | 83.90 |
| 8 h | 87 | 86.8 | 86.3 | 81.90 | 77.1 | 73.50 | 90.4 | 80 | 84.50 | 83.70 | 69.1 | 86.00 | 92.80 | 67.6 | 55.2 | 83.30 |
| 12 h | 86.1 | 84.1 | 87.7 | 83.50 | 77.8 | 79.30 | 95.2 | 80.7 | 74.30 | 101.40 | 71.6 | 89.60 | 101.10 | 70.1 | 54.7 | 83.00 |
| 24 h | 83.5 | 87.4 | 92.2 | 85.90 | 81.8 | 86.70 | 104.4 | 88.9 | 94.30 | 111.70 | 82.8 | 97.30 | 110.20 | 83.9 | 70.9 | 166.00 |

Multiple-dose part of 0.16 U/kg: After the first three administrations of RVV-X, peak height of TG increased continuously, and it did not increase significantly after the fourth administration. After administration of RVV-X, APTT shortened rapidly (within 10m n of first administration, shortened ~22 a seconds on average compared to baseline level) and decreased to the plateau stage at 4 hours after the third administration. This indicates that 4 administrations of RVV-X has reached the maximum efficacy. The shortening of APTT continued until 8 hours after the last administration and gradually recovered to the pre-dose level at 28-36 hours after the last administration (Table 3-2).

Multiple-dose of 0.1 U/kg: After six administrations of R-X, peak height of TG increased continuously. After administration of RVV-X, APTT shortened rapidly (within 10 min of first administration, shortened ~7 seconds on average compared to baseline level and after 3 administrations, shortened ~23 seconds on average, compared to baseline level). The shortening of APTT continued until 8 hours after the last administration and gradually recovered to the pre-dose level at 36 hours after the last administration (Table 3-3).

Multiple-dose of 0.16 U/kg or 0.1 U/kg also significantly improved the coagulation-related laboratory indicators.

TABLE 3-2
APTT in multiple-dose part (0.16 U/kg)

| | The value of APTT (s) | | | | | | | | | | |
|---|---|---|---|---|---|---|---|---|---|---|---|
| | | 10 min | 10 min | 10 min | 10 min | 10 min | 10 min | 8 h | 28 h | 36 h | 168 h |
| Number | | after | after | after | after | after | after | after | after | after | after |
| of | | 1^{st} | 2^{nd} | 3^{rd} | 4^{th} | 5^{th} | 6^{th} | last | last | last | 1^{st} |
| patients | Baseline | dose | dose | dose | dose | dose | dose | dose | dose | dose | dose |
| 1001 | 83.1 | 76.8 | 65,3 | 58.6 | 53.8 | 54.4 | 51.7 | 56.2 | 72.7 | 78.9 | 81.8 |
| 1002 | 139.7 | 105 | 83.8 | 65.7 | 61.5 | 64 | — | 62.9 | 104.8 | 121.9 | 142.2 |
| 1003 | 98.5 | 86.5 | 72.6 | — | — | — | — | 68 | 86.1 | 124.8 | 130.1 |
| 1004 | 82.2 | 77.1 | 70.3 | 67.2 | 63.2 | 60.4 | — | 63.1 | 79.1 | 78.1 | 81.3 |
| 1005 | 92.5 | 49.5 | 51.9 | 50.5 | 45.8 | 43.8 | — | 47.2 | 69.3 | 80.9 | 86.6 |
| 1006 | 78.2 | 60.9 | 49 | 44 | 43.5 | 48.5 | — | 55 | 76.8 | 78.8 | 116.8 |
| 1007 | 83.7 | 74 | 67.3 | 59.5 | 60.5 | 59.1 | — | 63.2 | 81.4 | 83.9 | 82.3 |

Note:
— means there is no data in this time point.

TABLE 3-3
Mean APTT shorten in multiple-dose part (0.1 U/kg)

| | The value of APTT shortens (s) | | | | | | | | | | |
|---|---|---|---|---|---|---|---|---|---|---|---|
| | | | 4 h | 8 h | 12 h | 16 h | 20 h | | | | |
| | | 10 min | 10 m | 10 m | 10 min | 10 min | 10 min | 8 h | 28 h | 36 h | 168 h |
| | | after | in after | in after | after | after | after | after | after | after | after |
| | | 1^{st} | 1^{st} | 1^{st} | 1^{st} | 1^{st} | 1^{st} | last | last | last | 1^{st} |
| N = 6 | Baseline | dose | dose | dose | dose | dose | dose | dose | dose | dose | dose |
| Mean | 92.30 | −6.35 | −14.15 | −22.62 | −25.65 | −26.85 | −27.00 | −23.23 | −9.30 | −13.15 | 0.88 |

### Safety Outcomes

Single-dose part: RVV-X was well tolerated up to 0.48 U/kg during the study. A total of 36 adverse events were reported in 14 of the 16 patients (88%) (Table 4-1), among which 32 (89%) events were grade 1 in severity and 4 (11%) events were grade 2. There was no dose-limiting toxicity (DLT) event, serious adverse events (SAE), or adverse event (AE) that led to study withdrawal reported during the trial. Moreover, the incidence of AEs did not increase dose dependently. All drug-related AEs judged by the investigators were grade 1, and neither drug-related SAE nor Suspected Unexpected Serious Adverse Reaction (SUSAR) were reported.

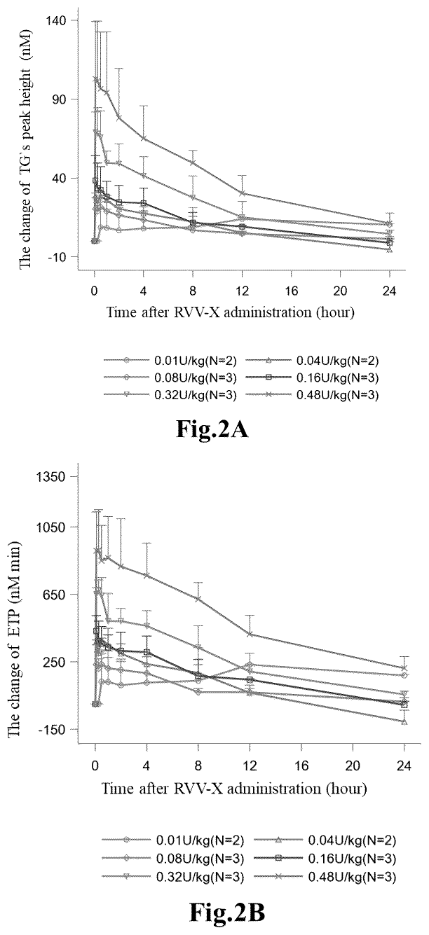

No evidence of clinically relevant coagulation abnormalities was noted, no lower extremity deep venous thrombosis (DVT) was found and no thromboembolic events were reported on the basis of reported AEs (Table 4-1). Within 24 hours after administration of RVV-X, FX showed a certain extent of decrease in each cohort, but the change from baseline was within 10% (FIG. 3A), and the actual measured value was in above 90% of normal value (FIG. 3B). This result indicated that activation of FX by RVV-X was very safe, and consumption of FX was minimal.

TABLE 4-1
All AEs of single-dose part

| | Number of patients (%) | | | | | | |
|---|---|---|---|---|---|---|---|
| Event | Cohorts of RVV-X | | | | | | |
| SOC | 0.01 U/kg | 0.04 U/kg | 0.08 U/kg | 0.16 U/kg | 0.32 U/kg | 0.48 U/kg | Total |
| PT | (n = 2) | (n = 2) | (n = 3) | (n = 3) | (n = 3) | (n = 3) | (n = 16) |
| Blood and lymphatic system disorders | | | | | | | |
| Anaemia | 0(0) | 0(0) | 1(33.3) | 0(0) | 1(33.3) | 0(0) | 2(12.5) |
| Cardiac disorders | 1(50.0) | 0(0) | 0(0) | 1(33.3) | 0(0) | 1(33.3) | 3(18.8) |
| Sinus bradycardia | 0(0) | 0(0) | 0(0) | 1(33.3) | 0(0) | 1(33.3) | 2(12.5) |
| Defect conduction intraventricular | 1(50.0) | 0(0) | 0(0) | 0(0) | 0(0) | 0(0) | 1(6.3) |
| Eye disorders | | | | | | | |
| Extraocular muscle paresis | 1(50.0) | 0(0) | 0(0) | 0(0) | 0(0) | 0(0) | 1(6.3) |
| Gastrointestinal disorders | | | | | | | |
| Diarrhoea | 0(0) | 1(50.0) | 0(0) | 0(0) | 0(0) | 0(0) | 1(6.3) |
| General disorders and administration | | | | | | | |
| site conditions | | | | | | | |
| Influenza like illness | 0(0) | 0(0) | 0(0) | 1(33.3) | 0(0) | 0(0) | 1(6.3) |
| Chest pain | 0(0) | 1(50.0) | 0(0) | 0(0) | 0(0) | 0(0) | 1(6.3) |
| Hepatobiliary disorders | | | | | | | |
| Hepatic function abnormal | 1(50.0) | 0(0) | 0(0) | 0(0) | 0(0) | 1(33.3) | 1(12.5) |
| Hepatomegaly | 0(0) | 0(0) | 0(0) | 0(0) | 0(0) | 1(33.3) | 1(6.3) |
| Infections and infestations | | | | | | | |
| Urinary tract infection | 0(0) | 0(0) | 1(33.3) | 0(0) | 0(0) | 0(0) | 1(6.3) |
| Investigations | | | | | | | |
| Fibrin D dimer increased | 0(0) | 0(0) | 1(33.3) | 0(0) | 1(33.3) | 0(0) | 2(12.5) |
| Fibrin degradation products increased | 0(0) | 0(0) | 1(33.3) | 0(0) | 1(33.3) | 0(0) | 2(12.5) |
| Blood bilirubin increased | 0(0) | 1(50.0) | 0(0) | 1(33.3) | 0(0) | 0(0) | 2(12.5) |
| Ultrasound scan abnormal | 1(50.0) | 0(0) | 0(0) | 0(0) | 0(0) | 0(0) | 1(6.3) |
| Antithrombin III decreased | 0(0) | 0(0) | 0(0) | 0(0) | 1(33.3) | 0(0) | 1(6.3) |
| Red blood cells urine positive | 1(50.0) | 0(0) | 0(0) | 0(0) | 0(0) | 0(0) | 1(6.3) |
| Urinary occult blood positive | 1(50.0) | 0(0) | 0(0) | 0(0) | 0(0) | 0(0) | 1(6.3) |
| Electrocardiogram QT prolonged | 1(50.0) | 0(0) | 0(0) | 0(0) | 0(0) | 0(0) | 1(6.3) |
| Blood fibrinogen decreased | 0(0) | 0(0) | 0(0) | 0(0) | 1(33.3) | 0(0) | 1(6.3) |
| Metabolism and nutrition disorders | | | | | | | |
| Hypokalacmia | 1(50.0) | 0(0) | 1(33.3) | 0(0) | 0(0) | 0(0) | 2(12.5) |
| Hypoalbuminaemia | 0(0) | 0(0) | 0(0) | 0(0) | 1(33.3) | 0(0) | 1(6.3) |
| Musculoskeletal and connective tissue | | | | | | | |
| disorders | | | | | | | |
| Musculoskeletal discomfort | 1(50.0) | 1(50.0) | 0(0) | 0(0) | 0(0) | 0(0) | 2(12.5) |
| Joint microhaemorrhage | 0(0) | 1(50.0) | 0(0) | 0(0) | 0(0) | 0(0) | 1(6.3) |
| Renal and urinary disorders | | | | | | | |
| Renal hydrocele | 0(0) | 0(0) | 1(33.3) | 0(0) | 0(0) | 0(0) | 1(6.3) |
| Pyelocaliectasis | 1(50.0) | 0(0) | 0(0) | 0(0) | 0(0) | 0(0) | 1(6.3) |
| Skin and subcutaneous tissue disorders | | | | | | | |
| Pruritus | 0(0) | 1(50.0) | 0(0) | 0(0) | 0(0) | 0(0) | 1(6.3) |

MedDRA V24.0 was used for coding.
Note:
SOC: System Organ Classification, PT: Preferred Term.

Multiple-dose part of 0.16 U/kg: Multiple injections of RVV-X was safe and tolerated. A total of 26 adverse events (AEs) were reported in 7 of the 7 patients (100%) (Table 4-2), among which 24 (92%) events were grade 1, 1 (4%) event was grade 2, and 1 (4%) event was grade 3. The AEs caused 6 subjects to stop the continuous administration, of which 1 case withdrew from the trial. The specific reasons were: the increase of D-dimer (protein fragment generated when a blood clot dissolves) and fibrin degradation product (FDP) in 4 cases, the decrease of fibrinogen in 1 case, and the coldness of right dorsal foot in 1 case. After 3-4 times of administration of RVV-X, D-dimer increased significantly, but the maximum value of most patients did not exceed 5 mg/L and decreased automatically after stopping administration. There was no thromboembolic event or disseminated intravascular coagulation (DIC) reported. Within 24 hours after administration of RVV-X, FX showed a certain extent of decrease, but the change from baseline was within 20%.

A total of 18 AEs related to the study drug RVV-X were reported in 6 subjects, including 17 cases of grade 1, and 1 case of grade 3. Most AEs recovered without treatment after RVV-X was stopped. The only grade 3 AE was decrease of fibrinogen (FIB) levels. The course of this AE: FIB decreased from 1.91 g/L to 0.92 g/L after 5 administrations of RVV-X; without treatment, FIB levels returned to 1.84 g/L after 3 hours. FIB decreased by no more than lg/L compared with the baseline in other subjects, and they were all within the range of normal values. Neither RVV-X-related SAE nor SUSAR was reported.

In the multiple-dose of 0.16 U/kg group, up to six doses of RVV-X did not cause thrombotic events, indicating that RVV-X has a good safety profile while correcting coagulation dysfunction in hemophilia patients. However, D-dimer increased significantly after 3-4 doses of RVV-X, so no more than 4 consecutive doses are more secure.

In the multiple-dose of 0.10 U/kg group: multiple injections of RVV-X were also safe and tolerated. A total of 11 adverse events (AEs) were reported in 6 patients (Table 4-2), among which 8 (73%) events were grade 1, 3 (27%) event was grade 2. A total of 4 AEs related to the study drug RVV-X were reported in 6 subjects, including 3 cases of grade 1, and 1 case of grade 2. Most AEs recovered without treatment after RVV-X was stopped. Neither RVV-X-related SAE nor SUSAR was reported. The results indicated that RVV-X at a multiple-dose of 0.1 U/kg has a better safety profile while correcting coagulation dysfunction in hemophilia patients, when compared with that at a multiple-dose of 0.16 U/kg.

TABLE 4-2
All AEs of multiple-dose part

| Event | | |
|---|---|---|
| SOC | Number of patients(%) | Number of patients(%) |
| PT | 0.16 U/kg (n = 7) | 0.1U/kg (n = 6) |
| Blood and lymphatic system disorders | | |
| Peripheral coldness | 1(14.3) | 0(0) |
| Cardiac disorders | | |
| Sinus bradycardia | 1(14.3) | 0(0) |
| Gastrointestinal disorders | | |
| Diarrhoea | 1(14.3) | 0(0) |
| Hepatobiliary disorders | | |
| Gallbladder polyp | 1(14.3) | 0(0) |
| Hepatic steatosis | 1(14.3) | 0(0) |
| Medical examination | | |
| Fibrinogen degradation products increased | 6(42.9) | 1(16.67) |
| Fibrin D dimer increased | 6(42.9) | 2(33.33) |
| Blood fibrinogen decreased | 3(14.3) | 1(16.67) |
| Abnormal ST segment of ECG | 1(14.3) | 0(0) |
| Metabolic and nutritional diseases | | |
| Hypokalemia | 1(14.3) | 1(16.67) |
| Hypertriglyceridemia | 3(42.9) | 1(16.67) |
| Skeletal muscle and connective tissue diseases | | |
| Pain in extremity | 1(14.3) | 1(16.67) |
| Kidney and Urologic Diseases | | |
| Nephrolith | 0(0) | 1(16.67) |
| Renal neoplasm | 0(0) | 1(16.67) |
| Infection Diseases | | |
| Upper respiratory tract infection | 0(0) | 1(16.67) |
| Diseases of Reproductive System | | |
| and the Breast | | |
| Benign prostatic hyperplasia | 0(0) | 1(16.67) |

FX is RVV-X's target, so the activation of FX by RVV-X could lead to consumption of certain amount of FX. Above results from both single-dose part and multiple-dose part (both in 0.16 U/kg group and 0.1 U/kg group) indicate that the activation of FX by RVV-X will not cause excessive consumption of FX.

### Immunogenicity

### Binding Antibody

Sing-dose part: One of 16 subjects had low titer (<30) for binding antibody, before and on the 8th day after RVV-X administration. However, no increasement of the inhibitor titer was observed for this subject. Hence, the positive result of binding antibody is not considered to be related to RVV-X, but probably related to previous usage of other procoagulant drugs. Both APTT and TG in this subject had improved after administration.

Multiple-dose part of 0.16 U/kg: One of 7 subjects had low titer (<30) for binding antibody, before and on the 7th day after RVV-X administration. However, no obvious increasement of the inhibitor titer was observed for this subject. Hence, the positive result of binding antibody is not considered to be related to RVV-X, but probably related to previous usage of other procoagulant drugs. Both APTT and TG in this subject had improved after administration. Multiple-dose part of 0.1 U/kg: No binding antibody was detected in all 6 subjects.

### Neutralizing Antibody

All subjects were negative for neutralizing antibody, both in single injection and multiple injection groups (both 0.16 U/kg group and 0.1 U/kg group).

These findings lead to a preliminary conclusion that RVV-X has low immunogenicity, and acquisition of hypersensitivity is unlikely to occur with RVV-X administration.

In summary, this first-in-human study of RVV-X demonstrated that intravenous injection of RVV-X at a single dose up to 0.48 U/kg and multiple injections at 0.16 U/kg or 0.1 U/kg were safety and well tolerated. Starting from the single dose of 0.16 U/kg, RVV-X significantly improved the coagulation-related laboratory indicators. The improvement of APTT and ETP reached the plateau stage after four administrations in multiple injections of RVV-X at 0.16 U/kg. In conclusion, these results indicated that RVV-X has potential to be used as an on-demand treatment for bleeding episodes in patients with hemophilia no mater HA or HB. The findings in this study will advance RVV-X into subsequent clinical trials to explore the efficacy and safety of treating bleeding events in hemophilia patients with inhibitor.

### REFERENCES

1. Astermark J. Overview of inhibitors. Semin Hematol. 2006:43: S3-S7.
2. Chevreux G, Faid V, Scohyers J M, Bihoreau N. N-/O-glycosylation analysis of human FVIIa produced in the milk of transgenic rabbits. Glycobiology. 2013:23:1531-1546.
3. Fenaille F, Groseil C, Ramon C, et al. Mass spectrometric characterization of N- and O-glycans of plasma-derived coagulation factor VII. Glycoconj J. 2008; 25:827-842.
4. Grandoni J, Perret G, Forier C. Kinetic analysis and binding studies of a new recombinant human factor VIIa for treatment of haemophilia. Haemophilia. 2017; 23:300-308.
5. Baxter HC. Anti-Inhibitor Coagulant Complex For Intravenous Use(FEIBA®) Label. 2013.11.
6. Parameswaran R, Shapiro A D, Gill J C, Kessler C M. Dose effect and efficacy of rFVIIa in the treatment of haemophilia patients with inhibitors: analysis from the Hemophilia and Thrombosis Research Society Registry. Haemophilia 2005; 11: 100-6.
7. Santagostino E, Mancuso M E, Rocino A, Mancuso G, Scaraggi F, Mannucci P M. A prospective randomized trial of high and standard dosages of recombinant factor VIa for treatment of hemarthroses in hemophiliacs with inhibitors. J Thromb Haemost 2006; 4: 367-71.
8. Gowda DC, Jackson CM, Hensley P, Davidson EA. Factor X-activating glycoprotein of Russell's viper venom. Polypeptide composition and characterization of the carbohydrate moieties. J Biol Chem. 1994. 269(14): 10644-50.
9. Takeda S, Igarashi T, Mori H. Crystal structure of RVV-X: an example of evolutionary gain of specificity by ADAM proteinases. FEBS Lett. 2007. 581(30): 5859-64.
10. Tans G, Rosing J. Snake venom activators of factor X: an overview. Haemostasis. 2001. 31(3-6): 225-33.
11. Drug levels, anti-drug antibodies, and clinical efficacy of the anti-TNFα biologics in rheumatic diseases.[J]. Clinical Rheumatology, 2013, 32(10):1429-1435.

### Example 2: Phase U Efficacy Study of FX Activator RVV-X in Hemophilia A or B Patients with Inhibitor

After investigator review, it was determined that in the phase I study 0.16 U/kg multiple-dose group was safe, in which APTT was significantly improved after study drug RVV-X was administered. Phase II trial (observational trial of efficacy in on-demand treatment of bleeding) with 0.16 U/kg multiple-dose regime was thus conducted.

The scale of the Phase II trial was further expanded: In addition to previously described 8 subjects enrolled with 19 bleeding events recorded, more subjects were enrolled in the trial (total N=35) and more bleeding events (total 171 events) were recorded.

Furthermore, 0.1 U/kg multiple-dose group is safer, with comparable APTT improvement. Therefore, 0.10 U/kg multiple-dose regime was also conducted in the trial (Total N=20).

### Study Description

The subjects enrolled in phase II trial are hemophilia patients with inhibitors. Subjects are given study drug treatment after bleeding events, and the efficacy is assessed.

Subjects who have completed phase I trial are allowed to continue to participate in the phase II trial after being evaluated by the investigators, and after informed consent from the subjects is obtained.

Follow-up can be performed if subjects are not bleeding at the time of enrollment. During the follow-up period, subjects who experience bleeding events (non-fatal or disabling bleeding episodes, excluding central nervous system bleeding and gastrointestinal bleeding) are immediately accepted by the study center to receive RVV-X treatment. Subjects with bleeding events at the time of enrollment should receive RVV-X hemostatic therapy immediately after enrollment.

Dosing regimen: continuous administration, with an interval of at least 4 hours between two administrations, until hemostasis, with a maximum of 6 administrations. Consolidation therapy with the study drug can be administered after clinical judgment of hemostasis (8 hours after the last administration, at most once, and no additional consolidation therapy is administered for those who have been administered 6 doses of RVV-X). If bleeding is still not effectively controlled within 4 hours after the end of 6 consecutive doses, and needs further treatment, the study drug should not be continued, and standard treatments recommended by the current clinical guidelines should be used instead for rescue treatment (such as recombinant human coagulation factor VIIa or PCC) and noted in record. If continuous administration of RVV-X is more than 2 times but less than 6 times, but the bleeding of the subjects continues to worsen during the administration period, rescue therapy can also be performed if the investigators determine that continued administration of RVV-X may fail to stop the bleeding and there may be a major medical risk.

After subjects are enrolled, they are followed up until the 12th week, and can be re-enrolled after being evaluated by the investigators and after informed consent from the subjects is received. For bleeding events occurred during the follow-up period, all patients are given on-demand RVV-X treatment according to the administration principle of first hemostasis, and dosage and dosing frequency are recorded to evaluate the hemostatic effect. If subjects are treated for bleeding events outside the study center, the bleeding events and medical treatment should be recorded in detail in the form of a diary card.

### Participants

Participants of the phase II study are hemophilia A or hemophilia B patients with factor VIM or IX inhibitors respectively.

Inclusion criteria: 1) Age 18-65 years old, male; 2) Hemophilia A or B patients; 3) Hemophilia patients with high inhibitor titer or high memory response: inhibitor titer is >5 BU/ml in at least one previous test, and inhibitor test is positive at the time of enrollment; 4) Ability to establish appropriate venous access; 5) The subjects have no reproductive plans during the trial period and within 3 months after the follow-up, and agree to take appropriate contraceptive measures to prevent their partners from becoming pregnant; and 6) Voluntarily participate and sign the informed consent.

Exclusion criteria: 1) Other coagulation disorders other than hemophilia A and B; 2) Hemophilia patients who plan to receive immune tolerance induction therapy or coagulation factor prophylaxis during the trial, or stop immune tolerance induction therapy or coagulation factor prophylaxis for less than 4 weeks; 3) Received anticoagulant or antifibrinolytic therapy within 7 days before study drug administration, or plan to use these drugs during the trial; 4) History of arteriovenous thrombosis, disseminated intravascular coagulation, myocardial infarction, cerebral infarction, thrombotic microangiopathy, autoimmune disease, malignant tumor, liver cirrhosis and other diseases, and determined as not suitable for the enrollment by investigators; 5) Thrombocytopenia (PLT<100×10⁹/L), moderate to severe anemia (hemoglobin<90 g/L); 6) Patients with liver disease who meet one of the following indicators: a) Total bilirubin ≥2 times the upper limit of the normal value, and b) Alanine aminotransferase (ALT) and aspartate aminotransferase (AST) ≥2 times the upper limit of the normal value; patients with kidney disease (serum creatinine ≥1.5 times the upper limit of normal); 7) Severe bleeding before the screening (fatal or disabling bleeding episodes, central nervous system bleeding, or gastrointestinal bleeding); 8) Received major surgical operation and received blood or blood component infusion within 4 weeks before screening; 9) Anti-human immunodeficiency virus (HIV) antibody tested positive, or with known HIV infection, and with CD4 count<200 cells/μL; 10) Those who are severely allergic to any component of the composition or other protein blood products; 11) Those who have participated in clinical trials of other new drugs within one month; 12) Used procoagulant drugs (such as prothrombin complex, recombinant human coagulation factor VIIa) within 7 days before study drug administration; or 13) The investigators think that they are not suitable to participate in this trial.

### Reference Criteria for Discontinuation of Dosing

During the process of multiple administration, if the test result of the subject meets any one of the following standards, study drug administration can be discontinued after the judgment by the investigators: i) fibrinogen (FIB) decreases by >50% of lower limit of normal value, or decreases by >1 g/L compared with baseline; ii) APTT returns to below the upper limit of normal value; iii) D-dimer is significantly higher than the baseline level and greater than 5 mg/L; and iv) thromboembolic event occurs.

Attention should be paid to the report of laboratory test results during the administration process, and safety judgments should be made timely after obtaining the test results.

### Study Drug and Administration

The formulation for administration comprises RVV-X (comprising SEQ ID NO: 1, SEQ ID NO: 2 and SEQ ID NO. 3) isolated and purified from the venom of Daboia russellii siamensis and excipients. Excipients include sucrose, histidine, polysorbate 20, and mannitol. Each vial contains 5U of lyophilized RVV-X. Before injection, each vial is reconstituted with 2 ml of 0.9% sodium chloride injection. Reconstituted formulation is slowly injected intravenously to tested subjects, and the injection volume is calculated for each subject according to the dose regime.

### Study Design

Screening period: Begins after the subject signs the informed consent form, and baseline data is collected.

Follow-up and treatment period: Subjects are admitted to the study center for RVV-X treatment immediately after a bleeding event (non-fatal or disabling bleeding episode).

The follow-up period is 12 weeks. After a subject completes the follow-up content of the 12th week after enrollment, it is defined as the subject's completion of the trial.

### Monitoring Indicators

### Routine Safety Monitoring Indicators

Physical examination: comprehensive physical examination.

Vital signs: blood pressure, pulse, respiration, temperature.

### Laboratory Tests

Blood routine: determination of white blood cell (WBC) count and classification (absolute neutrophil count (NEUT #), absolute lymphocyte count (LYMP #), absolute monocyte count (MONO #), absolute eosinophil count (EOS #), absolute basophil count (BASO #)), hemoglobin (HGB), red blood cell (RBC) count, hematocrit (HCT), and platelet (PLT) count.

Blood biochemistry: determination of alanine aminotransferase (ALT), aspartate aminotransferase (AST), total protein (TP), albumin (ALB), total bilirubin (TBIL), direct bilirubin (DBIL), alkaline phosphatase (ALP), blood glucose (GLU), triglyceride (TG), low density lipoprotein (LDL-C), lactate dehydrogenase (LDH), creatine kinase (CK), creatine kinase isoenzyme (CK-MB), creatinine (Cr), urea (Urea), uric acid (UA), potassium (K), sodium (Na), calcium (Ca), phosphorus (P), and chlorine (Cl).

Urine routine: morning urine to measure urine specific gravity (SG), pH, glucose (GLU), ketone bodies (KET), urine protein (PRO), white blood cells (LEU), occult blood (BLD), nitrite (NIT), Urobilinogen (URO), and Bilirubin (BIL).

Routine coagulation function: detection of prothrombin time (PT), activated partial thromboplastin time (APTT), thrombin time (TT), and fibrinogen (FIB).

Coagulation factor X (FX:C) and thrombin generation (TG) assay.

Fibrinolysis index detection: detection of D-dimer and fibrin degradation products (FDP).

FVIII and FIX activity, and inhibitor determination.

12-lead ECG.

Chest X-rav: check once during the screening period.

Infection screening: check once during the screening period.

B-ultrasound; including abdominal B-ultrasound and deep veins of lower extremity color ultrasound.

Immunogenicity screening: detection of anti-drug antibodies.

Blood drug concentration.

### Efficacy Endpoints

### Primary Efficacy Endpoints

Effective hemostasis rate (all bleeding events): judged according to the efficacy evaluation table (Table 5), and efficacy rating of good or above is considered as effective.

### Secondary Efficacy Endpoints

Clinical remission rate at 4 h/8 h/24 h after first dose (all bleeding events): significant pain relief and/or improvement in bleeding signs in patients after the first dose of treatment for each bleeding episode is determined as clinical remission.

### Statistical Analysis

Effective hemostasis rate and its 95% CI is calculated. Clinical remission rate and its 95% CI of 4 h, 8 h and 24 h after the first dosing for each bleeding episode is calculated.

### Results

Chinese male patients (18 to 65 years old) who had moderate or severe hemophilia A or B (factor activity level <5% IU/dL), with factor VIIM or IX inhibitors respectively were enrolled in the study. After the testing subjects experienced bleeding events, they were treated with the study drug RVV-X, then therapeutic efficacy was evaluated. RVV-X was administered at 0.16 U/kg once every 4 hours for 4-6 doses. For 0.1 U/kg group, RVV-X was administered at 0.1 U/kg once every 4 hours for 6 doses,

Among a total of 8 subjects experiencing a total of 19 bleeding events previously described, all bleeding events achieved hemostasis within 4 doses of RVV-X administration at 0.16 U/kg, and 16 bleeding events reached the standard of “effective hemostasis” specified in the clinical trial protocol (“effective hemostasis” is achieved when the efficacy grade is excellent or good, see Table 5). After the expanding of the trial, among a total of 35 subjects experiencing a total of 171 bleeding events, total 164 bleeding events reached the standard of “effective hemostasis” specified in the clinical trial protocol with the effective hemostasis rate of 95.91%. In addition, the effective hemostasis rate was as high as 86.21% after the first administration. Among the 164 effective hemostasis events, the mean times of administration required for effective hemostasis is 1.6±0.9.

Among a total of 20 subjects experiencing a total of 70 bleeding events, all bleeding events achieved hemostasis within 6 doses of RVV-X administration at 0.1 U/kg, and 66 bleeding events reached the standard of “effective hemostasis” specified in the clinical trial protocol with the effective hemostasis rate of 94.29% (“effective hemostasis” is achieved when the efficacy grade is excellent or good, see Table 5). In addition, the effective hemostasis rate was as high as 95.00% after the first administration, which is not statistically significant with that in 0.16 U/kg group (p=0.64). Among the 66 effective hemostasis events, the mean times of administration required for effective hemostasis is 2.2±1.2.

TABLE 5
Hemostasis efficacy grading scale

| Grade | Efficay |
|---|---|
| Excellent | Complete pain relief and/or improvement in bleeding signs within 8 hours of the first injection; |
| | no additional treatment is required within 24 hours after the end of the protocol-specified trial |
| | drug administration to relieve persistent signs at the same bleeding site |
| Good | Significant pain relief and/or improvement in bleeding signs within 8 hours of the first injection; |
| | at least 1 additional treatment within 24 hours after the end of the protocol-specified trial drug |
| | administration is needed to completely relieve the bleeding episode at the same bleeding site |
| Medium | Pain and/or bleeding signs begin to improve within 8 hours of the first injection; at least 1 |
| | additional treatment within 24 hours after the end of the protocol-specified trial drug |
| | administration is needed to partially relieve the bleeding episode at the same bleeding site |
| Ineffective | Pain and/or bleeding signs have no or minimal improvement within 8 hours of the first injection, |
| | or get worse |

The therapeutic effect of RVV-X (both at dose of 0.16 U/kg and 0.1 U/kg) was found to be significantly better than that of thrombin complex concentrates (PCC; effective hemostasis rate is 50% [see J. M. Lusher, “Controlled clinical trials with prothrombin complex concentrates,” Prog Clin Biol Res. 1984; 150:277-290]). The therapeutic effect of RVV-X was also found to be comparable to that of injectable recombinant human coagulation factor VIIa (effective hemostasis rate is about 90% [see C. Knight et al., “Systematic review of efficacy of rFVIIa and aPCC treatment for hemophilia patients with inhibitors,” Adv Ther. 2009; 26(1):68-88]).

### Example 3: Study of RVV-X in M-KOFVIII Hemophilia Mice

FDA approved activated recombinant factor VII (rFVIIa, e.g., NovoSeven® RT) acts by forming a complex with tissue factor, which activates FX to FXa via extrinsic pathway, which then converts prothrombin into thrombin to achieve hemostasis. NovoSeven® RT is commonly used for treating HA and HB patients with inhibitors. However, it is extremely pricy, thus having limited application. Further, non-clinical studies showed that NovoSeven® RT had short in vivo half-life: t_{1/2} is 0.5 hr in rats, and t_{1/2} is about 3 hrs in dogs and monkeys. Pulmonary vascular coagulation was seen in mice acutely administered with NovoSeven® RT; and pulmonary thrombus and local irritation at injection sites were seen in both rats and cynomolgus monkeys repeatedly administered with NovoSeven® RT. See, e.g., NovoSeven: EPAR—SCIENTIFIC DISCUSSION (https://www.ema.europa.eu/en/medicines/human/EPAR/novoseven), and NovoSeven® RT ill Prescribing Information (revised 7/2020; https://www.novomedlink.com/rare-bleeding-disorders/products/treatments/novosevenrt/about/why-novosevenrt.html).

This Example demonstrates that RVV-X has comparable or even superior therapeutic effects in vivo, compared to FDA approved NovoSeven® RT.

### Pre-Test of Procoagulant Effect of RVV-X for Injection on M-KOFVIII Hemophilia Mice

M-KOFVIII (FVII gene knockout) hemophilia mice were used in the experiments. RVV-X (comprising SEQ ID NO: 1, SEQ ID NO: 3 and SEQ ID NO: 5) was administered by caudal vein injection twice on Day 1 and Day 5 with the dosage of 0.1 U/kg, 1 U/kg, 10 U/kg, and 100 U/kg. The amount and time of bleeding were evaluated 30 min after the first administration, and prothrombin time (PT) and activated partial thromboplastin time (APTT) were evaluated 30 min after the second administration. NovoSeven® was used as positive control. RVV-X diluent served as placebo (PBO).

As shown in Table 6, M-KOFVIII mice exhibited significantly higher APTT than normal mice. NovoSeven® treatment in M-KOFVIII mice reduced both bleeding amount and bleeding time (data not shown), and APTT was rescued to normal animal level (Table 6). Single intravenous injection of 0.1 U/kg RVV-X already showed the tendency of shortening APTT in M-KOFVIII hemophilia mice compared to non-treated animals (41.7±14.9s vs. 55.7±10.8s). A dosage-dependent shortening of APTT was seen for single intravenous injection with the dosage of 0.1 U/kg-100 U/kg of RVV-X (Table 6). RVV-X also reduced bleeding time and bleeding amount of M-KOFVIII mice to varying degrees (data not shown). 0.1 U/kg RVV-X was considered as pharmacologically active dose (PAD) for M-KOFVIII hemophilia mice.

TABLE 6
Effects of RVV-X on APTT and PT of M-KOFVIII hemophilia mice

| | | | | | Number of animals | | Number of animals | |
|---|---|---|---|---|---|---|---|---|
| Group | Test/Control | Strain | Dosage | | tested for PT (n) | PT (s) | tested for APTT (n) | APTT^{Δ} (s) |
| 1 | RVV-X diluent | normal | — | | 4 | 9.2 ± 0.2 | 4 | 20.1 ± 1.6* |
| 2 | RVV-X diluent | hemophilia | — | | 4 | 9.3 ± 0.4 | 5 | 55.7 ± 10.8* |
| 3 | NovoSeven ® | hemophilia | 3 | mg/kg | 3 | 6.5 ± 0.1 | 4 | 20.4 ± 2.3^{▴} |
| 4 | RVV-X | hemophilia | 0.1 | U/kg | 2 | 9.2 ± 0.4 | 3 | 41.7 ± 14.9* |
| 5 | RVV-X | hemophilia | 1 | U/kg | 4 | 9.3 ± 0.3 | 4 | 25.3 ± 7.0^{▴} |
| 6 | RVV-X | hemophilia | 10 | U/kg | 2 | 8.9 ± 0.5 | 4 | 19.0 ± 11.0^{▴} |
| 7 | RVV-X | hemophilia | 100 | U/kg | 3 | 9.3 ± 1.1 | 3 | 27.2 ± 12.8^{▴} |

^{Δ}APTT was assessed 30min after single intravenous injection of RVV-X, presented as mean ± standard deviation;
*P < 0.05 compared with group 1;
^{▴}P < 0.05 compared with group 2;
APTT, activated partial thromboplastin time;
NovoSeven ®, an activated recombinant factor VII (rFVIla), has been approved by FDA for treating hemophilia A or B with inhibitors.

### Effect of RVV-X for Injection on APTT of M-KOFVIII Hemophilia Mice

C57BL/6 mice and M-KOFVII hemophilia mice were used in the experiment. Blood was collected from abdominal aorta 30 min after single caudal vein administration of test agent or control, and APTT was measured by coagulation method. NovoSeven® served as positive control. RVV-X diluent served as placebo (PBO).

As shown in Table 7, M-KOFVIII hemophilia mice exhibited significantly higher APTT than normal mice. NovoSeven® treatment in M-KOFVIII mice significantly reduced APTT, close to but still higher than normal animal level. Single intravenous injection of 0.25 U/kg-4 U/kg RVV-X significantly shortened APTT in M-KOFVIII mice in a dose-dependent manner. Further, single intravenous injection of 4 U/kg RVV-X shortened APTT even more than NovoSeven®, and reduced APTT to normal animal level. RVV-X at single dose of 1 U/kg could provide significant shortening of APTT equivalent to 3 mg/kg of NovoSeven® in M-KOFVIII hemophilia mice.

TABLE 7
Effects of RVV-X for injection on APTT of M-KOFVIII hemophilia mice

| Group | Test/Control | Dosage | | Strain | Animal Number (n) | APTT^{Δ} (s) |
|---|---|---|---|---|---|---|
| 1 | RVV-X diluent | — | | C57BL/6 mice | 12 | 17.70 ± 1.29 |
| 2 | RVV-X diluent | — | | Hemophilia mice | 12 | 53.37 ± 12.04* |
| 3 | NovoSeven ® | 3 | mg/kg | | 10 | 23.95 ± 11.70^{▴} |
| 4 | RVV-X | 4 | U/kg | | 12 | 18.89 ± 9.35^{▴} |
| 5 | RVV-X | 1 | U/kg | | 10 | 28.05 ± 4.62*^{▴} |
| 6 | RVV-X | 0.25 | U/kg | | 12 | 35.38 ± 7.11*^{▴□} |

^{Δ}APTT was assessed 30 min after single intravenous injection of RVV-X;
NA, not applicable;
*P ≤ 0.05 compared with group 1;
^{▴}P ≤ 0.05 compared with group 2;
^{□}P ≤ 0.05 compared with group 3.

Effects of RVV-X for Injection on Bleeding Volume, Bleeding Time, and 24 h Mortality after Tail Docking M-KOFVIII Hemophilia Mice

Bleeding amount and bleeding time are affected by many factors, such as the number and quality of platelets, the structure and function of capillaries, the interaction between platelets and capillaries, and coagulation factors. While 24 hrs mortality after tail docking is less affected by such factors, and is also closer to clinical evaluation indicators, thus is used as the main evaluation index of this experiment.

C57BL/6 mice and M-KOFVII hemophilia mice were used in the experiment. Test agent or placebo was administered by single caudal vein injection. 30 min after administration, tail docking was performed, and bleeding amount and bleeding time were recorded. Afterwards, animals were returned to original cages to continue feeding. The next day, mortality of test animals was recorded. NovoSeven® served as positive control. RVV-X diluent served as placebo.

As shown in Table 8, normal mice showed 0% mortality 24 hrs after tail docking, while M-KOFVIII hemophilia mice showed 66.7% mortality. NovoSeven® administration significantly reduced 24 hrs mortality to 8.3% in M-KOFVIII hemophilia mice. Single intravenous injection of 0.25 U/kg RVV-X already demonstrated the ability to reduce 24 hrs mortality in M-KOFVIII hemophilia mice. Single intravenous injection of 0.25 U/kg-4 U/kg RVV-X significantly inhibited bleeding tendency of M-KOFVII hemophilia mice (data not shown), and reduced 24 hrs mortality in a dose-dependent manner. Further, M-KOFVIII hemophilia mice receiving single intravenous injection of 4 U/kg RVV-X showed 0% mortality like normal mice, which was even more effective than NovoSeven®.

TABLE 8
Effects of RVV-X for injection on 24 hrs mortality
after tail docking of M-KOFVIII hemophilia mice

| Group | Test/Control | Dosage | | Strain | Number (n) | Mortality (%) |
|---|---|---|---|---|---|---|
| 1 | RVV-X diluent | — | | C57BL/6 mice | 12 | 0.0% |
| 2 | RVV-X diluent | — | | Hemophilia mice | 12 | 66.7% |
| 3 | NovoSeven ® | 3 | mg/kg | | 12 | 8.3% |
| 4 | RVV-X | 4 | U/kg | | 11 | 0.0% |
| 5 | RVV-X | 1 | U/kg | | 12 | 33.3% |
| 6 | RVV-X | 0.25 | U/kg | | 12 | 50.0% |

### Effects of RVV-X for Injection at Different Time Noints on APTT in M-KOFVIII Hemouhilia Mice

This experiment investigated the time-effect relationship of RVV-X for injection on coagulation function in M-KOFVII hemophilia mice. C57BL/6 mice (normal control) and M-KOFVM hemophilia mice were used in the experiment. Hemophilia mice were given single caudal vein injection of 1 U/kg RVV-X. APTT was examined at 5 min, 10 min, 30 min, 1 h, 2 h, 4h, 6h, and 8 h after administration, 8 mice/time point.

As shown in Table 9, APTT in M-KOFVII hemophilia mice significantly increased compared to normal mice. Single intravenous injection of RVV-X at dosage of 1 U/kg could significantly shorten APTT in M-KOFVIII hemophilia mice as early as 5 min after administration. APTT shortening effect lasted until 2 hrs post-administration, and returned to pre-administration level 4 hrs post-administration, demonstrating obvious time-effect relationship.

TABLE 9
Effects of RVV-X for injection at different time
points on APTT of M-KOFVIII hemophilia mice

| Time point | Strain | Number (n) | APTT (s) |
|---|---|---|---|
| Pre-administration | C57BL/6 mice | 8 | 21.2 ± 8.3 |
| Pre-administration | Hemophilia mice | 8 | 46.1 ± 7.3* |
| Post-administration 5 min | | 8 | 32.2 ± 16.7*^{#} |
| Post-administration 10 min | | 8 | 25.7 ± 2.3^{#} |
| Post-administration 30 min | | 8 | 30.1 ± 11.1*^{#} |
| Post-administration 1 h | | 8 | 31.5 ± 5.6*^{#} |
| Post-administration 2 h | | 8 | 31.8 ± 5.3*^{#} |
| Post-administration 4 h | | 8 | 40.6 ± 4.2* |
| Post-administration 6 h | | 8 | 38.3 ± 3.5* |
| Post-administration 8 h | | 8 | 46.0 ± 12.7* |

Note:
*indicates P < 0.05 compared with C57BL/6 pre-administration group; ^{#}indicates P < 0.05 compared with hemophilia mice pre-administration group.

Based on above in vivo efficacy test, single intravenous injection of RVV-X in M-KOFVIII hemophilia mice could significantly shorten APTT. A clear tendency in shortening APTT could already be seen at low dose of 0.1 U/kg. At 0.25 U/kg-4 U/kg dose range, significant shortening of APTT could be observed in a dose-dependent manner, bleeding tendency could be inhibited, and mortality after tail docking could be greatly reduced. Time-effect study revealed that single intravenous injection of RVV-X at 1 U/kg could significantly shorten APTT in M-KOFVIII hemophilia mice within 5 min to 2 hrs post-administration, and such effect recovered to pre-administration level 4 hrs post-administration.

Toxicology study of repeated administration of RVV-X by intravenous injection at a dose of 5 U/kg per day for 4 weeks in M-KOFVi hemophilia mice showed that there were no other systemic toxic reactions except local thrombosis at injection site. Therefore, the safe dose in mice was determined to be 5 U/kg.

### Example 4: Phase I Clinical Study of Multiple Doses of FX Activator for Injection in Surgery Patients

Safety and tolerability study was conducted in surgery patients with multiple administration of FX activator RVV-X for injection. Two or three types of surgery patients were enrolled as study population, and divided into several dose groups. A total of 2 doses were administered: once at 10-15 minutes before the surgery, and once during the surgery (1.5 hrs after the first dose), via intravenous administration. 3 days of observation was conducted after the surgery, then the study was completed.

A total of 18 subjects were enrolled in the trial, including 6 males and 12 females, among which 2 patients undergoing general surgery, 5 patients undergoing urological surgery, and 11 patients undergoing gynecological surgery.

A total of 9 (13 cases) adverse events occurred during the trial, which were all mild, including 7 cases of increased white blood cells, 1 case of decreased red blood cells, 1 case of decreased hemoglobin, 1 case of increased coagulation function international normalized ratio (INR), 1 case of lengthened coagulation function prothrombin time (PT), 1 case of rash, and 1 case of left upper pulmonary nodule shadow. Among them, PT prolongation and rash might be related to the study drug, and the others were not related to the study drug. Except for the left upper pulmonary nodule shadow, all other adverse events returned to normal at the end of the trial. No serious adverse events (SAE) occurred during the trial. No adverse events leading to the termination of the trial occurred.

Hence, FX activator for injection was well tolerated in all doses administered in surgery patients.

Efficacy study was also conducted in surgery patients with multiple administration of FX activator RVV-X for injection. Dozens of surgery patients were enrolled as study population, and administrated with RVV-X via intravenous administration. Observation was conducted after the surgery.

RVV-X exhibits therapeutic effect in surgery patients (data no shown).

### Example 5: Preparation of Coagulation Factor X Activator Injection Powder

The stock solution of coagulation factor X (FX) activator was from Russell's viper venom, which was prepared according to e.g Example 6 of CN109943554A (the content of which is incorporated herein by reference in its entirety). Other ingredients used were injection level unless otherwise specified.

Stabilizer, buffering agent, tonicity agent, surfactant, antioxidant, and divalent calcium salt (Ca²⁺) according to the prescription of Table 10A were weighed. Appropriate amount of water of injection level was added, and stirred until ingredients completely dissolved (when surfactant is liquid, other components can be dissolved first, then adding liquid surfactant and stirring well). FX activator stock solution was then added, and adjusted pH with HCl or NaOH. Finally, water of injection level was added to the set volume, and mixed well. Mixed solution was filtered into a sterile container via 0.22 μM filter membrane, to arrive at semi-finished solution.

TABLE 10A
FX activator Formulations 1-10

| | | Formulation # | | | | | | | | | |
|---|---|---|---|---|---|---|---|---|---|---|---|
| Ingredient | Function | 1 | 2 | 3 | 4 | 5 | 6 | 7 | 8 | 9 | 10 |
| FX activator | Active ingredient | 50 | 50 | 10 | 1 | 5 | 10 | 100 | 5 | 50 | 1 |
| | (U/ml) | | | | | | | | | | |
| Human serum | Stabilizer (mg/ml) | 1 | | | | | | | | | |
| albumin (HSA) | | | | | | | | | | | |
| Sucrose | | | 30 | | 15 | | 50 | 50 | 40 | | |
| Trehalose | | | | 60 | | 30 | | | | 30 | 40 |
| Histidine | Buffering agent | | 5 | | 10 | | 4 | | | | 4 |
| Arginine | (mg/ml) | | | 3 | | 4 | | 2 | 3 | 15 | |
| Glycine | | 20 | | | | | | | | | |
| Polysorbate 20 | Surfactant (mg/ml) | | 0.1 | | | | 0.3 | | 0.3 | | |
| Polysorbate 80 | | | | 0.05 | | 0.5 | | | | | 0.1 |
| Poloxamer 188 | | | | | 0.1 | | | 0.2 | | 0.5 | |
| Mannitol | Tonicity agent (mg/ml) | 50 | 60 | 10 | 55 | 40 | 40 | 30 | 30 | 40 | 30 |
| Methionine | Antioxidant (mg/ml) | | | 0.05 | | 1 | | | 0.3 | | |
| Calcium | Ca²⁺ (mg/ml) | | | | 1 | 10 | | | | | 3 |
| chloride | | | | | | | | | | | |
| pH | | 6.85 | 6.85 | 6.80 | 6.00 | 6.30 | 6.80 | 7.30 | 7.00 | 6.50 | 8.00 |

FX activator Formulations 1-10 were prepared according to Table 10A (Formulation 1 served as control), dispensed into medium borosilicate glass injection bottles at 0.5310.03 mi/bottle, plug half-pressed, then freeze-dried to obtain FX activator injection powder (hereinafter also referred as “FX activator Formulations 1-10 injection powder”).

The steps and parameters of freeze-dried process (also referred as lyophilization) was described in Table 10B.

TABLE 10B
Process parameters of Lyophilization

| | | | Time needed to | | |
|---|---|---|---|---|---|
| | | Set | achieve set | | |
| | | Temperature | temperature | Duration | Pressure |
| Step | Stage | (° C.) | (min) | (min) | (mbar) |
| Freezing | Stage 1 | −3 | 1 | 60 | — |
| | Stage 2 | −45 | 1 | 180 | — |
| Primary Drying | Stage 2 | −30 | 1 | 600 | 0.15 |
| Second Drying | Stage 1 | 30 | 120 | 120 | 0.20 |
| | Stage 2 | 30 | 1 | 120 | 0.20 |

“—” indicates no need to control pressure

### Example 6: Performance Test of Coagulation Factor X Activator Formulations

### I) Test of Appearance, Moisture, Reconstitution Time, DH, and Biological Activity

The appearance, moisture content, reconstitution time, pH value, and biological activity of FX activator Formulations 1-10 injection powder were examined. Biological activity was detected by coagulation meter. Results are shown in Table 11.

TABLE 11
Test Results of FX activator Formulations 1-10

| | | | Reconstitution | | Biological activity (relative |
|---|---|---|---|---|---|
| Formulation | Appearance* | Moisture (%) | time (s) | pH | to labeled amount, %) |
| 1 | Level 1 | 1.3 | 5 | 6.83 | 101.0 |
| 2 | Level 1 | 2.1 | 7 | 6.81 | 99.7 |
| 3 | Level 2 | 2.8 | 13 | 6.82 | 99.8 |
| 4 | Level 1 | 1.7 | 6 | 6.03 | 96.5 |
| 5 | Level 1 | 2.1 | 8 | 6.21 | 99.6 |
| 6 | Level 1 | 2.0 | 7 | 6.81 | 99.8 |
| 7 | Level 1 | 2.3 | 8 | 7.26 | 99.8 |
| 8 | Level 1 | 2.1 | 8 | 7.02 | 99.5 |
| 9 | Level 1 | 2.7 | 8 | 6.51 | 99.7 |
| 10 | Level 1 | 2.4 | 7 | 7.93 | 99.5 |

For better intuitive description, the appearance was divided into four levels. Level 1: exquisite, full, white loose body. Level 2: slight shrinkage, slightly rough surface, with particles, slightly off-wall. Level 3: moderate shrinkage, off-wall, blocks shrink slightly. Level 4: severe shrinkage, blocks shrink obviously, off-wall severely.

As shown in Table 11, the appearance, moisture content, reconstitution time, pH value, and biological activity of tested formulations all met corresponding product standards. Except for Formulation 3, the appearances of all other injection powder formulations were exquisite, full, white oose body, and could be quickly dissolved into colorless and clear liquid after reconstituting with 2 ml solvent. Due to the high content of trehalose and low content of mannitol, Formulation 3 injection powder had slight shrinking appearance, moisture content was close to 3.0%, reconstitution time was slightly longer than other formulations but still within 30 seconds, and was able to dissolve into colorless and clear liquid. There was no significant difference in pH and biological activity before and after lyophilization for all formulations.

### ID Stability Test Under High Temperature, High Humidity, and Light Conditions

Since the formulations may encounter conditions that deviate from the preset conditions during storage, we used influence factor test to investigate the impact on key indexes of formulations under a short period of exposure to high temperature (40±2° C.), high humidity (2-8° C., relative humidity (RH) 92.5%±5%), and light (2-8° C., 4500±500LX (lux as unit of measurement of light level intensity)). Test results are shown in Table 12 and Table 13.

TABLE 12
Partial indexes under the condition of influence factors

| | | | Formulation | | | | | | | | | |
|---|---|---|---|---|---|---|---|---|---|---|---|---|
| Condition | Time | Index | 1 | 2 | 3 | 4 | 5 | 6 | 7 | 8 | 9 | 10 |
| | Day | Appearance | Level | Level | Level | Level | Level | Level | Level | Level | Level | Level |
| | 0 | | 1 | 1 | 2 | 1 | 1 | 1 | 1 | 1 | 1 | 1 |
| | | Moisture | 1.3 | 2.1 | 2.8 | 1.7 | 2.1 | 2.0 | 2.3 | 2.1 | 2.7 | 2.4 |
| | | Reconstitution | 5 | 7 | 13 | 6 | 8 | 7 | 8 | 8 | 8 | 7 |
| | | time (s) | | | | | | | | | | |
| | | pH | 6.83 | 6.81 | 6.82 | 6.03 | 6.21 | 6.81 | 7.26 | 7.02 | 6.51 | 7.93 |
| High | Day | Appearance | Level | Level | Level | Level | Level | Level | Level | Level | Level | Level |
| temperature | 7 | | 1 | 1 | 3 | 1 | 2 | 1 | 2 | 1 | 2 | 2 |
| | | Moisture | 1.5 | 2.2 | 2.9 | 1.6 | 2.2 | 2.1 | 2.3 | 2.5 | 2.7 | 2.5 |
| | | Reconstitution | 5 | 7 | 16 | 8 | 10 | 7 | 8 | 7 | 9 | 9 |
| | | time (s) | | | | | | | | | | |
| | | pH | 6.85 | 6.83 | 6.80 | 6.03 | 6.28 | 6.81 | 7.3 | 7.01 | 6.5 | 7.98 |
| | Day | Appearance | Level | Level | Level | Level | Level | Level | Level | Level | Level | Level |
| | 14 | | 2 | 1 | 3 | 1 | 2 | 1 | 2 | 1 | 2 | 3 |
| | | Moisture | 1.5 | 2.3 | 2.9 | 1.8 | 2.3 | 2.1 | 2.3 | 2.4 | 2.8 | 2.6 |
| | | Reconstitution | 7 | 6 | 17 | 6 | 10 | 8 | 10 | 8 | 9 | 10 |
| | | time (s) | | | | | | | | | | |
| | | pH | 6.85 | 6.83 | 6.81 | 6.01 | 6.27 | 6.81 | 7.28 | 7.01 | 6.5 | 7.98 |
| High | Day | Appearance | Level | Level | Level | Level | Level | Level | Level | Level | Level | Level |
| humidity | 7 | | 1 | 1 | 2 | 1 | 1 | 1 | 1 | 1 | 1 | 1 |
| | | Moisture | 1.4 | 2.0 | 2.7 | 1.7 | 2.0 | 2.1 | 2.4 | 2.2 | 2.6 | 2.4 |
| | | Reconstitution | 6 | 6 | 14 | 7 | 9 | 6 | 00 | 8 | 7 | 8 |
| | | time (s) | | | | | | | | | | |
| | | pH | 6.86 | 6.82 | 6.79 | 6.01 | 6.26 | 6.78 | 7.29 | 7.02 | 6.49 | 7.96 |
| | Day | Appearance | Level | Level | Level | Level | Level | Level | Level | Level | Level | Level |
| | 14 | | 1 | 1 | 2 | 1 | 1 | 1 | 1 | 1 | 1 | 1 |
| | | Moisture | 1.4 | 2.1 | 2.6 | 1.9 | 2.0 | 2.2 | 2.2 | 2.4 | 2.5 | 2.5 |
| | | Reconstitution | 6 | 8 | 12 | | 7 | 8 | 9 | 9 | 9 | 6 |
| | | time (s) | | | | | | | | | | |
| | | pH | 6.79 | 6.84 | 6.81 | 5.99 | 6.26 | 6.79 | 7.25 | 7.02 | 6.51 | 7.93 |
| Light | Day | Appearance | Level | Level | Level | Level | Level | Level | Level | Level | Level | Level |
| | 7 | | 1 | 1 | 2 | 3 | 1 | 1 | 1 | 1 | 1 | 1 |
| | | Moisture | 1.2 | 2.0 | 2.8 | 1.6 | 2.1 | 2.1 | 2.5 | 2.2 | 2.6 | 2.7 |
| | | Reconstitution | 6 | OC | 12 | 7 | 8 | 7 | 7 | 9 | 7 | 8 |
| | | time (s) | | | | | | | | | | |
| | | pH | 6.85 | 6.83 | 6.77 | 5.99 | 6.31 | 6.82 | 7.29 | 7.01 | 6.49 | 7.98 |
| | Day | Appearance | Level | Level | Level | Level | Level | Level | Level | Level | Level | Level |
| | 14 | | 1 | 1 | 2 | 3 | 1 | 1 | 1 | 1 | 1 | 1 |
| | | Moisture | 1.3 | 2.0 | 2.7 | 1.6 | 2.2 | 2.1 | 2.2 | 2.2 | 2.8 | 2.5 |
| | | Reconstitution | 7 | 6 | 12 | 8 | 9 | 9 | 6 | 9 | 9 | 6 |
| | | time (s) | | | | | | | | | | |
| | | pH | 6.84 | 6.87 | 6.76 | 5.99 | 6.21 | 6.83 | 7.26 | 6.99 | 6.52 | 7.96 |

As shown in Table 12, under high temperature, all formulations met corresponding product standards as storage time increased. The appearance of Formulations 3 and 10 showed shrinking trend, and reconstitution time prolonged slightly. Under other testing conditions, there was no significant change in appearance, moisture, and pH for Formulations 2 and 4-9, compared with day 0. Particularly, the appearance of Formulations 2, 4, 6, and 8 always maintained at level 1

TABLE 13
Biological activity (%; compared to labled amount) under the condition of influence factors

| | | Formulation | | | | | | | | | |
|---|---|---|---|---|---|---|---|---|---|---|---|
| Condition | Time | 1 | 2 | 3 | 4 | 5 | 6 | 7 | 8 | 9 | 10 |
| | Day 0 | 101.0 | 99.7 | 99.8 | 96.5 | 99.6 | 99.8 | 99.8 | 99.5 | 99.7 | 99.5 |
| High | Day 7 | 93.6 | 98.7 | 97.9 | 93.2 | 100.2 | 98.9 | 98.2 | 99.3 | 98.6 | 96.1 |
| temperature | Day 14 | 85.9 | 98.2 | 94.6 | 90.8 | 98.6 | 98.6 | 98.6 | 98.7 | 98.9 | 92.8 |
| | Decline rate | 14.95 | 1.50 | 5.21 | 5.91 | 1.00 | 1.20 | 1.20 | 0.80 | 0.80 | 6.73 |
| | in 14 days | | | | | | | | | | |
| High | Day 7 | 99.8 | 99.6 | 100.2 | 96.7 | 99.7 | 99.8 | 99.9 | 99.6 | 99.4 | 99.7 |
| humidity | 14 day | 100.3 | 99.8 | 99.6 | 95.8 | 99.3 | 99.5 | 99.6 | 99.7 | 99.5 | 99.8 |
| | Decline rate | 0.69 | −0.10 | 0.20 | 0.73 | 0.30 | 0.30 | 0.20 | −0.20 | 0.20 | −0.30 |
| | in 14 days | | | | | | | | | | |
| Light | Day 7 | 96.5 | 98.5 | 99.1 | 95.3 | 99.4 | 98.9 | 99.3 | 98.8 | 98.0 | 94.5 |
| | Day 14 | 91.4 | 99.1 | 96.4 | 91.2 | 99.2 | 98.2 | 99.1 | 99.2 | 98.2 | 90.1 |
| | Decline rate | 9.50 | 0.60 | 3.41 | 5.49 | 0.40 | 1.60 | 0.70 | 0.30 | 1.50 | 9.45 |
| | in 14 days | | | | | | | | | | |

As shown in Table 13, the biological activity of Formulations 3, 4, and 10 decreased slightly after 14 days in high temperature or light condition, with a reduction rate between 3.41% and 9.45%, but still better than the control (Formulation 1). The biological activity of Formulations 2 and 5-9 basically remained stable under all tested conditions, with decline rate of less than 1.60% after 14 days in high temperature or light condition. Compared with FX activator formulations of the present invention (Formulations 2-10), biological activity of FX activator in the control sample (Formulation 1) reduced significantly. These results showed that under conditions of high temperature and light, human serum albumin (HSA) was insufficient for maintaining the stability or protecting of the active components (FX activator). In the contrary, the technical scheme of the present invention was able to provide more effective stability and protection.

The above results indicate that the technical scheme of the present invention can significantly and effectively stabilize and protect the activity of coagulation factor X activator under conditions of high temperature, high humidity, and light.

### III) Accelerated and Lone-Term Stability Tests

Stability tests were conducted on all formulations to investigate the changes of biological activity and insoluble particles.

### 1) Biological Activity Test

Formulations 1-10 were placed under conditions of acceleration (25° C., RH 65±5%) and long-term storage (2-8° C.) for stability investigation, to detect the change of biological activity. The results are shown in Table 14.

TABLE 14
Biological activity (%; compared to labled amount) under accelerated and
long-term storage conditions

| | | Formulation | | | | | | | | | |
|---|---|---|---|---|---|---|---|---|---|---|---|
| Condition | Time | 1 | 2 | 3 | 4 | 5 | 6 | 7 | 8 | 9 | 10 |
| | 0 month | 101.0 | 99.7 | 99.8 | 96.5 | 99.6 | 99.8 | 99.8 | 99.5 | 99.7 | 99.5 |
| Acceleration | 1 month | 96.7 | 99.1 | 99.0 | 95.3 | 101.3 | 99.1 | 99.0 | 100.7 | 98.5 | 98.9 |
| | 2 months | 96.5 | 98.9 | 97.5 | 93.2 | 99.2 | 98.3 | 98.6 | 99.2 | 98.9 | 98.2 |
| | 3 months | 93.2 | 98.2 | 96.1 | 93.3 | 99.8 | 98.4 | 98.7 | 98.8 | 99.0 | 98.5 |
| | 6 months | 88.7 | 98.5 | 94.3 | 89.1 | 98.3 | 98.2 | 98.2 | 98.9 | 98.3 | 97.3 |
| | Decline rate | 12.18 | 1.20 | 5.51 | 7.67 | 1.31 | 1.60 | 1.60 | 0.60 | 1.40 | 2.21 |
| Long-term | 3 months | 98.3 | 99.4 | 99.3 | 95.9 | 99.3 | 99.5 | 99.9 | 99.6 | 99.5 | 98.6 |
| | 6 months | 96.2 | 99.2 | 97.5 | 94.2 | 98.8 | 98.9 | 99.3 | 99.4 | 99.1 | 98.0 |
| | Decline rate | 4.75 | 0.50 | 2.30 | 2.38 | 0.80 | 0.09 | 0.50 | 0.10 | 0.60 | 1.51 |

The biological activity test results showed that after 6-month acceleration or long-term storage condition, the stability of lyophilized formulations prepared according to the technical scheme of the present invention (Formulations 2-10) was significantly better than that of control (Formulation 1); particularly, the activity reduction rate of Formulations 2 and 5-9 was less than 1.60% or 0.80% after 6-month accelerated or long-term storage, respectively. The above results indicated that the technical scheme of the present invention could make FX activator more stable under accelerated and long-term storage conditions.

### 2) Insoluble Particle Detection

The diameter of human capillaries is 7-12 μm. Insoluble particles may cause vascular embolism, induced phlebitis, granuloma, pulmonary hypertension, and pyrogen reaction during intravenous administration. Hence, for drugs to be administered intravenously, insoluble particles should be avoided or the amount thereof should be reduced. Insoluble particles should be detected during the preparation of such drugs.

Insoluble particles mainly arise from drug production process, compatibility process, and administration process. In addition, insoluble particles may arise during stirring, freeze-drying, and storage process of protein drugs.

The present invention adopted FlowCam® 8000 subvisible particle imaging analysis system, to capture particles dynamically flowing through the flow cell via the electronic imaging system, then analyzes size, shape, refraction, and other parameters of the insoluble particles through software, so as to distinguish all images, such as protein particles, silicone oil, bubbles, fibers, etc. The detectable range of particle size was 2-100 μm.

All injection powder formulations were dissolved with 2 ml saline after 0 month and 6 months of acceleration, then insoluble particles were examined based on 0903 insoluble particle inspection method in “Chinese Pharmacopoeia,” 2015 Edition, IV General Principles. Results are shown in Table 15 and Table 16 (background particles were deducted from both data).

TABLE 15
Insoluble particles in 0 month

| Classification of | Formulation | | | | | | | | | |
|---|---|---|---|---|---|---|---|---|---|---|
| insoluble particles | 1 | 2 | 3 | 4 | 5 | | 7 | 8 | 9 | 10 |
| 2-10 μm | 1230 | 165 | 612 | 367 | 153 | 161 | 213 | 229 | 216 | 138 |
| 10 μm+ | 25 | 4 | 13 | 9 | 5 | 7 | 6 | 6 | 7 | 6 |
| 25 μm+ | 5 | 0 | 4 | 0 | 0 | 0 | 0 | 0 | 0 | 0 |
| 2-10 μm (silicone oil) | 53 | 33 | 11 | 16 | 18 | 9 | 11 | 22 | 18 | 31 |
| 10 μm+ (silicone oil) | 26 | 12 | 13 | 7 | 7 | 6 | 3 | 0 | 9 | 14 |
| 25 μm+ (silicone oil) | 0 | 0 | 0 | 0 | 0 | 0 | 0 | 0 | 0 | 0 |
| Fiber | 0 | 0 | 0 | 0 | 0 | 0 | 0 | 0 | 0 | 0 |
| Total | 1339 | 214 | 653 | 399 | 183 | 183 | 233 | 257 | 250 | 189 |
| Percentage of | 96% | 93% | 95% | 96% | 93% | 93% | 96% | 98% | 94% | 89% |
| 2-10 μm particles | | | | | | | | | | |

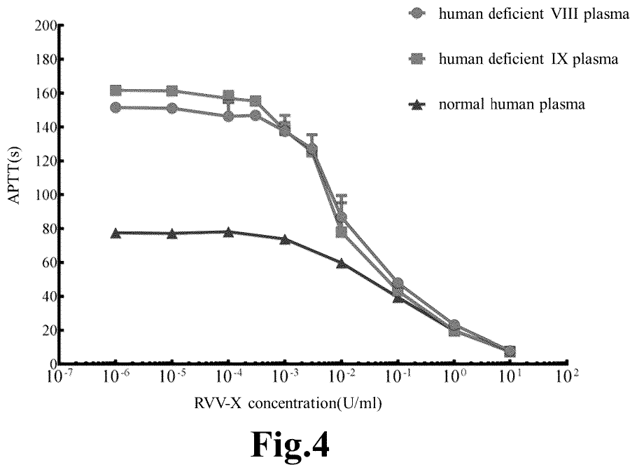

As shown in Table 15, the number of insoluble particles in each lyophilized formulation sample all met the requirements of Chinese Pharmacopoeia, but the number of insoluble particles of various sizes in the control sample (Formulation 1) was significantly higher than that in other lyophilized formulation samples. The number of insoluble particles in lyophilized formulations of the present invention was all at low level; particularly, the total number of insoluble particles was even lower than 260 in Formulations 2 and 5-10. These results demonstrate that the formulation scheme of the present invention could significantly reduce insoluble particles. Also see FIG. 5.

TABLE 16
Insoluble particles after 6 months of acceleration

| Classification of | Formulation | | | | | | | | | |
|---|---|---|---|---|---|---|---|---|---|---|
| insoluble particles | 1 | 2 | 3 | 4 | 5 | 6 | 7 | 8 | 9 | 10 |
| 2-10 μm | 11339 | 1815 | 5015 | 3052 | 1282 | 1346 | 1888 | 1795 | 1744 | 1213 |
| 10 μm+ | 193 | 34 | 53 | 68 | 34 | 44 | 33 | 24 | 38 | 26 |
| 25 μm+ | 35 | 0 | 11 | 0 | 0 | 0 | 0 | 0 | 0 | 0 |
| 2-10 μm (silicone oil) | 67 | 31 | 17 | 25 | 25 | 16 | 23 | 21 | 19 | 33 |
| 10 μm+ (silicone oil) | 20 | 10 | 10 | 5 | 5 | 5 | 6 | 0 | 9 | 14 |
| 25 μm+ (silicone oil) | 0 | 0 | 0 | 0 | 0 | 0 | 0 | 0 | 0 | 0 |
| Fiber | 0 | 0 | 0 | 0 | 0 | 0 | 0 | 0 | 0 | 0 |
| Total | 11654 | 1890 | 5106 | 3150 | 1346 | 1411 | 1950 | 1840 | 1810 | 1286 |
| Percentage of | 98% | 98% | 99% | 98% | 97% | 97% | 98% | 99% | 97% | 97% |
| 2-10 μm particles | | | | | | | | | | |

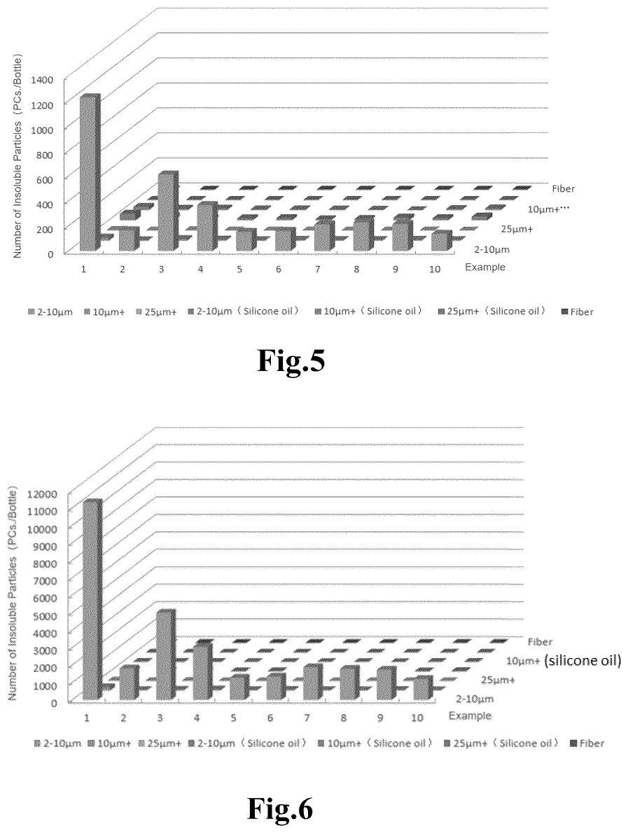

As shown in Table 16, insoluble particles in the control sample (Formulation 1) increased significantly after 6 months of acceleration storage, the majority were 2-10 μm in size. See FIG. 6. This was likely due to partial aggregation of human serum albumin in the formulation during storage, thus leading to increased insoluble particles content. Although the total amount of insoluble particles in the control sample was still within acceptable range, it was significantly higher than those in other formulation samples of the present invention. The total amount of insoluble particles was all at low level for formulation samples of the present invention; particularly, Formulations 2 and 5-10 had less than 2000 total insoluble particles. These results once again demonstrate that formulation scheme of the present invention could effectively improve the content of insoluble particles compared to the control formulation, so as to reduce the risk caused by insoluble particles and improve safety of clinical use.

To summarize, by using disaccharide (i.e., sucrose and/or trehalose) as stabilizer in combination with amino acid buffer (i.e., arginine and/or histidine), the obtained formulations not only significantly improved the stability of FX activator, but also avoided the potential risk caused by virus or other unknown components from using albumin as stabilizer. Especially, when the activity concentration of FX activator was 5 U/ml-50 U/ml, the mass volume concentration of stabilizer was 30 mg/ml-50 mg/ml, the mass volume concentration of buffer was 3 mg/ml-5 mg/ml, the mass volume concentration of surfactant was 0.1 mg/ml-0.3 mg/ml, the mass volume concentration of excipient was 30 mg/ml-60 mg/ml, and the pH of the formulation was 6.8-7.0, the appearance of the composition was exquisite and full, and the moisture content, reconstitution time, pH value, and FX activator biological activity all met product standard, and the stability of the composition remained great under high temperature, high humidity, light, acceleration, and long-term conditions.

### Example 7: Formulation Optimization

To further optimize FX activator formulations, a set of formulations were prepared according to Table 17 following method described in Example 5, to prepare semi-finished solution with concentration of 10 U/ml, 0.5 ml/vial (i.e., 5 U/vial). The formulation solution was then lyophilized into sterile powder for injection as described previously.

TABLE 17
Optimized FX activator formulation

| | | Formulation # | | | | | | | | | | | |
|---|---|---|---|---|---|---|---|---|---|---|---|---|---|
| Ingredient | Function | 6 | 11 | 12 | 13 | 14 | 15 | 16 | 17 | 18 | 19 | 20 | 21 |
| FX activator | Active ingredient | 10 | 10 | 10 | 10 | 10 | 10 | 10 | 10 | 10 | 10 | 5 | 50 |
| | (U/ml) | | | | | | | | | | | | |
| Sucrose | Stabilizer (mg/ml) | 50 | 30 | 30 | | 25 | 50 | 30 | 30 | 30 | 30 | 30 | 30 |
| Trehalose | | | | | 30 | | | | | | | | |
| Histidine | Buffering agent | 4 | 3 | | 3 | 3 | 3 | 2.5 | 5 | 3 | 3 | 3 | 3 |
| Arginine | (mg/ml) | | | 3 | | | | | | | | | |
| Polysorbate 20 | Surfactant (mg/ml) | 0.3 | 0.2 | 0.2 | 0.2 | 0.2 | 0.2 | 0.2 | 0.2 | 0.2 | 0.2 | 0.1 | 0.3 |
| Mannitol | Tonicity agent (mg/ml) | 40 | 40 | 40 | 40 | 40 | 40 | 40 | 40 | 30 | 60 | 40 | 40 |
| pH | | 6.80 | 6.85 | 6.85 | 6.85 | 6.8 | 6.85 | 6.9 | 6.85 | 6.85 | 6.9 | 7 | 6.7 |

After 0 month and 6 months of acceleration condition, lyophilized formulations were reconstituted with 2 ml normal saline. Insoluble particles were examined based on 0903 insoluble particle inspection method in “Chinese Pharmacopoeia,” 2015 Edition, IV General Principles. Results are shown in Tables 18-19.

TABLE 18
Insoluble particles in each formualation at 0 month

| Classification | | | | | | | | | | | | |
|---|---|---|---|---|---|---|---|---|---|---|---|---|
| of insoluble | Formulation # | | | | | | | | | | | |
| particles | 6 | 11 | 12 | 13 | 14 | 15 | 16 | 17 | 18 | 19 | 20 | 21 |
| 2-10 μm | 160 | 100 | 179 | 186 | 173 | 167 | 166 | 175 | 162 | 158 | 154 | 165 |
| 10 μm+ | 7 | 3 | 11 | 10 | 8 | 9 | 8 | 6 | 9 | 10 | 8 | 7 |
| 25 μm+ | 0 | 0 | 0 | 0 | 0 | 0 | 0 | 0 | 0 | 0 | 0 | 0 |
| 2-10 μm | 8 | 4 | 9 | 10 | 9 | 9 | 9 | 7 | 6 | 10 | 9 | 8 |
| (silicone oil) | | | | | | | | | | | | |
| 10 μm+ | 6 | 3 | 7 | 6 | 8 | 6 | 8 | 8 | 7 | 6 | 8 | 5 |
| (silicone oil) | | | | | | | | | | | | |
| 25 μm+ | 0 | 0 | 0 | 0 | 0 | 0 | 0 | 0 | 0 | 0 | 0 | 0 |
| (silicone oil) | | | | | | | | | | | | |
| Fiber | 0 | 0 | 0 | 0 | 0 | 0 | 0 | 0 | 0 | 0 | 0 | 0 |
| Total | 181 | 110 | 206 | 212 | 198 | 191 | 191 | 196 | 184 | 184 | 179 | 185 |

TABLE 19
Insoluble particles in each formulation after 6 months of acceleration

| Classification of | Formulation | | | | | | | | | | | |
|---|---|---|---|---|---|---|---|---|---|---|---|---|
| insoluble particles | 6 | 11 | 12 | 13 | 14 | 15 | 16 | 17 | 18 | 19 | 20 | 21 |
| 2-10 μm | 1340 | 889 | 1400 | 1398 | 1359 | 1364 | 1356 | 1369 | 1338 | 1346 | 1348 | 1336 |
| 10 μm+ | 45 | 30 | 55 | 51 | 48 | 45 | 47 | 45 | 43 | 47 | 45 | 46 |
| 25 μm+ | 0 | 0 | 0 | 0 | 0 | 0 | 0 | 0 | 0 | 0 | 0 | 0 |
| 2-10 μm (silicone oil) | 16 | 8 | 20 | 21 | 15 | 18 | 17 | 16 | 13 | 14 | 16 | 17 |
| 10 μm+ (silicone oil) | 5 | 2 | 6 | 7 | 5 | 6 | 7 | 6 | 5 | 6 | 7 | 7 |
| 25 μm+ (silicone oil) | 0 | 0 | 0 | 0 | 0 | 0 | 0 | 0 | 0 | 0 | 0 | 0 |
| Fiber | 0 | 0 | 0 | 0 | 0 | 0 | 0 | 0 | 0 | 0 | 0 | 0 |
| Total | 1406 | 929 | 1481 | 1477 | 1427 | 1433 | 1427 | 1436 | 1399 | 1413 | 1416 | 1406 |

As shown in Table 18, the number of insoluble particles in each lyophilized formulation sample all met the requirements of Chinese Pharmacopoeia. Of which, Formulation 11 had the lowest number of insoluble particles and the best stability. Table 19 illustrated that after 6 months of acceleration condition, the total amount of insoluble particles in each lyophilized formulation was all at a low level; especially in Formulation 11, the total amount of insoluble particles was even less than 1000.

### Example 8: Performance Test of FX Activator Semi-Finished Solution

Three batches (batch #: 20160306, 20160307, and 20160308) of semi-finished FX activator solution (0.5 mL/vial; not lyophilized) were prepared according to Formulation 11, followed by long-term stability study (below −70° C., 24 months), accelerated stability study (2-8° C., 6 months), and freeze-thaw stability study (freeze-thaw for 5 times, −70° C./2~8° C.). The test indexes included protein content, biological activity (relative to labeled amount), pH, and purity (by SEC-HPLC).

### I. Lone-Term Stability Study

Three batches of semi-finished FX activator solution were stored at −70° C. for 24 months, sampled and tested in the 3^{rd}, 6^{th}, 9^{th}, 12^{th}, 18^{th}, and 24^{th} month.

TABLE 20
Results of long-term stability of semi-finished solution

| | | | | | | | | | Change in |
|---|---|---|---|---|---|---|---|---|---|
| | | | | | | | | | 24-month |
| | | | | | | | | | relative to |
| Index | Batch #. | 0-month | 3-month | 6-month | 9-month | 12-month | 18-month | 24-month | 0-month |
| Protein | 20160306 | 0.47 | 0.52 | 0.48 | 0.51 | 0.52 | 0.50 | 0.48 | 0.01 |
| content | 20160307 | 0.49 | 0.51 | 0.50 | 0.53 | 0.53 | 0.53 | 0.51 | 0.02 |
| (mg/ml) | 20160308 | 0.50 | 0.52 | 0.52 | 0.54 | 0.54 | 0.54 | 0.53 | 0.03 |
| Biological | 20160306 | 99.9% | 100.1% | 99.7% | 99.3% | 99.8% | 99.1% | 99.2% | −0.7% |
| activity | 20160307 | 100.2% | 100.3% | 99.9% | 99.9% | 100.0% | 99.9% | 99.8% | −0.4% |
| (relative to | 20160308 | 100.5% | 100.2% | 100.0% | 99.9% | 99.5% | 99.8% | 99.9% | −0.6% |
| labeled | | | | | | | | | |
| amount, %) | | | | | | | | | |
| pH | 20160306 | 6.82 | 6.73 | 6.75 | 6.81 | 6.90 | 6.85 | 6.90 | 0.08 |
| | 20160307 | 6.82 | 6.82 | 6.73 | 6.82 | 6.91 | 6.84 | 6.91 | 0.09 |
| | 20160308 | 6.71 | 6.71 | 6.72 | 6.77 | 6.83 | 6.88 | 6.90 | 0.19 |
| Purity | 20160306 | 98.8% | 99.4% | 99.5% | 98.7% | 99.6% | 99,3% | 98.8% | 0 |
| (SEC- | 20160307 | 99.0% | 99.4% | 99.5% | 98.9% | 99.2% | 98.7% | 98.4% | −0.6% |
| HPLC) | 20160308 | 99.0% | 99.6% | 99.3% | 98.9% | 99.3% | 99.2% | 99.2% | 0.2% |

As shown in Table 20, the protein content changed within 0.01 mg/ml~0.03 mg/ml, the biological activity changed within −0.4%~−0.7/o, the pH value changed within 0.08~0.19, and the purity detected by SEC-HPLC changed within −0.6% ~0.2% relative to 0 month after the three batches of semi-finished solution were stored at −70° C. for 24 months. These results illustrated that there was very little or merely no change for each test index in the semi-finished FX activator solution (Formulation 11), which was all within acceptable standard range.

In conclusion, there was barely any difference in each test index of the semi-finished FX activator solution, the consistency among batches was high, and the stability of the formulation was great when kept at −70° C. for 24 months.

### II. Accelerated Stability Test

The above three batches of semi-finished FX activator solution were placed at 2-8° C., sampled and tested in the 1^{st}, 2^{nd}, 3^{rd}, and 6^{th} month respectively.

TABLE 21
Results of accelerated stability of semi-finished solution

| | | | | | | | Change in 6- |
|---|---|---|---|---|---|---|---|
| | | | | | | | month relative |
| Index | Batch #. | 0-month | 1-month | 2-month | 3-month | 6-month | to 0-month |
| Protein content | 20160306 | 0.47 | 0.47 | 0.47 | 0.52 | 0.47 | 0 |
| (mg/ml) | 20160307 | 0.49 | 0.49 | 0.49 | 0.58 | 0.49 | 0 |
| | 20160308 | 0.50 | 0.50 | 0.50 | 0.59 | 0.50 | 0 |
| Biological | 20160306 | 99.9% | 100.0% | 99.9% | 99.7% | 99.8% | −0.1% |
| activity (relative | 20160307 | 100.2% | 99.9% | 100.0% | 99.9% | 99.7% | −0.5% |
| to labeled | 20160308 | 100.8% | 100.3% | 100.4% | 99.9% | 100.2% | −0.6% |
| amount, %) | | | | | | | |
| pH | 20160306 | 6.82 | 6.9 | 6.8 | 6.8 | 6.72 | −0.10 |
| | 20160307 | 6.82 | 7.0 | 6.8 | 6.8 | 6.82 | 0 |
| | 20160308 | 6.71 | 6.8 | 6.8 | 6.7 | 6.80 | 0.09 |
| Purity | 20160306 | 98.8% | 99.4% | 99.1% | 99.8% | 99.6% | 0.8% |
| (SEC-HPLC) | 20160307 | 99.0% | 99.2% | 99.1% | 99.6% | 99.7% | 0.7% |
| | 20160308 | 99.0% | 99.2% | 99.0% | 99.6% | 99.8% | 0.8% |

As shown in Table 21, the protein content was unchanged, the biological activity changed within −0.1%~0.6%, the pH changed within −0.1~0.09, and purity detected by SEC-HPLC changed within 0.7%~0.8% relative to 0 month after three batches of semi-finished solutions were stored at 2~8° C. for 6 months. These results illustrated that there was very little or merely no change for each test index in the semi-finished FX activator solution (Formulation 11), which was all within acceptable standard range.

In conclusion, there was little or nearly no change in each test index of the semi-finished FX activator solution, the stability of the formulation was great when kept at 2~8° C. for 6 months.

### III. The Freeze-Thaw Stability Test

The above three batches of semi-finished FX activator solution were repeatedly frozen and thawed (frozen at −70′C for 24 hours, and thawed at 2~8° C.) once, twice, three times, four times, and five times, then sampled and tested respectively. After 5 times of freezing and thawing, the protein content of the three batches of semi-finished FX activator solution had almost no change, the biological activity changed within −0.5%~0.7%, the pH changed within 0.02~0.11, and the purity detected by SEC-HPLC changed within 0.5%~0.8% relative to non-freeze/thawed semi-finished FX activator solution. These results illustrated that there was very little or merely no change for each test index in the semi-finished FX activator solution (Formulation 11), which was all within acceptable standard range.

To summarize, the semi-finished FX activator solution (Formulation 11 non-lyophilized) could remain stable after repeatedly freezing and thawing for 5 times (freezing at −70° C. for 24 h and thawing at 2~8° C.).

### Example 9: Stability Studies of Finished Products of FX Activator Formulations

Three batches (batch #: 201606008, 201606009, 201606010) of semi-finished FX activator solution for injection (0.5 ml/vial) were prepared according to Formulation 11. The finished products were obtained after freeze-drying. Long-term stability study (2~8° C., 48 months) and accelerated stability study (25° C.±2° C., 6 months) were conducted respectively. Appearance, insoluble particles, pH value, osmotic pressure, moisture content, and biological activity (relative to labeled amount, %) and other indexes were tested.

### I. Lone-Term Stability Study of Finished Products of FX Activator Formulation

The above three batches of lyophilized FX activator formulation for injection were stored at 2~8° C. for 30 months, and sampled and tested in the 9^{th}, 18^{th}, and 30^{th} month, respectively.

TABLE 22
Results of long-term stability test of three batches of finished products

| | | | | | | Change in 30- |
|---|---|---|---|---|---|---|
| | | | | | | month relative |
| Index | Batch # | 0-month | 9-month | 18-month | 30-month | to 0-month |
| pH | 201606008 | 6.80 | 6.92 | 6.82 | 7.00 | 0.20 |
| | 201606009 | 6.80 | 6.91 | 6.80 | 6.90 | 0.10 |
| | 201606010 | 6.71 | 6.91 | 6.85 | 6.90 | 0.19 |
| Moisture | 201606008 | 2.4% | 1.6% | 1.6% | 1.6% | −0.8% |
| content | 201606009 | 2.3% | 1.8% | 1.6% | 1.6% | −0.7% |
| | 201606010 | 2.4% | 1.8% | 1.9% | 2.0% | −0.4% |
| Biological | 201606008 | 101.0% | 100.9% | 99.2% | 99.2% | −1.8% |
| activity (relative | 201606009 | 99.9% | 99.7% | 98.9% | 99.4% | −0.5% |
| to labelled | 201606010 | 100.2 | 99.9% | 100.1% | 97.9% | −2.3% |
| amount, %) | | | | | | |

The appearance and insoluble particles all met the standard after 30 months of storage at 2~8° C. As can be seen from Table 22, The pH value changed within 0.10~0.20, the moisture content changed within −0.4%~0.8%, and the biological activity changed within −0.5%~−2.3% relative to 0 month after storing at 2~8° C. for 30 months. These results illustrated that the change of each test index of the finished product of FX activator formulation was little and within the acceptable standard range after being stored at 2~8° C. for 30 months.

In conclusion, the finished product of FX activator formulation (lyophilized) had great stability after being stored at 2~8° C. for 30 months.

### II. Accelerated Stability Test of Finished Products of FX Activator Formulation

The above three batches of lyophilized FX activator formulations (finished product) were stored at 25° C.±2° C. for 6 months, then sampled and tested in the 0^{th}, 1^{st}, 2^{nd}, 3^{rd} and 6^{th} month respectively.

TABLE 23
Results of accelerated stability test of three batches of finished products

| | | | | | | | Change in 6- |
|---|---|---|---|---|---|---|---|
| | | | | | | | month relative |
| Index | Batch # | 0-month | 1-month | 2-month | 3-month | 6-month | to 0-month |
| Moisture | 201606008 | 2.4% | 2.2% | 1.9% | 1.9% | 1.8% | −0.6% |
| content | 201606009 | 2.3% | 2.2% | 1.8% | 1.8% | 1.9% | −0.4% |
| | 201606010 | 2.4% | 2.6% | 2.2% | 2.1% | 2.1% | −0.3% |
| pH | 201606008 | 6.80 | 6.81 | 6.68 | 6.81 | 6.80 | 0 |
| | 201606009 | 6.80 | 6.80 | 6.78 | 6.80 | 6.81 | 0.01 |
| | 201606010 | 6.71 | 6.72 | 6.71 | 6.80 | 6.81 | 0.10 |
| Biological | 201606008 | 101.0% | 99.1% | 100.8% | 99.9% | 100.1% | −0.9% |
| activity | 201606009 | 99.9% | 99.8% | 100.0% | 99.8% | 98.9% | −1.0% |
| (relative to | 201606010 | 100.2 | 101.0% | 99.8% | 99.7% | 99.9% | −0.3% |
| labelled | | | | | | | |
| amount, %) | | | | | | | |

The appearance and insoluble particles of three batches of finished products (lyophilized) all met the standard after being accelerated for 6 months at 25° C.±2° C. As shown in Table 23, the moisture content changed within −0.3%~−0.6%, the pH value changed within 0~0.1, and the biological activity changed within −0.3%~1.0% relative to 0 month after the finished product was stored for 6 months under acceleration (25° C.±2′C). These results illustrated that the change of each test index of the finished product of FX activator formulation was little or nerely none, and within the acceptable standard range after being stored at 25° C.±2° C. for 6 months.

In conclusion, the finished product of FX activator formulation (lyophilized) had great stability after being stored at room temperature of 25° C.±2° C. for 6 months.

### Example 10: Solvent Compatibility Study of FX Activator Lyophilized Formulations

Product of the invention (FX activator formulation) can be prepared as sterile powder for injection after freeze-drying. Product of the dosage form needs to be dissolved with an appropriate amount of solvent before use, then administered intravenously. To study solvent compatibility and its impact on protein stability, sterilized water for injection, 0.9% sodium chloride injection, and 5% glucose injection were selected as solvent according to clinically commonly used compatible medication methods. Formulation stability was examined at the starting and ending time points (shelf life or reporting time) to study solvent compatibility, to provide basis for clinical medication.

Lyophilized Formulation 6 and Formulation 11 were selected as exemplary formulations, reconstituted with 2m sterilized water for injection, 0.9% sodium chloride injection, or 5% glucose solution injection, respectively, and gently shaken to obtain uniform solution. Reconstituted Formulation 6 and Formulation 11 solution were placed at room temperature (25° C.) for 0, 2, 4, 6, and 8 hrs, the appearance of the solution was recorded, and pH, osmotic pressure, activity, insoluble particles, endotoxin, biological activity (relative to labeled amount), and sterility were recorded.

TABLE 24
Results of compatibility stability test for Formulation 6

| | | Time (h) | | | | |
|---|---|---|---|---|---|---|
| Solvent | Index | 0 | 2 | 4 | 6 | 8 |
| 2 ml sterilized | Appearance | Colorless | Colorless | Colorless | Colorless | Colorless |
| | | clear liquid | clear liquid | clear liquid | clear liquid | clear liquid |
| water for | pH | 6.78 | 6.81 | 6.80 | 6.79 | 6.82 |
| injection | Osmotic pressure | 102 | 100 | 101 | 102 | 103 |
| | Activity (%) | 97% | 102% | 96% | 80% | 76% |
| 2 ml 0.9% | Appearance | Colorless | Colorless | Colorless | Colorless | Colorless |
| | | clear liquid | clear liquid | clear liquid | clear liquid | clear liquid |
| sodium | pH | 6.82 | 6.81 | 6.82 | 6.80 | 6.84 |
| chloride | Osmotic pressure | 386 | 389 | 390 | 390 | 388 |
| injection | Activity (%) | 105% | 103% | 106% | 100% | 101% |
| 2 ml 5% | Appearance | Colorless | Colorless | Colorless | Colorless | Colorless |
| glucose | | clear liquid | clear liquid | clear liquid | clear liquid | clear liquid |
| injection | pH | 6.78 | 6.81 | 6.83 | 6.81 | 6.84 |
| | Osmotic pressure | 363 | 365 | 369 | 368 | 370 |
| | Activity (%) | 95% | 102% | 98% | 87% | 81% |

TABLE 25
Results of compatibility stability test for Formulation 11

| | | Time (h) | | | | |
|---|---|---|---|---|---|---|
| Solvent | Index | 0 | 2 | 4 | 6 | 8 |
| 2 ml sterile | Appearance | Colorless | Colorless | Colorless | Colorless | Colorless |
| | | clear liquid | clear liquid | clear liquid | clear liquid | clear liquid |
| water for | pH | 6.82 | 6.84 | 6.83 | 6.85 | 6.84 |
| injection | Osmotic pressure | 101 | 102 | 100 | 101 | 102 |
| | Activity (%) | 101.0% | 102.0% | 98.9% | 88.3% | 82.2% |
| 2 ml 0.9% | Appearance | Colorless | Colorless | Colorless | Colorless | Colorless |
| | | clear liquid | clear liquid | clear liquid | clear liquid | clear liquid |
| sodium chloride | pH | 6.85 | 6.87 | 6.86 | 6.84 | 6.85 |
| injection | Osmotic pressure | 379 | 386 | 381 | 385 | 376 |
| | Activity (%) | 100.0% | 103.1% | 102.1% | 100.8% | 101.0% |
| 2 ml 5% | Appearance | Colorless | Colorless | Colorless | Colorless | Colorless |
| glucose | | clear liquid | clear liquid | clear liquid | clear liquid | clear liquid |
| injection | pH | 6.84 | 6.82 | 6.85 | 6.86 | 6.83 |
| | Osmotic pressure | 365 | 361 | 370 | 368 | 364 |
| | Activity (%) | 102.0% | 100.5% | 99.9% | 95.8% | 90.8% |

As shown in Table 24 and Table 25, after reconstitution with three different solvents and placed at room temperature for 8 hours, the appearance of reconstituted Formulation 6 and Formulation 11 were both colorless and clear liquid, and pH did not change significantly. Formulation 11 after reconstitution with 2 mL 0.9% sodium chloride injection was used for administration in above Examples 1, 2, 4.

Normal osmotic pressure of human plasma is 280-320 mOsm/kg. The osmotic pressure of reconstituted Formulation 6 by 2 ml water for injection was about 85 mOsm/kg, the osmotic pressure of reconstituted Formulation 11 by 2 ml water for injection was about 100 mOsm/kg, which were both lower than normal osmotic pressure of human plasma. The osmotic pressure of reconstituted Formulation 6 and Formulation 11 by 2 ml 0.9% sodium chloride injection or 5% glucose injection was both about 360-400 mOsm/kg, which was higher than normal osmotic pressure of human plasma. Although none of the reconstituted formulations was isotonic by the three solvents, administration of FX activator formulation of the subject invention should not have adverse impact on plasma osmotic pressure, because the product is for intravenous administration with small volume, and human plasma has certain buffering capacity.

Regarding stability of FX activator activity, FX activator biological activity remained stable after reconstituting with 2 ml 0.9% sodium chloride injection and placing at room temperature for 8 hours for both Formulation 6 and Formulation 11. FX activator activity was basically stable after reconstituting Formulation 6 with 2 ml sterilized water for injection or 5% glucose injection and placing at room temperature for 4 hours. FX activator activity was basically stable after reconstituting Formulation 11 with 2 ml sterilized water for injection or 5% glucose injection and placing at room temperature for 6 hours.

The study on the stability of FX activator formulation before and after freeze-drying and solvent compatibility stability illustrated that the stability of Formulation 11 was great. Formulation 11 was prepared as 1.0 ml/vial with biological activities of 5 U/ml, 50 U/ml, 10 U/ml, or 100 U/ml (i.e. 5 U/vial, 50 U/vial, 10 U/vial, or 100 U/vial), respectively. The stability of the formulation of above specifications was investigated under various influencing factors. The results showed that under various conditions, the preparation of above specifications according to Formulation 11 (other components not changing, only changing RVV-X amount) could relatively maintain stable in key indexes such as moisture content, pH, osmotic pressure, and biological activity, indicating that Formulation 11 had great stabilizing effect on RVV-X preparations of different specifications (results not shown).

Although the invention has been described in detail with general description, specific implementation method, and examples above, based on the present invention, those skilled in the art can make modifications and improvements within the scope and not deviating from the main purpose of the invention, and these modifications or improvements also belong to the protection scope of the invention.

1-123. (canceled)

124. A method of treating a bleeding disorder in an individual, comprising administering to the individual an effective amount of an FX activator, wherein the FX activator is administered in a dose of from about 0.01 U/kg to about 0.48 U/kg.

125. The method of claim 124, wherein:
(i) the FX activator is administered in a dose of from about 0.08 U/kg to about 0.48 U/kg or about 0.1 U/kg to about 0.48 U/kg, or
(ii) the FX activator is administered in a dose of from about 0.01 U/kg to about 0.16 U/kg or about 0.1 U/kg to about 0.16 U/kg, or
(iii) the FX activator is administered in a dose of from about 0.08 U/kg to about 0.16 U/kg, or
(iv) the FX activator is administered in a dose of about 0.1 U/kg, 0.12 U/kg, 0.14 U/kg or 0.16 U/kg.

126. The method of claim 124, wherein:
(i) the FX activator is administered once, or
(ii) the FX activator is administered for a maximum of 6 doses, or
(iii) the FX activator is administered for 4 doses.

127. The method of claim 124, wherein:
(i) the FX activator is administered every 4 hours (q4h) to every 8 hours (q8h), or
(ii) the FX activator is administered q4h.

128. The method of claim 124, wherein:
(i) the FX activator is administered once in a dose of from about 0.01 U/kg to about 0.48 U/kg, or
(ii) the FX activator is administered in a dose of about 0.1 U/kg or 0.16 U/kg q4h for a maximum of 6 doses, or
(iii) the FX activator is administered in a dose of about 0.1 U/kg or 0.16 U/kg q4h for 4 doses.

129. The method of claim 124, wherein the FX activator is RVV-X, optionally,
wherein the RVV-X is isolated from Daboia russellii siamensis venom, optionally,
wherein the purity of the RVV-X is at least about 95%.

130. The method of claim 124, wherein the FX activator is contained in a pharmaceutical composition, wherein:
(i) the concentration of the FX activator in the pharmaceutical composition is from about 0.1 U/mL to about 200 U/mL, or
(ii) the concentration of the FX activator in the pharmaceutical composition is from about 1 U/mL to about 100 U/mL, or
(iii) the concentration of the FX activator in the pharmaceutical composition is from about 5 U/mL to about 100 U/mL, or
(iv) the concentration of the FX activator in the pharmaceutical composition is from about 5 U/mL to about 50 U/mL, or
(v) the concentration of the FX activator in the pharmaceutical composition is about 10 U/mL.

131. The method of claim 130, wherein the pharmaceutical composition further comprises one or more of a stabilizer, a buffering agent, a surfactant, and a tonicity agent.

132. The method of claim 131, wherein:
(i) the stabilizer comprises one or both of sucrose and trehalose, and/or
(ii) wherein the stabilizer is in an amount of from about 2 mg/ml to about 100 mg/ml, or
(iii) wherein the stabilizer is in an amount of from about 2 mg/ml to about 60 mg/ml, or
(iv) wherein the stabilizer is in an amount of from about 15 mg/ml to about 60 mg/ml, or
(v) wherein the stabilizer is in an amount of from about 30 mg/ml to about 50 mg/ml, or
(vi) wherein the stabilizer is in an amount of about 30 mg/ml.

133. The method of claim 131, wherein:
(i) the buffering agent comprises one or both of histidine and arginine, and/or
(ii) the buffering agent is in an amount of from about 0.1 mg/ml to about 50 mg/ml, or
(iii) the buffering agent is in an amount of from about 2 mg/ml to about 20 mg/ml, or
(iv) the buffering agent is in an amount of from about 2 mg/ml to about 15 mg/ml, or
(v) the buffering agent is in an amount of from about 3 mg/ml to about 5 mg/ml, or
(vi) the buffering agent is in an amount of about 3 mg/ml.

134. The method of claim 131, wherein:
(i) the surfactant comprises one or both of polysorbate and poloxamer, or,
(ii) the surfactant is selected from one or more of polysorbate 20, polysorbate 80, and poloxamer 188, and/or
(iii) wherein the surfactant is in an amount of from about 0.001% (w/v) to about 0.1% (w/v), or
(iv) wherein the surfactant is in an amount of from about 0.005% (w/v) to about 0.05% (w/v), or
(v) wherein the surfactant is in an amount of from about 0.01% (w/v) to about 0.05% (w/v), or
(vi) wherein the surfactant is in an amount of from about 0.01% (w/v) to about 0.03% (w/v), or
(vii) wherein the surfactant is in an amount of about 0.02% (w/v).

135. The method of claim 131, wherein:
(i) the tonicity agent is mannitol, and/or
(ii) wherein the tonicity agent is in an amount of from about 1 mg/ml to about 100 mg/ml, or
(iii) wherein the tonicity agent is in an amount of from about 10 mg/ml to about 60 mg/ml, or
(iv) wherein the tonicity agent is in an amount of from about 30 mg/ml to about 60 mg/ml, or
(v) wherein the tonicity agent is in an amount of about 40 mg/ml.

136. The method of claim 130, wherein:
(i) the pharmaceutical composition has a pH of from about 6.0 to about 8.0, or
(ii) wherein the pharmaceutical composition has a pH of from about 6.3 to about 7.3, or
(iii) wherein the pharmaceutical composition has a pH of from about 6.8 to about 7.0, or
(iv) wherein the pharmaceutical composition has a pH of about 6.85.

137. The method of claim 130, wherein the pharmaceutical composition further comprises an antioxidant and/or a calcium salt.

138. The method of claim 137, wherein: (i) the antioxidant is methionine, and/or
(ii) the antioxidant is in an amount of from about 0.01 mg/ml to about 1 mg/ml, or
(iii) the antioxidant is in an amount of from about 0.05 mg/ml to about 1 mg/ml.

139. The method of claim 137, wherein:
(i) the calcium salt is calcium chloride, and/or
(ii) the calcium salt is in an amount of from about 0.1 mg/ml to about 10 mg/ml.

140. The method of claim 130, wherein the pharmaceutical composition comprises:
i) RVV-X in an amount of about 10 U/mL;
ii) sucrose in an amount of about 30 mg/ml;
iii) histidine in an amount of about 3 mg/ml;
iv) polysorbate 20 in an amount of about 0.02% (w/v); and
v) mannitol in an amount of about 40 mg/ml;
wherein the pharmaceutical composition has a pH of about 6.85.

141. The method of claim 124, wherein the FX activator is administered intravenously, and/or, wherein the individual is a human.

142. The method of claim 124, wherein:
(i) the bleeding disorder is a congenital bleeding disorder or an acquired bleeding disorder, or,
(ii) the bleeding disorder is selected from the group consisting of a disorder due to a deficiency of a coagulation factor, a disorder due to the presence of acquired inhibitors to a coagulation factor, a hematologic disorder, a hemorrhagic disorder, Von Willebrands' disease, a disorder resulting from anticoagulant therapy with a vitamin-K antagonist, hereditary platelet disorders, vitamin K epoxide reductase Cl deficiency, gamma-carboxylase deficiency, bleeding associated with trauma, injury, or surgery, thrombosis, thrombocytopenia, stoke, coagulopathy, disseminated intravascular coagulation (DIC), Bernard Soulier syndrome, Glanzman thromblastemia, and storage pool deficiency, or
(iii) the bleeding disorder is hemophilia, optionally,
(iv) wherein the hemophilia is hemophilia A or hemophilia B, and/or
(v) wherein the hemophilia is hemophilia with inhibitors.

143. The method of claim 129, wherein the RVV-X comprises:
a) i) a heavy chain comprising the sequence of SEQ ID NO: 1, or a sequence with at least about 80% identity to SEQ ID NO: 1; ii) a light chain 1 comprising the sequence of SEQ ID NO: 2, or a sequence with at least about 80% identity to SEQ ID NO: 2; and iii) a light chain 2 comprising the sequence of SEQ ID NO: 3, or a sequence with at least about 80% identity to SEQ ID NO: 3;
b) i) a heavy chain comprising the sequence of SEQ ID NO: 1, or a sequence with at least about 80% identity to SEQ ID NO: 1; ii) a light chain 1 comprising the sequence of SEQ ID NO: 5, or a sequence with at least about 80% identity to SEQ ID NO: 5; and iii) a light chain 2 comprising the sequence of SEQ ID NO: 3, or a sequence with at least about 80% identity to SEQ ID NO: 3; or
c) a mixture of a) and b).


SEQUENCE LISTING (5 sequences)

SEQ ID NO: 1 - protein, 431 aa, synthetic construct
  REGION 1..431: note = RVV-X heavy chain or alpha chain
LVATSAQFNKAFIELIIIVDHSMAKKCNSTATNTKIYEIVNSANEIFNPLNIHVTLIGVEFWCDRDLINVTSSADETLDSFGEWRASDLMTRKSHDNALLFTDMRFDLNTLGITFLAGMCQAYRSVGIVQVQGNRNFKTAVIMAHELSHNLGMYHDGKNCICNDSSCVMSPVLSDQPSKLFSNCSIHDYQRYLTRYKPKCILYPPLRKDIVSPPVCGNEIWEEGEECDCGSPADCQNPCCDAATCKLKPGAECGNGLCCYQCKIKTAGTVCRRARNECDVPEHCTGQSAECPRDQLQQNGQPCQNNRGYCYNGDCPIMRNQCISLFGSRANVAKDSCFQENLKGSYYGYCRKENGRKIPCAPQDVKCGRLFCLNNSPGNKNPCNMHYSCMDQHKGMVDPGTKCEDGKVCNNKRQCVDVNTAYQSTTGFSQI

SEQ ID NO: 2 - protein, 123 aa, synthetic construct
  REGION 1..123: note = RVV-X light chain 1 or gamma chain
VLDCPSGWLSYEQHCYKGFNDLKNWTDAEKFCTEQKKGSHLVSLHSREEEEFVVNLISENLEYPATWIGLGNMWKDCRMEWSDRGNVKYKALAEESYCLIMITHEKEWKSMTCNFIAPVVCKF

SEQ ID NO: 3 - protein, 135 aa, synthetic construct
  REGION 1..135: note = RVV-X light chain 2 or beta chain
GLDCPPDSSLYRYFCYRVFKEHKTWEAAERFCMEHPNNGHLVSVESMEEAEFVAKLLSNITEKFITHFWIGLMIKDKEQECSSEWSDGSSVSYDNLDKREFRKCFVLEKESGYRMWFNRNCEERYLFVCKVPPEC

SEQ ID NO: 4 - protein, 4 aa, synthetic construct
  CARBOHYD 1: note = Suc
  BINDING 4: note = pNA
  BINDING 2: note = gammaPip
IGGR

SEQ ID NO: 5 - protein, 123 aa, synthetic construct
  REGION 1..123: note = RVV-X light chain 1 or gamma chain
VLDCPSGWLSYEQHCYKGFNDLKNWTDAEKFCTEQKKGSHLVSLHSREEEKFVVNLISENLEYPATWIGLGNMWKDCRMEWSDRGNVKYKALAEESYCLIMITHEKVWKSMTCNFIAPVVCKF